(19) United States
(12) Patent Application Publication
(10) Pub. No.: US 2024/0075006 A1
(43) Pub. Date: Mar. 7, 2024

(54) USE OF FERRIC CITRATE IN THE TREATMENT OF IRON-DEFICIENCY ANEMIA

(71) Applicant: Keryx Biopharmaceuticals, Inc., Cambridge, MA (US)

(72) Inventors: Enrique Poradosu, Brookline, MA (US); Ron Bentsur, Tenafly, NJ (US); Shay David Shemesh, Camas, WA (US)

(21) Appl. No.: 18/069,555

(22) Filed: Dec. 21, 2022

Related U.S. Application Data
(63) Continuation of application No. 17/743,196, filed on May 12, 2022, now abandoned.
Application No. 17/743,196 is a continuation of application No. 17/490,195, filed on Sep. 30, 2021, now abandoned.
Application No. 17/490,195 is a continuation of application No. 15/553,348, filed on Aug. 24, 2017, now abandoned.
(60) Provisional application No. 62/127,963, filed on Mar. 4, 2015.

(51) Int. Cl. A61K 31/295; A61K 9/00; A61K 9/20

(52) U.S. Cl. CPC A61K 31/295; A61K 9/0053; A61K 9/20; A61K 9/2059

(57) ABSTRACT

Described herein are methods fir treating patients with iron-deficiency anemia (IDA), comprising administering ferric citrate to such patients. In certain aspects, the patients treated for iron-deficiency anemia have a gastrointestinal disorder, such as inflammatory bowel disease, inflammatory bowel syndrome, Crohn's disease, microscopic colitis (such as collagenous or lymphocytic colitis), or chemically-induced colitis (e.g., NSAID (nonsteroidal anti-inflammatory drug)-induced colitis). In certain aspects, the patients treated for iron-deficiency anemia have blood loss associated with childbirth, menstruation or infection. In some aspects, the patients treated for iron-deficiency anemia have insufficient dietary intake of iron and/or insufficient absorption of iron.

### CROSS-REFERENCE TO RELATED APPLICATIONS

This application claims the benefit of U.S. provisional application No. 62/127,963, filed on Mar. 4, 2015, which is incorporated by reference herein in its entirety.

### 1. FIELD

Described herein are methods for treating patients with iron-deficiency anemia (IDA), comprising administering ferric citrate to such patients. In certain aspects, the patients treated for iron-deficiency anemia have a gastrointestinal disorder, such as inflammatory bowel disease, inflammatory bowel syndrome, Crohn's disease, microscopic colitis (such as collagenous or lymphocytic colitis), or chemically-induced colitis (e.g., NSAID (nonsteroidal anti-inflammatory drug)-induced colitis). In certain aspects, the patients treated for iron-deficiency anemia have blood loss associated with childbirth, menstruation or infection. In some aspects, the patients treated for iron-deficiency anemia have insufficient dietary intake of iron and/or insufficient absorption of iron.

### 2. BACKGROUND

About 2 billion people have anemia world-wide, and iron deficiency is the most prevalent cause of anemia, affecting millions of children, women, and men in both developed and less developed countries (Baltussen et al., Journal of Nutrition (2004) 134, 2678-2684; McLean et al., Public Health Nutr. (2009) 12, 444-454). Although the impact of iron-deficiency anemia (IDA) on human health is significant, it is either frequently overlooked or insufficiently treated (Miller et al., Cold Spring Harb. Perspect. Med. (2013) 3, a011866).

Most well-nourished, non-iron deficient people living in industrialized countries have approximately 4 to 5 grams of iron stored within their bodies in some manner (e.g., as circulating iron or stored iron or both). A decrease in this amount represents an iron deficiency, which is commonly seen in IDA patients. Symptoms of iron deficiency can occur in the patients before the condition has progressed to IDA, and can include, for example, fatigue, dizziness, pallor, hair loss, irritability, weakness, pica, brittle or grooved nails, Plummer-Vinson syndrome (painful atrophy of the mucous membrane covering the tongue, pharynx and esophagus), impaired immune function, pagophagia, and restless legs syndrome, among others.

IDA is typically characterized by pallor (pale color resulting from reduced oxyhemoglobin in the skin and mucous membranes), fatigue, lightheadedness, and weakness. However, signs of IDA can vary among patients. Because iron deficiency in IDA patients tends to develop slowly, adaptation to the disease can occur and it can go unrecognized for some time, even years. In some instances, patients with IDA can develop dyspnea (trouble breathing), pica (unusual obsessive food cravings), anxiety often resulting in obsessive-compulsive disorder (OCD)-type compulsions and obsessions, irritability or sadness, angina, constipation, sleepiness, tinnitus, mouth ulcers, palpitations, hair loss, fainting or feeling faint, depression, breathlessness on exertion, twitching muscles, pale yellow skin, tingling (numbness) or burning sensations, missed menstrual cycle(s), heavy menstrual period(s), slow social development, glossitis (inflammation or infection of the tongue), angular cheilitis (inflammatory lesions at the mouth's corners), koilonychia (spoon-shaped nails) or nails that are weak or brittle, poor appetite, pruritus (generalized itchiness), Plummer-Vinson syndrome (painful atrophy of the mucous membrane covering the tongue, pharynx and esophagus), insomnia, and restless legs syndrome, among others.

IDA can be caused by insufficient dietary intake of iron, insufficient absorption of iron, insufficient storage of iron, and/or iron loss from bleeding which can originate from a number of sources such as the gastrointestinal, uterine or urinary tract. Therefore it is commonly associated with conditions and disorders such as acute blood loss, chronic blood loss, childbirth, menstruation, gastrointestinal disorders (e.g., inflammatory bowel disease (IBD)), Chronic Kidney Disease (CKD), parasitic infections, insufficient dietary intake of iron, and insufficient absorption of iron.

There are typically three means by which IDA can be treated. The first approach is by eating foods that are high in iron. If that is insufficient, then a clinician may prescribe oral iron supplements. However, many oral iron supplements cause numerous adverse side effects in the patients, which leads to patient non-compliance. In those instances where an IDA patient cannot take oral iron supplements, he or she may have to have intravenous iron supplementation.

Intravenous (IV) iron supplementation is a method of delivering iron by injection with a needle, either through a muscle or into a vein. IDA patients who are receiving IV iron usually do so because they cannot tolerate oral iron. Intravenous iron is delivered into the IDA patient's vein through a needle that is attached to an IV bag that contains an iron solution. The procedure takes place in a doctor's office or a clinic and may take up to several hours, depending on which treatment the physician has prescribed. The patient usually receives iron injections over the course of several visits until his or her iron levels are correct. In some instances, an IDA patient may require chronic IV iron supplementation.

However, IV iron is also associated with short-term side effects such as gastrointestinal pains (e.g., nausea and cramps), breathing problems, skin problems (e.g., rash), chest pain, low blood pressure, anaphylaxis, and death, as well as long-term toxicity, including the development of atherosclerosis, infection, and increased mortality (Quinibi, Arzneimittelforschung (2010) 60, 399-412). Further, many clinics, particularly community sites, are ill-equipped to administer intravenous iron. This has left a majority of IDA patients without intravenous iron treatment.

IDA patients may also take one or more erythropoiesis-stimulating agents (ESAs) in an effort to control anemia. However, side effects can occur with ESA use. The most often side effects include: high blood pressure; swelling; fever; dizziness; nausea; and pain at the site of the injection, among others. In addition to these side effects, there are several safety issues that result from ESA use ESAs increase the risk of venous thromboembolism (blood clots in the veins). ESAs can also cause hemoglobin to rise too high, which puts the patient at higher risk for heart attack, stroke, heart failure, and death. In addition, ESAs may in certain cases worsen iron depletion and lead to an increase in thrombocytosis.

Thus, there is need to develop improved methods for oral iron treatment of IDA patients.

### 3. SUMMARY

In one aspect, provided herein are methods for treating iron-deficiency anemia (IDA) comprising administering ferric citrate or a pharmaceutical composition thereof to a subject in need thereof. See, e.g., Sections 4.2, infra, regarding the patient population treated, Section 4.3, infra, regarding the dosing and administration of ferric citrate or a pharmaceutical composition thereof, and Section 4.5, infra, regarding forms of ferric citrate and pharmaceutical compositions thereof. In one embodiment, provided herein is a method for treating iron deficiency anemia comprising orally administering a low dose of ferric citrate or a pharmaceutical composition thereof at a certain frequency (e.g., every day, every other day, every 2 days, every 3 days, every 4 days, every 5 days, etc. for a certain period of time) to a subject in need thereof. In particular embodiments, the low dose is administered once a day, every other day, or every two days for a period of time, such as 1 month, 2 months, 3 months, 4 months, 5 months, 6 months, 9 months, 12 months or more. In certain embodiments, the ferric citrate or pharmaceutical composition thereof is administered to a subject who has not ingested food within a certain timeframe. See, e.g., Section 4.3, infra, for examples of such timeframes for not having ingested food. In some embodiments, one or more iron storage parameters, such as hemoglobin concentration, transferrin saturation (TSAT) value, serum ferritin level, serum iron level, hematocrit level, total iron-binding capacity (TIBC) value, plasma erythropoietin level, and/or free erythrocyte protoporphyrin (FEP) level, of the subject are monitored (e.g., the one or more iron storage parameters are monitored every month, 2 months, 3 months, 4 months, 5 months, 6 months or more) and, in certain embodiments, the frequency of administration of ferric citrate or a pharmaceutical composition thereof and/or the amount of ferric citrate or a pharmaceutical composition thereof that the subject receives is altered based on the one or more iron storage parameters (e.g., the amount of ferric citrate or a pharmaceutical composition thereof is increased if the hemoglobin concentration has increased by less than 1 g/dl after a certain period of time, and the amount of ferric citrate or a pharmaceutical composition thereof is decreased if the hemoglobin concentration has increased by more than 5 g/dl, 4 g/dl, 3 g/dl, 2 g/dl or 1.5 g/dl). In certain embodiments, the subject administered the ferric citrate or pharmaceutical composition thereof does not have and/or has not been diagnosed with chronic kidney disease and/or hyperphosphatemia. In some embodiments, the patient has a gastrointestinal disorder, such as inflammatory bowel disease, inflammatory bowel syndrome, Crohn's disease, microscopic colitis (such as collagenous or lymphocytic colitis), and/or chemically-induced colitis (e.g., NSAID (nonsteroidal anti-inflammatory drug)-induced colitis). In certain embodiments, the patients treated for iron-deficiency anemia have blood loss (for example, blood loss associated with childbirth or menstruation, or blood loss associated with an infection). In some embodiments, the patients treated for iron-deficiency anemia have insufficient dietary intake of iron. In certain embodiments, the patients treated for iron-deficiency anemia have insufficient absorption of iron.

In a specific embodiment, provided herein is a method for treating iron deficiency anemia in a patient (e.g., a human patient), wherein the patient has not been diagnosed with chronic kidney disease, the method comprising orally administering a ferric citrate tablet containing approximately 210 mg of ferric iron to the patient, wherein the ferric citrate in the tablet is a complex of iron (+3), 0.70-0.87 (1, 2, 3-propanetricarboxylic acid, 2-hydroxy-), 1.9-3 (H₂O). In some embodiments, the patient has a serum ferritin level of between 5 ng/ml to 300 ng/ml (e.g., between 5 ng/ml to 250 ng/ml, between 5 ng/ml to 150 ng/ml, between 5 ng/ml to 100 ng/ml, between 5 ng/ml to 75 ng/ml, between 5 ng/ml to 50 ng/ml, between 5 ng/ml to 25 ng/ml, between 5 ng/ml to 15 ng/ml, or between 5 ng/ml to 10 ng/ml). In certain embodiments, the ferric citrate is not administered with food. In some embodiments, one or more iron storage parameters, such as hemoglobin concentration, TSAT value, serum ferritin level, serum iron level, hematocrit level, TIBC value, plasma erythropoietin level, and/or FEP level, of the subject are monitored (e.g., the one or more iron storage parameters are monitored every month, 2 months, 3 months, 4 months, 5 months, 6 months or more) and, in certain embodiments, the frequency of administration of ferric citrate or a pharmaceutical composition thereof and/or the amount of ferric citrate or a pharmaceutical composition thereof that the subject receives is altered based on the one or more iron storage parameters (e.g., the amount of ferric citrate or a pharmaceutical composition thereof is increased if the hemoglobin concentration has increased by less than 1 g/dl after a certain period of time, and the amount of ferric citrate or a pharmaceutical composition thereof is decreased if the hemoglobin concentration has increased by more than 5 g/dl, 4 g/dl, 3 g/dl, 2 &t or 1.5 g/dl). In certain embodiments, the patient has a gastrointestinal disorder, such as inflammatory bowel disuse, inflammatory bowel syndrome, Crohn's disease, ulcerative colitis, microscopic colitis (such as collagenous colitis or lymphocytic colitis), and/or chemically-induced colitis (e.g., NSAID-induced colitis). In certain embodiments, the patients treated for iron-deficiency anemia have blood loss (for example, blood loss associated with childbirth or menstruation, or blood loss associated with an infection). In some embodiments, the patients treated for iron-deficiency anemia have insufficient dietary intake of iron. In certain embodiments, the patients treated for iron-deficiency anemia have insufficient absorption of iron.

In another specific embodiment, provided herein is a method for treating iron deficiency anemia in a patient (e.g., a human patient), wherein the patient has not been diagnosed with chronic kidney disease and the patient has a serum ferritin level of between 5 ng/ml to 300 ng/ml (e.g., between 5 ng/ml to 250 ng/ml, between 5 ng/ml to 150 ng/ml, between 5 ng/ml to 100 ng/ml, between 5 ng/ml to 75 ng/ml, between 5 ng/ml to 50 ng/ml, between 5 ng/ml to 25 ng/ml, between 5 ng/ml to 15 ng/ml, or between 5 ng/ml to 10 ng/ml), the method comprising orally administering a ferric citrate tablet containing approximately 210 mg of ferric iron to the patient, wherein the ferric citrate is not administered within 2 hours of food being ingested by the patient, and wherein the ferric citrate in the tablet is a complex of iron (+3), 0.70-0.87 (1, 2, 3-propanetricarboxylic acid, 2-hydroxy-), 1.9-3 (H₂O). In some embodiments, one or more iron storage parameters, such as hemoglobin concentration, TSAT value, serum ferritin level, serum iron level, hematocrit level, TIBC value, plasma erythropoietin level, and/or FEP level, of the subject are monitored (e.g., the one or more iron storage parameters are monitored every month, 2 months, 3 months, 4 months, 5 months, 6 months or more) and, in certain embodiments, the frequency of administration of ferric citrate or a pharmaceutical composition thereof and/or the amount of ferric citrate or a pharmaceutical composition thereof that the subject receives is altered based on the one or more iron storage parameters (e.g., the amount of ferric citrate or a pharmaceutical composition thereof is increased if the hemoglobin concentration has increased by less than 1 g/dl after a certain period of time, and the amount of ferric citrate or a pharmaceutical composition thereof is decreased if the hemoglobin concentration has increased by more than 5 g/dl, 4 g/dl, 3 g/dl, 2 g/dl or 1.5 g/dl). In certain embodiments, the patient has a gastrointestinal disorder, such as inflammatory bowel disease, inflammatory bowel syndrome, Crohn's disease, ulcerative colitis, microscopic colitis (such as collagenous colitis or lymphocytic colitis), and/or chemically-induced colitis (e.g., NSAID-induced colitis). In certain embodiments, the patients treated for iron-deficiency anemia have blood loss (for example, blood loss associated with childbirth or menstruation, or blood loss associated with an infection). In some embodiments, the patients treated for iron-deficiency anemia have insufficient dietary intake of iron. In certain embodiments, the patients treated for iron-deficiency anemia have insufficient absorption of iron.

In another specific embodiment, provided herein is a method for treating iron deficiency anemia in a human patient that has not been diagnosed with chronic kidney disease, the method comprising: (a) orally administering to the patient one ferric citrate tablet containing approximately 210 mg of ferric iron per day, wherein the ferric citrate is not administered within 2 hours of food being ingested by the patient, and wherein the ferric citrate in the tablet is a complex of iron (+3), 0.70-0.87 (1, 2, 3-propanetricarboxylic acid, 2-hydroxy-), 1.9-3 (H₂O); and (b) decreasing the dose of ferric citrate after 4 weeks if the hemoglobin concentration of the subject has increased by more than 5 g/dl, 4 g/dl, 3 g/dl or 2 g/dl and increasing the dose of ferric citrate after 4 weeks if the hemoglobin concentration of the subject has increased by less than 1 g/dl. In some embodiments, one or more iron storage parameters, such as hemoglobin concentration, TSAT value, serum ferritin level, serum iron level, hematocrit level, TIBC value, plasma erythropoietin level, and/or FEP level, of the subject are monitored (e.g., the one or more iron storage parameters are monitored every month, 2 months, 3 months, 4 months, 5 months, 6 months or more). In certain embodiments, the patient has a gastrointestinal disorder, such as inflammatory bowel disuse, inflammatory bowel syndrome, Crohn's disease, ulcerative colitis, microscopic colitis (such as collagenous colitis or lymphocytic colitis), and/or chemically-induced colitis (e.g., NSAID-induced colitis). In certain embodiments, the patients treated for iron-deficiency anemia have blood loss (for example, blood loss associated with childbirth or menstruation, or blood loss associated with an infection). In some embodiments, the patients treated for iron-deficiency anemia have insufficient dietary intake of iron. In certain embodiments, the patients treated for iron-deficiency anemia have insufficient absorption of iron.

In a specific embodiment of any of the foregoing embodiments, the patients treated for iron-deficiency anemia are monitored for one or more iron storage parameters. The one or more iron storage parameters can be selected from the group consisting of hemoglobin concentration, serum ferritin level, TSAT value, serum iron level, hematocrit level, TIBC value, plasma erythropoietin level, and FEP level.

### 4. DETAILED DESCRIPTION

The present disclosure provides methods of using ferric citrate to treat a patient having iron-deficiency anemia (IDA). The present disclosure also provides pharmaceutical compositions, which may be administered to iron deficiency patients Methods of assessing patients before and/or after administering ferric citrate are also provided.

### 4.1. Methods for Treating IDA

In one aspect, provided herein are methods for treating IDA comprising administering ferric citrate or a pharmaceutical composition thereof to a subject in need thereof. In one embodiment, provided herein is a method for treating IDA comprising administering an effective amount of ferric citrate or a pharmaceutical composition thereof to a subject in need thereof. See, e.g., Sections 4.2, infra, regarding the patient population treated, Section 4.3, infra, regarding the dosing and administration of ferric citrate or a pharmaceutical composition thereof, and Section 4.5, infra, regarding forms of ferric citrate and pharmaceutical compositions thereof. In another embodiment, provided herein is a method for treating IDA comprising orally administering an effective amount of ferric citrate or a pharmaceutical composition thereof to a subject in need thereof. See, e.g., Sections 4.2, infra, regarding the patient population treated, Section 4.3, infra, regarding the dosing and administration of ferric citrate or a pharmaceutical composition thereof, and Section 4.5, infra, regarding forms of ferric citrate and pharmaceutical compositions thereof. In certain embodiments, one or more iron storage parameters, such as hemoglobin concentration, TSAT (transferring saturation) value, serum ferritin level, serum iron level, tissue iron level (e.g., stainable tissue iron level), hematocrit level, total iron-binding capacity (TIBC) value, plasma erythropoietin level, and/or free erythrocyte protoporphyrin (FEP) level, of the subject are assessed prior to administration of ferric citrate or a pharmaceutical composition thereof to the subject. In some embodiments, one or more iron storage parameters, such as hemoglobin concentration, TSAT value, serum ferritin level, serum iron level, tissue iron level (e.g., stainable tissue iron level), hematocrit level, TIBC value, plasma erythropoietin level, and/or FEP level, of the subject are monitored after the administration of ferric citrate or a pharmaceutical composition thereof to the subject (e.g., the one or more iron storage parameters are monitored every month, 2 months, 3 months, 4 months, 5 months, 6 months or more). In certain embodiments, the subject administered the ferric citrate or pharmaceutical composition thereof does not have and/or has not been diagnosed with chronic kidney disease and/or hyperphosphatemia.

In a specific embodiment, provided herein is a method for treating IDA comprising orally administering a low dose of ferric citrate or a pharmaceutical composition thereof at a certain frequency (e.g., every day, every other day, every 2 days, every 3 days, every 4 days, every 5 days, etc. for a certain period of time) to a subject in need thereof. In particular embodiments, the low dose is administered once a day, every other day, or every two days for a period of time, such as 1 month, 2 months, 3 months, 4 months, 5 months, 6 months, 9 months, 12 months or more. In certain embodiments, the ferric citrate or pharmaceutical composition thereof is administered to a subject who has not ingested food within a certain timeframe. See, e.g., Section 4.3, infra, for examples of such timeframes for not having ingested food. In some embodiments, one or more iron storage parameters, such as hemoglobin concentration, TSAT value, serum ferritin level, scrum iron level, tissue iron level (e.g., stainable tissue iron level), hematocrit level, TIBC value, plasma erythropoietin level, and/or FEP level, of the subject are monitored (e.g., the one or more iron storage parameters are monitored every month, 2 months, 3 months, 4 months, 5 months, 6 months or more) and, in certain embodiments, the frequency of administration of ferric citrate or a pharmaceutical composition thereof and/or the amount of ferric citrate or a pharmaceutical composition thereof that the subject receives is altered based on the one or more iron storage parameters (e.g., the amount of ferric citrate or a pharmaceutical composition thereof is increased if the hemoglobin concentration has increased by less than 1 g/dl after a certain period of time, and the amount of ferric citrate or a pharmaceutical composition thereof is decreased if the hemoglobin concentration has increased by more than 5 g/dl, 4 g/dl, 3 g/dl, 2 g/dl or 1.5 g/dl). In certain embodiments, the subject administered the ferric citrate or pharmaceutical composition thereof does not have and/or has not been diagnosed with chronic kidney disease and/or hyperphosphatemia.

As used herein, the term “low dose” in the context of ferric citrate or a pharmaceutical composition thereof is equivalent to a dose of 1100 mg of ferric iron or less but above 50 mg of ferric iron (in certain embodiments, above 100 mg or 200 mg of ferric iron). In one embodiment, a low dose of ferric citrate or a pharmaceutical composition thereof is equivalent to a dose of 1050 mg, 840 mg, 630 mg, 420 mg, or 210 mg of ferric iron. In another embodiment, a low dose of ferric citrate or a pharmaceutical composition thereof is equivalent to a dose of 1050 mg to 1100 mg, 840 mg to 1050 mg, 840 mg to 1100 mg, 630 mg to 840 mg, 630 mg to 1050 mg, 630 mg to 1100 mg, 420 mg to 630 mg, 420 mg to 840 mg, 420 mg to 1050 mg, 210 mg to 420 mg, 210 mg to 630 mg, 210 mg to 840 mg, or 210 mg to 1050 mg of ferric iron. In a specific embodiment, a low dose of ferric citrate or a pharmaceutical composition thereof is equivalent to 1, 2, 3, 4 or 5 tablets of Auryxia™ (Ferric Citrate; Keryx Biopharmaceuticals, inc.) per day or every other day.

In a specific embodiment, provided herein is a method for treating IDA comprising orally administering a low dose of ferric citrate or a pharmaceutical composition thereof at a certain frequency (e.g., every day, every other day, every 2 days, every 3 days, every 4 days, every 5 days, etc. for a certain period of time) to a subject in need thereof without food. In another specific embodiment, provided herein is a method for treating IDA comprising orally administering a low dose of ferric citrate or a pharmaceutical composition thereof at a certain frequency (e.g., every day, every other day, every 2 days, every 3 days, every 4 days, every 5 days, etc. for a certain period of time) to a subject in need thereof without food being ingested by the subject within 3 hours, 2 hours or 1 hour of ingestion of the ferric citrate or a pharmaceutical composition thereof. In particular embodiments, the low dose is administered once a day, every other day, or every two days for a period of time, such as 1 month, 2 months, 3 months, 4 months, 5 months, 6 months, 9 months, 12 months or more. In certain embodiments, one or more iron storage parameters, such as hemoglobin concentration, TSAT value, serum ferritin level, serum iron level, tissue iron level (e.g., stainable tissue iron level), hematocrit level, TIBC value, plasma erythropoietin level, and/or FEP level are assessed prior to administration of ferric citrate or a pharmaceutical composition thereof to the subject. In some embodiments, one or more iron storage parameters, such as hemoglobin concentration, TSAT value, serum ferritin level, serum iron level, tissue iron level (e.g., stainable tissue iron level), hematocrit level, TIBC value, plasma erythropoietin level, and/or FEP level, of the subject are monitored (e.g., the one or more iron storage parameters are monitored every month, 2 months, 3 months, 4 months, 5 months, 6 months or more) and, in certain embodiments, the frequency of administration of ferric citrate or a pharmaceutical composition thereof and/or the amount of ferric citrate or a pharmaceutical composition thereof that the subject receives is altered based on the one or more iron storage parameters (e.g., the amount of ferric citrate or a pharmaceutical composition thereof is increased if the hemoglobin concentration has increased by less than 1 g/dl after a certain period of time, and the amount of ferric citrate or a pharmaceutical composition thereof is decreased if the hemoglobin concentration has increased by more than 5 g/dl, 4 g/dl, 3 g/dl, 2 g/dl or 1.5 g/dl). In certain embodiments, the subject administered the ferric citrate or pharmaceutical composition thereof does not have and/or has not been diagnosed with chronic kidney disease and/or hyperphosphatemia.

In another embodiment, provided herein is a method for treating IDA in a subject, comprising: (a) assessing one or more of the following iron storage parameters: (1) the hemoglobin concentration, (ii) the TSAT value, (iii) the serum ferritin level, (iv) the serum iron level, (v) the tissue iron level (e.g., stainable tissue iron level), (vi) the hematocrit level, (vii) the TIBC value, (viii) the plasma erythropoictin level, and/or (ix) the FEP level of the subject; and (b) administering (e.g., orally administering) ferric citrate or a pharmaceutical composition thereof to a subject that has a certain hemoglobin concentration, TSAT value, serum ferritin level, serum iron level, tissue iron level (e.g., stainable tissue iron level), hematocrit level, TIBC value, plasma erythropoietin level, and/or FEP level. See, e.g., Section 4.2, infra, for hemoglobin concentrations, TSAT values, serum ferritin levels, serum iron levels, tissue iron levels (e.g., stainable tissue iron levels), hematocrit levels, TIBC values, plasma erythropoietin levels, and/or FEP levels of subjects that may be administered ferric citrate or a pharmaceutical composition in accordance with the methods described herein. In certain embodiments, a subject treated in accordance with the methods disclosed herein has one, two or all of the following prior to administration of ferric citrate or a pharmaceutical composition: (i) a hemoglobin concentration of approximately 6 grams/dl to approximately 8 grams/dl, approximately 6 grams/dl to approximately 10 grams/dl, approximately 6 grams/dl to approximately 12 grams/dl, approximately 7 grams/dl to approximately 9 grams/dl, approximately 7 grams/dl to approximately 11 grams/dl, approximately 7 grams/dl to approximately 13 grams/dl, approximately 8 grams/dl to approximately 10 grams/dl, approximately 8 grams/dl to approximately 12 grams/dl, approximately 9 grams/dl to approximately 11 grams/dl, approximately 9 grams/dl to approximately 12 grams/dl, approximately 9 grams/dl to approximately 13 grams/dl, approximately 10 grams/dl to approximately 11 grams/dl, approximately 10 grams/dl to approximately 12 grams/dl, approximately 10 grams/dl to approximately 13 grams/dl, approximately 11 grams/dl to approximately 12 grams/dl, approximately 11 grams/dl to approximately 13 grams/dl, or approximately 12 grams/dl to approximately 13 grams/dl; (ii) TSAI value of 10% to 45%, 12% to 45%, 20% to 45%, 20% to 40%, 10% to 35%, 20% to 25%, 15% to 50%, 10% to 30%, or 10% to 25%; (iii) a serum ferritin level of approximately 5 ng/ml to approximately 25 ng/ml, approximately 25 ng/ml to approximately 50 ng/ml, approximately 50 ng/ml to approximately 100 ng/ml, approximately 100 ng/ml to approximately 150 ng/ml, approximately 150 ng/ml to approximately 200 ng/ml, approximately 150 ng/ml to approximately 250 ng/ml, approximately 100 ng/ml to approximately 300 ng/ml, approximately 201) ng/ml to approximately 300 ng/ml, or approximately 250 ng/ml to approximately 300 ng/ml; (iv) serum iron level of approximately 10 μg/dl to approximately 20 μg/dl, approximately 10 μg/dl to approximately 30 μg/dl, approximately 10 μg/dl to approximately 40 μg/dl, approximately 10 μg/dl to approximately 50 μg/dl, approximately 10 μg/dl to approximately 60 μg/dl, approximately 20 μg/dl to approximately 30 μg/dl, approximately 20 μg/dl to approximately 40 μg/dl, approximately 20 μg/dl to approximately 50 μg/dl, approximately 20 μg/dl to approximately 60 μg/dl, approximately 30 μg/dl to approximately 40 μg/dl, approximately 30 μg/dl to approximately 50 μg/dl, approximately 30 μg/dl to approximately 60 μg/dl, approximately 40 μg/dl to approximately 50 μg/dl, or approximately 40 μg/dl to approximately 60 μg/dl; (v) tissue iron level (e.g., stainable tissue iron level) of grade 2, grade 1, or grade 0; (vi) hematocrit level of 10% to 15%, 10% to 20%, 10% to 25%, 10% to 30%, 10% to 35%, 10% to 40%, 10% to 45%, 15% to 20%, 15% to 25%, 15% to 30%, 15% to 35%, 15% to 40%, 15% to 45%, 20% to 25%, 20% to 30%, 20% to 35%, 20% to 40%, 25% to 45%, 25% to 30%, 25% to 35%, 25% to 40%, 25% to 45%, 30% to 35%, 30% to 40%, 30% to 45%, 35% to 40%, 35% to 45%, or 40% to 45%; (vii) TIBC value of approximately 390 μg/dl to approximately 600 μg/dl, approximately 390 μg/dl to approximately 800 μg/dl, approximately 390 μg/dl to approximately 1000 μg/dl, approximately 390 μg/dl to approximately 1200 μg/dl, approximately 500 μg/dl to approximately 700 μg/dl, approximately 500 μg/dl to approximately 900 μg/dl, approximately 500 μg/dl to approximately 1100 μg/dl, approximately 600 μg/dl to approximately 800 μg/dl, approximately 600 μg/dl to approximately 1000 μg/dl, approximately 600 μg/dl to approximately 1200 μg/dl, approximately 700 μg/dl to approximately 900 μg/dl, approximately 700 μg/dl to approximately 1100 μg/dl, approximately 800 μg/dl to approximately 1000 μg/dl, approximately 800 μg/dl to approximately 1200 μg/dl, approximately 900 μg/dl to approximately 1100 μg/dl, approximately 1000 μg/dl to approximately 1200 μg/dl; (viii) plasma erythropoietin level of approximately 20 mU/ml to approximately 30 mU/ml, approximately 20 mU/ml to approximately 40 mU/ml, approximately 20 mU/ml to approximately 50 mU/ml, approximately 20 mU/ml to approximately 60 mU/ml, approximately 30 mU/ml to approximately 40 mU/ml, approximately 30 mU/ml to approximately 50 mU/ml, approximately 30 mU/ml to approximately 60 mU/ml, approximately 40 mU/ml to approximately 50 mU/ml, approximately 40 mU/ml to approximately 60 mU/ml, or approximately 50 mU/ml to approximately 60 mU/ml; and/or (ix) PEP level of approximately 50 μg/dl to approximately 60 μg/dl, approximately 50 μg/dl to approximately 70 μg/dl, approximately 50 μg/dl to approximately 80 μg/dl, approximately 50 μg/dl to approximately 90 μg/dl, approximately 50 μg/dl to approximately 100 μg/dl, approximately 60 μg/dl to approximately 70 μg/dl, approximately 60 μg/dl to approximately 80 μg/dl, approximately 60 μg/dl to approximately 90 μg/dl, approximately 60 μg/dl to approximately 100 μg/dl, approximately 701401 to approximately 80 μg/dl, approximately 70 μg/dl to approximately 90 μg/dl, approximately 70 μg/dl to approximately 100 μg/dl, approximately 80 μg/dl to approximately 90 μg/dl, approximately 80 μg/dl to approximately 100 μg/dl, or approximately 90 μg/dl to approximately 100 μg/dl. In certain embodiments wherein the subject treated in accordance with the methods disclosed herein is a female, the subject has a TSAT value of 5% to 45%, 5% to 35%, 5% to 25%, 5% to 15%, 5% to 12%, 5% to 10%, 10% to 45%, 10% to 35%, 10% to 25%, 10% to 15%, 10% c to 12%, 12% to 45%, 12% to 35%, 12% to 25%, 12% to 15%, 20% to 45%, 20% to 35%, 20% to 25%, 30% to 45%, 30% to 35%, or 40% to 45% prior to administration of ferric citrate or a pharmaceutical composition thereof. In certain embodiments wherein the subject treated in accordance with the methods disclosed herein is a male, the subject has a TSAT value of 5% to 50%, 5% to 40%, 5% to 30%, 5% to 20%, 5% to 15%, 5% to 10%, 10% to 50%, 10% to 40%, 10% to 30%, 10% to 20%, 10% to 15%, 15% to 50%, 15% to 40%, 15% to 30%, 15% to 25%, 15% to 20%, 20% to 50%, 20% to 40%, 20% to 30%, 20% to 25%, 30% to 50%, 30% to 40%, 30% to 35%, 40% to 50%, 40% to 45%, or 45% to 50% prior to administration of ferric citrate or a pharmaceutical composition thereof. In a specific embodiment, the subject is administered a low dose of ferric citrate or a pharmaceutical composition thereof at a certain frequency (e.g., every day, every other day, every two days, every three days, every four days, or every five days). In another specific embodiment, the ferric citrate or pharmaceutical composition thereof is administered orally to the subject without food or not within a few hours, e.g., within less than 3 hours, of the ingestion of food by the subject. In some embodiments, the frequency of administration of ferric citrate or a pharmaceutical composition thereof and/or the amount of ferric citrate or a pharmaceutical composition thereof that the subject receives is altered based on the one or more iron storage parameters (e.g., the amount of ferric citrate or a pharmaceutical composition thereof is increased if the hemoglobin concentration has increased by less than 1 g/dl after a certain period of time, and the amount of ferric citrate or a pharmaceutical composition thereof is decreased if the hemoglobin concentration has increased by more than 5 g/dl, 4 g/dl, 3 g/dl, 2 g/dl or 1.5 g/dl). In certain embodiments, the subject administered the ferric citrate or pharmaceutical composition thereof does not have and/or has not been diagnosed with chronic kidney disease and/or hyperphosphatemia.

In another embodiment, provided herein is a method for treating IDA in a subject, comprising: (a) orally administering ferric citrate or a pharmaceutical composition thereof to a subject at a dose equivalent to 210 mg to 1100 mg of ferric iron per day or every other day; and (b) increasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, 5 months, 6 months, or more) if the hemoglobin concentration of the subject has increaser by less than 1 g/dl. In certain embodiments, the dose of ferric citrate or a pharmaceutical composition thereof is titrated up in increments, such as increments of 210 mg of ferric iron. In another embodiment, provided herein is a method for treating IDA in a subject, comprising: (a) orally administering ferric citrate or a pharmaceutical composition thereof to a subject at a dose equivalent to 210 mg of ferric iron per day or every other day; and (b) increasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, 5 months, 6 months, or more) if the hemoglobin concentration of the subject has increased by less than 1 g/dl. In certain embodiments, the dose is increased to 420 mg of ferric iron per day or every other day. In other embodiments, the dose is increased to 210 mg of ferric iron per day from 210 mg of ferric iron every other day. In a specific embodiment, the ferric citrate or pharmaceutical composition thereof is administered orally to the subject without food or not within a few hours, e.g., within less than 3 hours, of the ingestion of food by the subject. In certain embodiments, the subject administered the ferric citrate or pharmaceutical composition thereof does not have and/or has not been diagnosed with chronic kidney disease and/or hyperphosphatemia.

In another embodiment, provided herein is a method for treating IDA in a subject, comprising: (a) orally administering ferric citrate or a pharmaceutical composition thereof to a subject at a dose equivalent to 210 mg to 11.00 mg of ferric iron per day or every other day; (b) monitoring the subject after a certain period of time (e.g., 2 weeks, 4 weeks, 5 weeks, 6 weeks, 7 weeks, 8 weeks, 3 months, 4 months, 5 months, 6 months, or more); and (c) increasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, 5 months, 6 months, or more) if the hemoglobin concentration of the subject has increased by less than 1 g/dl. In certain embodiments, the dose of ferric citrate or a pharmaceutical composition thereof is titrated up in increments, such as increments of 210 mg of ferric iron. In another embodiment, provided herein is a method for treating IDA in a subject, comprising: (a) orally administering ferric citrate or a pharmaceutical composition thereof to a subject at a dose equivalent to 210 mg of ferric iron per day or every other day; (b) monitoring the subject after a certain period of time (e.g., 2 weeks, 4 weeks, 5 weeks, 6 weeks, 7 weeks, 8 weeks, 3 months, 4 months, 5 months, 6 months, or more); and (c) increasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, 5 months, 6 months, or more) if the hemoglobin concentration of the subject has increased by less than 1 g/dl. In certain embodiments, the dose is increased to 420 mg of ferric iron per day or every other day. In other embodiments, the dose is increased to 210 mg of ferric iron per day from 210 mg of ferric iron every other day. In a specific embodiment, the ferric citrate or pharmaceutical composition thereof is administered orally to the subject without food or not within a few hours, e.g., within less than 3 hours, of the ingestion of food by the subject. In certain embodiments, the subject administered the ferric citrate or pharmaceutical composition thereof does not have and/or has not been diagnosed with chronic kidney disease and/or hyperphosphatemia.

In another embodiment, provided herein is a method for treating IDA in a subject, comprising: (a) orally administering ferric citrate or a pharmaceutical composition thereof to a subject at a dose equivalent to 210 mg to 1100 mg of ferric iron per day or every other day; and (b) decreasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, 5 months, 6 months, or more) if the hemoglobin concentration of the subject has increased by more than 5 g/dl, 4 g/dl, 3 g/dl, 2 g/dl, or 1.5 g/dl. In certain embodiments, the dose of ferric citrate or a pharmaceutical composition thereof is titrated down in increments, such as increments of 210 mg of ferric iron. In another embodiment, provided herein is a method for treating IDA in a subject, comprising: (a) orally administering ferric citrate or a pharmaceutical composition thereof to a subject at a dose equivalent to 210 mg of ferric iron per day; and (b) decreasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, months, 6 months, or more) if the hemoglobin concentration of the subject has increased by more than 5 g/dl, 4 g/dl, 3 g/dl, 2 g/dl, or 1.5 g/dl. In certain embodiments, the dose is decreased 210 mg of ferric iron every other day from 210 mg of ferric iron per day. In a specific embodiment, the ferric citrate or pharmaceutical composition thereof is administered orally to the subject without food or not within a few hours, e.g., within less than 3 hours, of the ingestion of food by the subject. In certain embodiments, the subject administered the ferric citrate or pharmaceutical composition thereof does not have and/or has not been diagnosed with chronic kidney disease and/or hyperphosphatemia.

In another embodiment, provided herein is a method for treating IDA in a subject, comprising: (a) orally administering ferric citrate or a pharmaceutical composition thereof to a subject at a dose equivalent to 210 mg to 1100 mg of ferric iron per day or every other day; (b) monitoring the subject after a certain period of time (e.g., 2 weeks, 4 weeks, 5 weeks, 6 weeks, 7 weeks, 8 weeks, 3 months, 4 months, 5 months, 6 months, or more); and (c) decreasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, 5 months, 6 months, or more) if the hemoglobin concentration of the subject has increased by more than 5 g/dl, 4 g/dl, 3 g/dl, 2 g/dl, or 1.5 g/dl. In certain embodiments, the dose of ferric citrate or a pharmaceutical composition thereof is titrated down in increments, such as increments of 210 mg of ferric iron. In another embodiment, provided herein is a method for treating IDA in a subject, comprising: (a) orally administering ferric citrate or a pharmaceutical composition thereof to a subject at a dose equivalent to 210 mg of ferric iron per day; (b) monitoring the subject after a certain period of time (e.g., 2 weeks, 4 weeks, 5 weeks, 6 weeks, 7 weeks, 8 weeks, 3 months, 4 months, 5 months, 6 months, or more); and (c) decreasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, 5 months, 6 months, or more) if the hemoglobin concentration of the subject has increased by more than 5 g/dl, 4 g/dl, 3 g/dl, 2 g/dl or 1.5 g/dl. In certain embodiments, the dose is decreased to 210 mg of ferric iron every other day from 210 mg of ferric iron per day. In a specific embodiment, the ferric citrate or pharmaceutical composition thereof is administered orally to the subject without food or not within a few hours, e.g., within less than 3 hours, of the ingestion of food by the subject. In certain embodiments, the subject administered the ferric citrate or pharmaceutical composition thereof does not have and/or has not been diagnosed with chronic kidney disease and/or hyperphosphatemia.

In another embodiment, provided herein is a method for treating IDA in a subject, comprising: (a) orally administering ferric citrate or a pharmaceutical composition thereof to a subject at a dose equivalent to 210 mg to 1100 mg of ferric iron per day or every other day; and (b) decreasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, months, 6 months, or more) if the hemoglobin concentration of the subject has increased by more than 5 g/dl, 4 g/dl, 3 g/dl, 2 g/dl, or 1.5 g/dl and increasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, 5 months, 6 months, or more) if the hemoglobin concentration of the subject has increased by less than 1 g/dl. In certain embodiments, the dose of ferric citrate or a pharmaceutical composition thereof is titrated down or up in increments, such as increments of 210 mg of ferric iron. In another embodiment, provided herein is a method for treating IDA in a subject, comprising: (a) orally administering ferric citrate or a pharmaceutical composition thereof to a subject at a dose equivalent to 210 mg of ferric iron per day; and (b) decreasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, 5 months, 6 months, or more) if the hemoglobin concentration of the subject has increased by more than 5 g/dl, 4 g/dl, 3 g/dl, 2 g/dl, or 1.5 g/dl and increasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, 5 months, 6 months, or more) if the hemoglobin concentration of the subject has increased by less than 1 g/dl. In certain embodiments, the dose is decreased to 210 mg of ferric iron every other day from 210 mg of ferric iron per day. In other embodiments, the dose is increased to 420 mg of ferric iron per day or every other day. In a specific embodiment, the ferric citrate or pharmaceutical composition thereof is administered orally to the subject without food or not within a few hours, e.g., within less than 3 hours, of the ingestion of food by the subject. In certain embodiments, the subject administered the ferric citrate or pharmaceutical composition thereof does not have and/or has not been diagnosed with chronic kidney disease and/or hyperphosphatemia.

In another embodiment, provided herein is a method for treating IDA in a subject, comprising: (a) orally administering ferric citrate or a pharmaceutical composition thereof to a subject at a dose equivalent to 210 mg to 1100 mg of ferric iron per day; (b) monitoring the subject after a certain period of time (e.g., 2 weeks, 4 weeks, 5 weeks, 6 weeks, 7 weeks, 8 weeks, 3 months, 4 months, 5 months, 6 months, or more); and (c) decreasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, 5 months, 6 months, or more) if the hemoglobin concentration of the subject has increased by more than 5 g/dl, 4 g/dl, 3 g/dl, 2 g/dl, or 1.5 g/dl and increasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, 5 months, 6 months, or more) if the hemoglobin concentration of the subject has increased by less than 1 g/dl. In certain embodiments, the dose of ferric citrate or a pharmaceutical composition thereof is titrated down or up in increments, such as increments of 210 mg of ferric iron. In another embodiment, provided herein is a method for treating IDA in a subject, comprising: (a) orally administering ferric citrate or a pharmaceutical composition thereof to a subject at a dose equivalent to 210 mg of ferric iron per day; (h) monitoring the subject after a certain period of time (e.g., 2 weeks, 4 weeks, 5 weeks, 6 weeks, 7 weeks, 8 weeks, 3 months, 4 months, 5 months, 6 months, or more), and (c) decreasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, 5 months, 6 months, or more) if the hemoglobin concentration of the subject has increased by more than 5 g/dl, 4 g/dl, 3 g/dl, 2 g/dl, or 1.5 g/dl and increasing the dose of ferric citrate or a pharmaceutical composition thereof after a certain period of time (e.g., 2 weeks, 4 weeks, 5 weeks, 6 weeks, 7 weeks, 8 weeks, 3 months, 4 months, 5 months, 6 months, or more) if the hemoglobin concentration of the subject has increased by less than 1 g/dl. In certain embodiments, the dose is decreased to 210 mg of ferric iron every other day from 210 mg of ferric iron per day. In other embodiments, the dose is increased to 420 mg of ferric iron per day or every other day. In a specific embodiment, the ferric citrate or pharmaceutical composition thereof is administered orally to the subject without food or not within a few hours, e.g., within less than 3 hours, of the ingestion of food by the subject. In certain embodiments, the subject administered the ferric citrate or pharmaceutical composition thereof does not have and/or has not been diagnosed with chronic kidney disease and/or hyperphosphatemia.

In certain embodiments, a subject treated for IDA in accordance with the methods described herein experiences a therapeutic benefit. In specific embodiments, a subject treated for IDA in accordance with the methods described herein experiences one, two, three or more, or all of the following effects: (i) an improvement in one or more symptoms of IDA; (ii) a reduction in the number of symptoms associated with IDA; (iii) a reduction in the duration of one or more symptoms; (iv) an improvement (e.g., an increase) in one or more iron storage parameters, such as hemoglobin concentration, TSAT value, serum ferritin level, serum iron level, tissue iron level (e.g., stainable tissue iron level), hematocrit level, TIBC value, plasma erythropoietin level, and/or FEP level; (v) a reduction in the administration of intravenous iron and/or an erythropoiesis stimulating agent; (vi) a decrease in iron deficiency; and/or (vii) a decrease or elimination of one, two, three, four or more symptoms of IDA. Symptoms of IDA include, but are not limited to, fatigue, dizziness, lightheadedness, pallor, hair loss, irritability, weakness, pica, brittle or grooved nails, dyspnea, anxiety, sadness, angina, constipation, sleepiness, tinnitus, mouth ulcers, Plummer-Vinson syndrome (painful atrophy of the mucous membrane covering the tongue, pharynx and esophagus), palpitations, hair loss, fainting or feeling faint, depression, twitching muscles, pale yellow skin, tingling (numbness) or burning sensations, missed menstrual cycle(s), heavy menstrual period(s), slow social development, glossitis, angular cheilitis, koilonychias, poor appetite, prurius, insomnia, dizziness, strange cravings for non-food items (e.g., dirt, ice, and clay), fast or irregular heartbeat, headaches, shortness of breath, cold hands and feet, impaired immune function, pagophagia, restless legs syndrome and combinations of the foregoing. In certain embodiments, a decrease in iron deficiency occurs as the total amount of iron in the body of the IDA patient is increased through the administration of the ferric citrate or a pharmaceutical composition thereof.

In specific aspects, provided herein are methods for increasing iron absorption in a subject with and/or diagnosed with IDA, comprising orally administering ferric citrate or a pharmaceutical composition thereof to the subject. See, e.g., Sections 4.2, infra, regarding the patient population treated, Section 4.3, infra, regarding the dosing and administration of ferric citrate or a pharmaceutical composition thereof, and Section 4.5, infra, regarding forms of ferric citrate and pharmaceutical compositions thereof. In specific embodiments, the subject is administered a low dose of ferric citrate at a certain frequency (e.g., every day, every other day, every 2 days, every 3 days, every 4 days, or every 5 days). In certain embodiments, one or more iron storage parameters, such as hemoglobin concentration, TSAT value, serum ferritin level, serum iron level, tissue iron level (e.g., stainable tissue iron level), hematocrit level, TIBC value, plasma erythropoietin level, and/or FEP leve, of the subject are assessed prior to administration of ferric citrate or a pharmaceutical composition thereof to the subject. In some embodiments, one or more iron storage parameters, such as hemoglobin concentration, TSAT value, serum ferritin level, serum iron level, tissue iron level (e.g., stainable tissue iron level), hematocrit level, TIBC value, plasma erythropoietin level, and/or FEP level, of the subject are monitored after the administration of ferric citrate or a pharmaceutical composition thereof to the subject (e.g., the one or more iron storage parameters are monitored every month, 2 months, 3 months, 4 months, 5 months, 6 months or more). In certain embodiments, the subject administered the ferric citrate or pharmaceutical composition thereof does not have and/or has not been diagnosed with chronic kidney disease and/or hyperphosphatemia.

In specific aspects, provided herein are methods for maintaining or increasing iron stores in a subject with and/or diagnosed with IDA, comprising orally administering ferric citrate or a pharmaceutical composition thereof to the subject. See, e.g., Sections 4.2, infra, regarding the patient population treated, Section 4.3, infra, regarding the dosing and administration of ferric citrate or a pharmaceutical composition thereof, and Section 4.5, infra, regarding forms of ferric citrate and pharmaceutical compositions thereof. In specific embodiments, the subject is administered a low dose of ferric citrate at a certain frequency (e.g., every day, every other day, every 2 days, every 3 days, every 4 days, or every 5 days). There are several markers of systemic iron status that may be measured to determine whether an IDA patient has sufficient iron stores to maintain adequate health. These markers may be of circulating iron stores, iron stored in iron-binding complexes, or both, and are also typically referred to as iron storage parameters. Iron storage parameters can include, for example, hematocrit, hemoglobin concentration (Hb), total iron-binding capacity (TIBC), TSAT, serum iron level, tissue iron level (e.g., liver iron level, spleen iron level) measured as stainable tissue iron level or tissue iron concentration, serum ferritin level, plasma erythropoietin level, and FEP level. Of these, the hematocrit, hemoglobin concentration (Hb), total iron-binding capacity (TIBC), TSAT and serum iron level are commonly known as circulating iron stores. The liver iron level, spleen iron level, and serum ferritin level are commonly referred to as stored iron or iron stored in iron-binding complexes. In certain embodiments, one or more iron storage parameters, such as hemoglobin concentration, TSAT value, serum ferritin level, serum iron level, tissue iron level (e.g., stainable tissue iron level), hematocrit level, TIBC value, plasma erythropoietin level, and/or FEP level, of the subject are assessed prior to administration of ferric citrate or a pharmaceutical composition thereof to the subject. In some embodiments, one or more iron storage parameters, such as hemoglobin concentration, TSAT value, serum ferritin level, serum iron level, tissue iron level (e.g., stainable tissue iron level), hematocrit level, TIBC value, plasma erythropoietin level, and/or FEP level, of the subject are monitored after the administration of ferric citrate or a pharmaceutical composition thereof to the subject (e.g., the one or more iron storage parameters are monitored every month, 2 months, 3 months, 4 months, 5 months, 6 months or more). In certain embodiments, the subject administered the ferric citrate or pharmaceutical composition thereof does not have and/or has not been diagnosed with chronic kidney disease and/or hyperphosphatemia.

In specific aspects, provided herein are methods for improving one or more iron storage parameters in a subject with and/or diagnosed with IDA, comprising orally administering ferric citrate or a pharmaceutical composition thereof to the subject. See, e.g., Sections 4.2, infra, regarding the patient population treated, Section 4.3, infra, regarding the dosing and administration of ferric citrate or a pharmaceutical composition thereof, and Section 4.5, infra, regarding forms of ferric citrate and pharmaceutical compositions thereof. In some embodiments, the one or more iron storage parameters are selected from hematocrit, hemoglobin concentration (Hb), total iron-binding capacity (TIBC), TSAT, serum iron level, tissue iron level (e.g., liver iron level, spleen iron level) measured as stainable tissue iron level or tissue iron concentration, serum ferritin level, plasma erythropoietin level and FEP level. In specific embodiments, the subject is administered a low dose of ferric citrate at a certain frequency (e.g., every day, every other day, every 2 days, every 3 days, every 4 days, or every 5 days). In certain embodiments, one or more iron storage parameters, such as hemoglobin concentration, TSAT value, serum ferritin level, serum iron level, tissue iron level (e.g., stainable tissue iron level), hematocrit level, TIBC value, plasma erythropoietin level, and/or FEP level, of the subject are assessed prior to administration of ferric citrate or a pharmaceutical composition thereof to the subject. In some embodiments, one or more iron storage parameters, such as hemoglobin concentration, TSAT value, serum ferritin level, serum iron level, tissue iron level (e.g., stainable tissue iron level), hematocrit level, TIBC value, plasma erythropoietin level, and/or FEP level, of the subject are monitored after the administration of ferric citrate or a pharmaceutical composition thereof to the subject (e.g., the one or more iron storage parameters are monitored every month, 2 months, 3 months, 4 months, 5 months, 6 months or more). In certain embodiments, the subject administered the ferric citrate or pharmaceutical composition thereof does not have and/or has not been diagnosed with chronic kidney disease and/or hyperphosphatemia.

In specific aspects, provided herein are methods for increasing or maintaining serum ferritin level in a subject with and/or diagnosed with IDA, comprising orally administering ferric citrate or a pharmaceutical composition thereof to the subject. See, e.g., Sections 4.2, infra, regarding the patient population treated, Section 4.3, infra, regarding the dosing and administration of ferric citrate or a pharmaceutical composition thereof, and Section 4.5, infra, regarding forms of ferric citrate and pharmaceutical compositions thereof. In specific embodiments, the subject is administered a low dose of ferric citrate at a certain frequency (e.g., every day, every other day, every 2 days, every 3 days, every 4 days, or every 5 days). In certain embodiments, the serum ferritin level of the subject is assessed prior to administration of ferric citrate or a pharmaceutical composition thereof to the subject. In some embodiments, the serum ferritin level of the subject is monitored after the administration of ferric citrate or a pharmaceutical composition thereof to the subject (e.g., monitored every month, 2 months, 3 months, 4 months, 5 months, 6 months or more). In certain embodiments, one or more other iron storage parameters, such as hemoglobin concentration, TSAT value, serum iron level, tissue iron level (e.g., stainable tissue iron level), hematocrit level, TIBC value, plasma erythropoietin level, and/or FEP level, of the subject are assessed prior to administration of ferric citrate or a pharmaceutical composition thereof to the subject. In some embodiments, one or more other iron storage parameters, such as hemoglobin concentration, TSAT value, serum iron level, tissue iron level (e.g., stainable tissue iron level), hematocrit level, TIBC value, plasma erythropoietin level, and/or FEP level, of the subject are monitored after the administration of ferric citrate or a pharmaceutical composition thereof to the subject (e.g., the one or more iron storage parameters are monitored every month, 2 months, 3 months, 4 months, 5 months, 6 months or more). In certain embodiments, the subject administered the ferric citrate or pharmaceutical composition thereof does not have and/or has not been diagnosed with chronic kidney disease and/or hyperphosphatemia.

The liver's stores of ferritin are the primary source of stored iron in the body. Ferritin is an intracellular protein that stores iron and releases it in a controlled fashion. Medically, the amount of ferritin present in a blood sample and/or in a sample of liver tissue reflects the amount of iron that is stored in the liver (although ferritin is ubiquitous and can be found in many other tissues within the body in addition to the liver). Ferritin serves to store iron in the liver in a non-toxic form and to transport it to areas where it is required. A normal ferritin blood serum level, sometimes referred to as the reference interval, is usually between 30-300 ng/ml for males, and 15-200 ng/ml for females. In an IDA patient, however, serum ferritin levels are typically markedly reduced as the amount of iron available to be bound by ferritin and stored in the liver is decreased, which occurs as the body loses its ability to absorb and/or store iron.

In certain embodiments, a subject treated for IDA in accordance with the methods described herein experiences mean increase in serum ferritin level of 5-15 ng/ml, 5-25 ng/ml, 5-50 ng/ml, 5-100 ng/ml, 5-200 ng/ml, 5-300 ng/ml, 5-400 ng/ml, 25-50 ng/ml, 25-100 ng/ml, 25-200 ng/ml, 25-300 ng/ml, 25-400 ng/ml, 50-100 ng/ml, 50-200 ng/ml, 50-300 ng/ml, 50-400 ng/ml, 100-200 ng/ml, 100-300 ng/ml, 100-400 ng/ml, 200-300 ng/ml, or 200-400 ng/ml. In some embodiments, a subject treated for IDA in accordance with the methods described herein experiences mean increase in serum ferritin level of about 5 ng/ml or more, about 10 ng/ml or more, about 25 ng/ml or more, about 50 ng/ml or more, about 100 ng/ml or more, about 110 ng/ml or more, about 120 ng/ml or more, about 130 ng/ml or more, about 140 ng/ml or more, about 150 ng/ml or more, about 160 ng/ml or more, about 170 ng/ml or more, about 180 ng/ml or more, about 190 ng/ml or more, about 200 ng/ml or more, about 210 ng/ml or more, about 220 ng/ml or more, about 230 ng/ml or more, about 240 ng/ml or more, about 250 ng/ml or more, about 260 ng/ml or more, about 270 ng/ml or more, about 280 ng/ml or more, about 290 ng/ml or more, about 300 ng/ml or more, about 310 ng/ml or more, about 320 ng/ml or more, about 330 ng/ml or more, about 340 ng/ml or more, about 350 ng/ml or more, about 360 ng/ml or more, about 370 ng/ml or more, about 380 ng/ml or more, or about 390 ng/ml or more. In certain embodiments, a subject treated for IDA in accordance with the methods described herein experiences mean increase in serum ferritin level of about 1-100%, 1-95%, 10-95%, 10-90%, 10-85%, 10-80%, 10-75%, 10-70%, 10-65%, 10-60%, 10-50%, 10-45%, 10-40%, 10-35%, 10-30%, 10-25%, 10-20%, 20-30%, 20-40%, 20-50%, 20-60%, 20-70%, 20-80%, 20-90%, 30-90%, 30-80%, 30-70%, 30-60%, 30-50%, 30-40%, 40-90%, 40-80%, 40-70%, 40-60%, 40-50%, 50-90%, 50-80%, 50-70%, 50-65%, 50-60%, 60-90%, 60-80%, 60-75%, 60-70%, 70-90%, 70%-80%, or 80-90%. In some embodiments, a subject treated for IDA in accordance with the methods described herein experiences mean increase in serum ferritin level of 10%, 15%, 204%, 25%, 30%, 35%, 40%, 45%, 50%, 55%, 60%, 65%, 70%, 75%, 80%, 85%, 90%, 95% or more. In certain embodiments, a mean increase of serum ferritin level results after the ferric citrate or a pharmaceutical composition thereof is administered to the subject for a certain period of time (e.g., 1 month, 2 months, 3 months, 4 months, 5 months, 6 months, 7 months, 8 months, 9 months, 10 months, 11 months, 12 months or more). In some embodiments, a subject treated for IDA in accordance with the methods described herein experiences maintenance of their serum ferritin level such that their serum ferritin level remains substantially unchanged during administration of the ferric citrate or a pharmaceutical composition.

As used herein, the term “substantially unchanged” in the context of the level of an iron storage parameter, means that the level of the iron storage parameter is changed less than 5%.

In specific aspects, provided herein are methods for increasing or maintaining tissue iron level (e.g., liver iron level, spleen iron level) measured as stainable tissue iron levels or tissue iron concentrations in a subject with and/or diagnosed with IDA, comprising orally administering ferric citrate or a pharmaceutical composition thereof to the subject. In a specific embodiment, the tissue iron level is measured as stainable tissue iron level. See, e.g., Sections 4.2, infra, regarding the patient population treated, Section 4.3, infra, regarding the dosing and administration of ferric citrate or a pharmaceutical composition thereof, and Section 4.5, infra, regarding forms of ferric citrate and pharmaceutical compositions thereof. In specific embodiments, the subject is administered a low dose of ferric citrate at a certain frequency (e.g., every day, every other day, every 2 days, every 3 days, every 4 days, or every 5 days). In certain embodiments, the tissue iron level (e.g., stainable tissue iron level) of the subject is assessed prior to administration of ferric citrate or a pharmaceutical composition thereof to the subject. In some embodiments, the tissue iron level (e.g., stainable tissue iron level) of the subject is monitored after the administration of ferric citrate or a pharmaceutical composition thereof to the subject (e.g., monitored every month, 2 months, 3 months, 4 months, 5 months, 6 months or more). In certain embodiments, one or more other iron storage parameters, such as hemoglobin concentration, TSAT value, serum ferritin level, serum iron level, hematocrit level, TIBC value, plasma erythropoietin level, and/or FEP level, of the subject are assessed prior to administration of ferric citrate or a pharmaceutical composition thereof to the subject. In some embodiments, one or more other iron storage parameters, such as hemoglobin concentration, TSAT value, serum ferritin level, serum iron level, hematocrit level, TIBC value, plasma erythropoietin level, and/or FEP level, of the subject are monitored after the administration of ferric citrate or a pharmaceutical composition thereof to the subject (e.g., the one or more iron storage parameters are monitored every month, 2 months, 3 months, 4 months, 5 months, 6 months or more). In certain embodiments, the subject administered the ferric citrate or pharmaceutical composition thereof does not have and/or has not been diagnosed with chronic kidney disease and/or hyperphosphatemia.

Tissue iron levels reflect the iron content in tissues (e.g., liver, spleen), and can be measured as stainable tissue iron levels or tissue iron concentrations. Stainable tissue iron levels and serum ferritin levels are the most sensitive laboratory indicators of mild iron deficiency and are particularly useful in differentiating iron deficiency from the anemia of chronic disorders. Stainable tissue iron levels are determined by histological grading of stainable iron. A normal stainable liver iron level is usually greater than grade 3. In an IDA patient, however, the stainable liver iron level is typically markedly reduced as the body loses its ability to absorb and/or store iron.

In certain embodiments, a subject treated for IDA in accordance with the methods described herein experiences mean increase in tissue iron level (e.g., stainable tissue iron level) of about 1-100%, 1-95%, 10-95%, 10-90%, 10-85%, 10-80%, 10-75%, 10-70%, 10-65%, 10-60%, 10-50%, 10-45%, 10-40%, 10-35%, 10-30%, 10-25%, 10-20%, 20-30%, 20-40%, 20-50%, 20-60%, 20-70%, 20-80%, 20-90%, 30-90%, 30-80%, 30-70%, 30-60%, 30-50%, 30-40%, 40-90%, 40-80%, 40-70%, 40-60%, 40-50%, 50-90%, 50-80%, 50-70%, 50-65%, 50-60%, 60-90%, 60-80%, 60-75%, 60-70%, 70-90%, 70%-80%, or 80-90%. In some embodiments, a subject treated for IDA in accordance with the methods described herein experiences mean increase in tissue iron level (e.g., stainable tissue iron level) of 10%, 15%, 20%, 25%, 30%, 35%, 40%, 45%, 50%, 55%, 60%, 65%, 70%, 75%, 80%, 85%, 90%, 95% or more. In certain embodiments, a mean increase of tissue iron level (e.g., stainable tissue iron level) results after the ferric citrate or a pharmaceutical composition thereof is administered to the subject for a certain period of time (e.g., 1 month, 2 months, 3 months, 4 months, 5 months, 6 months, 7 months, 8 months, 9 months, 10 months, 11 months, 12 months or more). In some embodiments, a subject treated for IDA in accordance with the methods described herein experiences maintenance of their tissue iron level (e.g., stainable tissue iron level) such that their tissue iron level (e.g., stainable tissue iron level) remains substantially unchanged during administration of the ferric citrate or a pharmaceutical composition.

In specific aspects, provided herein are methods for increasing or maintaining TSAT value in a subject with and/or diagnosed with IDA, comprising orally administering ferric citrate or a pharmaceutical composition thereof to the subject. See, e.g., Sections 4.2, infra, regarding the patient population treated, Section 4.3, infra, regarding the dosing and administration of ferric citrate or a pharmaceutical composition thereof, and Section 4.5, infra, regarding forms of ferric citrate and pharmaceutical compositions thereof. In specific embodiments, the subject is administered a low dose of ferric citrate at a certain frequency (e.g., every day, every other day, every 2 days, every 3 days, every 4 days, or every 5 days). In certain embodiments, the TSAT value of the subject is assessed prior to administration of ferric citrate or a pharmaceutical composition thereof to the subject. In some embodiments, the TSAT value of the subject is monitored after the administration of ferric citrate or a pharmaceutical composition thereof to the subject (e.g., monitored every month, 2 months, 3 months, 4 months, 5 months, 6 months or more). In certain embodiments, one or more other iron storage parameters, such as hemoglobin concentration, serum ferritin level, serum iron level, tissue iron level (e.g., stainable tissue iron level), hematocrit level, TIBC value, plasma erythropoietin level, and/or FEP level, of the subject are assessed prior to administration of ferric citrate or a pharmaceutical composition thereof to the subject. In some embodiments, one or more other iron storage parameters, such as hemoglobin concentration, serum ferritin level, serum iron level, tissue iron level (e.g., stainable tissue iron level), hematocrit level, TIBC value, plasma erythropoietin level, and/or FEY, level, of the subject are monitored after the administration of ferric citrate or a pharmaceutical composition thereof to the subject (e.g., the one or more iron storage parameters are monitored every month, 2 months, 3 months, 4 months, 5 months, 6 months or more). In certain embodiments, the subject administered the ferric citrate or pharmaceutical composition thereof does not have and/or has not been diagnosed with chronic kidney disease and/or hyperphosphatemia.

In addition to stored iron, a small amount of iron, typically about 3 to 4 mg, circulates through the blood plasma bound to a protein called transferrin. Therefore, serum iron levels can be represented by the amount of iron circulating in the blood that is bound to the protein transferrin. Transferrin is a glycoprotein produced by the liver that can bind one or two ferric iron (iron(III) or Fe3+) ions. It is the most prevalent and dynamic carrier of iron in the blood, and therefore is an essential component of the body's ability to transport stored iron for use throughout the body. Transferrin saturation (or TSAT) is measured as a percentage and is calculated as the ratio of serum iron and total iron binding capacity, multiplied by 100. This value tells a clinician how much serum iron is actually bound to the total amount of transferrin that is available to bind iron. For instance, a TSAT value of 35% means that 35% of the available iron-binding sites of transferrin in a blood sample is occupied by iron. In non-IDA patients, typical TSAT values are approximately 15-50% for males and 12-45% for females. In an IDA patient, however. TSAT values are typically markedly reduced as the amount of iron available to be bound by transferrin is decreased, which occurs as the body loses its ability to absorb and/or store iron.

In certain embodiments, a subject treated for IDA in accordance with the methods described herein experiences mean increase in TSAT value of about 1-10%, 1-15%, 1-20%, 1-25%, 1-50%, 1-75%, 1-100%, 5-15%, 5-20%, 5-25%, 5-50%, 5-75%, 5-100%, 10-15%, 10-20%, 10-25%, 10-50%, 10-75%, 10-100%, 15-20%, 15-25%, 15-50%, 15-75%, 15-100%, 20-25%, 20-50%, 20-75%, 20-100%, 25-50%, 25-75%, 25-100%, 50-75%, or 50-100%. In some embodiments, a subject treated for IDA in accordance with the methods described herein experiences mean increase in TSAT values of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 50%, 75%, 100% or more. In certain embodiments, a mean increase of TSAT value results after the ferric citrate or a pharmaceutical composition thereof is administered to the subject for a certain period of time (e.g., 1 month, 2 months, 3 months, 4 months, 5 months, 6 months, 7 months, 8 months, 9 months, 10 months, 11 months, 12 months or more). In some embodiments, a subject treated for IDA in accordance with the methods described herein experiences maintenance of their TSAT value such that their TSAT value remains substantially unchanged during administration of the ferric citrate or a pharmaceutical composition.

In specific aspects, provided herein are methods for increasing or maintaining hemoglobin concentration in a subject with and/or diagnosed with IDA, comprising orally administering ferric citrate or a pharmaceutical composition thereof to the subject. See, e.g., Sections 4.2, infra, regarding the patient population treated, Section 4.3, infra, regarding the dosing and administration of ferric citrate or a pharmaceutical composition thereof, and Section 4.5, Infra, regarding forms of ferric citrate and pharmaceutical compositions thereof. In specific embodiments, the subject is administered a low dose of ferric citrate at a certain frequency (e.g., every day, every other day, every 2 days, every 3 days, every 4 days, or every 5 days). In certain embodiments, the hemoglobin concentration of the subject is assessed prior to administration of ferric citrate or a pharmaceutical composition thereof to the subject. In some embodiments, the hemoglobin concentration of the subject is monitored after the administration of ferric citrate or a pharmaceutical composition thereof to the subject (e.g., monitored every month, 2 months, 3 months, 4 months, 5 months, 6 months or more). In certain embodiments, one or more other iron storage parameters, such as TSAT value, serum ferritin level, serum iron level, tissue iron level (e.g., stainable tissue iron level), hematocrit level, TIBC value, plasma erythropoietin level, and/or FEP level, of the subject are assessed prior to administration of ferric citrate or a pharmaceutical composition thereof to the subject. In some embodiments, one or more other iron storage parameters, such as TSAT value, serum ferritin level, serum iron level, tissue iron level (e.g., stainable tissue iron level), hematocrit level, TIBC value, plasma erythropoietin level, and/or FEP level, of the subject are monitored after the administration of ferric citrate or a pharmaceutical composition thereof to the subject (e.g., the one or more iron storage parameters are monitored every month, 2 months, 3 months, 4 months, 5 months, 6 months or more). In certain embodiments, the subject administered the ferric citrate or pharmaceutical composition thereof does not have and/or has not been diagnosed with chronic kidney disease and/or hyperphosphatemia.

Hemoglobin concentration is the measure of the concentration of hemoglobin (grams) per volume (deciliter) of whole blood. Hemoglobin concentration may also be measured as a mass or weight fraction and presented as a percentage (5). For non-IDA patients, a typical hemoglobin concentration ranges from 13.8-18.0 g/dl (i.e., 8.56-11.17 mmol/L) for men, from 12.1-15.1 g/dl (i.e., 7.51-9.37 mmol/L) for women, from 11.0-16.0 g/dl (i.e., 6.83-9.93 mmol/L) for children, and from 11.0-14.0 g/dl (i.e., 6.83-8.69 mmol/L) for pregnant women. In an IDA patient, however, the hemoglobin concentration can be reduced below the normal range as the body loses its ability to absorb and/or store iron.

In certain embodiments, a subject treated for IDA in accordance with the methods described herein experiences mean increase in hemoglobin concentration of 0.1-0.5 g/dl, 0.1-1 g/dl, 0.1-1.5 g/dl, 0.1-2 g/dl, 0.1-2501, 0.1-3 g/dl, 0.1-3.5 g/dl, 0.1-4 g/dl, 0.1-4.5 g/dl, 0.1-5 g/dl, 0.4-0.8 g/dl, 0.4-1 g/dl, 0.4-1.5 g/dl, 0.4-2 g/dl, 04-2.5 g/dl, 0.4-3 g/dl, 0.4-3.5 g/dl, 0.4-4 g/dl, 0.4-4.5 g/dl, 0.4-5 g/dl, 0.5-0.8 g/dl, 0.5-1 g/dl, 0.5-1.5 g/dl, 0.5-2 g/dl, 0.5-2.5 g/dl, 0.5-3 g/dl, 0.5-3.5 g/dl, 0.5-4 g/dl, 0.5-4.5 g/dl, 0.5-5 g/dl, 1-1.5 g/dl, 1-2 g/dl, 1-2.5 g/dl, 1-3 g/dl, 1-3.5 g/dl, 1-4 g/dl, 1-4.5 g/dl, 1-5 g/dl, 1.5-2 g/dl, 1.5-2.5 g/dl, 1.5-3 g/dl, 1.5-3.5 g/dl, 1.5-4 g/dl, 1.5-4.5 g/dl, 1.5-5 g/dl, 2-2.5 g/dl, 2-3 g/dl, 2-3.5 g/dl, 2-4 g/dl, 2-4.5 g/dl or 2-5 g/dl. In some embodiments, a subject treated for IDA in accordance with the methods described herein experiences mean increase in hemoglobin concentration of about 0.1 g/dl or more, about 0.2 g/dl or more, about 0.3 g/dl or more, about 0.4 g/dl or more, about 0.5 g/dl or more, about 1 g/dl or more, about 1.5 g/dl or more, about 2 g/dl or more, about 2.5 g/dl or more, about 3 g/dl or more, about 3.5 g/dl or more, about 4 g/dl or more, about 4.5 g/dl or more, or about 5 g/dl or more. In certain embodiments, the hemoglobin concentration does not increase by more than 2 g/dl, 3 g/dl, 4 g/dl or 5 g/dl. In some embodiments, a mean increase hemoglobin concentration results after the ferric citrate or a pharmaceutical composition thereof is administered to the subject for a certain period of time (e.g., 1 month, 2 months, 3 months, 4 months, 5 months, 6 months, 7 months, 8 months, 9 months, 10 months, 11 months, 12 months or more). In certain embodiments, a subject treated for IDA in accordance with the methods described herein experiences maintenance of their hemoglobin concentration such that their hemoglobin concentration remains substantially unchanged during administration of the ferric citrate or a pharmaceutical composition.

In specific aspects, provided herein are methods for increasing or maintaining hematocrit level in a subject with and/or diagnosed with IDA, comprising orally administering ferric citrate or a pharmaceutical composition thereof to the subject. See, e.g., Sections 4.2, in a, regarding the patient population treated, Section 4.3, infra, regarding the dosing and administration of ferric citrate or a pharmaceutical composition thereof, and Section 4.5, infra, regarding forms of ferric citrate and pharmaceutical compositions thereof. In specific embodiments, the subject is administered a low dose of ferric citrate at a certain frequency (e.g., every day, every other day, every 2 days, every 3 days, every 4 days, or every 5 days). In certain embodiments, the hematocrit level of the subject is assessed prior to administration of ferric citrate or a pharmaceutical composition thereof to the subject. In some embodiments, the hematocrit level of the subject is monitored after the administration of ferric citrate or a pharmaceutical composition thereof to the subject (e.g., monitored every month, 2 months, 3 months, 4 months, 5 months, 6 months or more). In certain embodiments, one or more other iron storage parameters, such as hemoglobin concentration, TSAT value, serum ferritin level, serum iron level, tissue iron level (e.g., stainable tissue iron level), TIBC value, plasma erythropoietin level, and/or FEP level, of the subject are assessed prior to administration of ferric citrate or a pharmaceutical composition thereof to the subject. In some embodiments, one or more other iron storage parameters, such as hemoglobin concentration, TSAT value, serum ferritin level, serum iron level, tissue iron level (e.g., stainable tissue iron level), TIBC value, plasma erythropoietin level, and/or FEP level, of the subject are monitored after the administration of ferric citrate or a pharmaceutical composition thereof to the subject (e.g., the one or more iron storage parameters are monitored every month, 2 months, 3 months, 4 months, 5 months, 6 months or more). In certain embodiments, the subject administered the ferric citrate or pharmaceutical composition thereof does not have and/or has not been diagnosed with chronic kidney disease and/or hyperphosphatemia.

The hematocrit, also referred to as packed cell volume or erythrocyte volume fraction, is the volume percentage of red blood cells in the blood. For non-IDA patients, the hematocrit is typically about 45% of blood volume for men and about 40% of blood volume for women. In IDA patients, however, the hematocrit is often significantly depleted due to poor iron absorption and/or poor iron storage capacity.

In certain embodiments, a subject treated for IDA in accordance with the methods described herein experiences an increase in hematocrit level of about 1-25%, 1-20%, 1-15%, 1-10%, 5-15%, 5-20%, 5-25%, 10-15%, 10-20%, 10-25%, 15-20%, 15-25%, or 20-25%. In some embodiments, a subject treated for IDA in accordance with the methods described herein experiences an increase in hematocrit level of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25% or more. In certain embodiments, an increase of hematocrit level results after the ferric citrate or a pharmaceutical composition thereof is administered to the subject for a certain period of time (e.g., 1 month, 2 months, 3 months, 4 months, 5 months, 6 months, 7 months, 8 months, 9 months, 10 months, 11 months, 12 months or more). In some embodiments, a subject treated for IDA in accordance with the methods described herein experiences maintenance of their hematocrit level such that their hematocrit remains substantially unchanged during administration of the ferric citrate or a pharmaceutical composition.

In specific aspects, provided herein are methods for decreasing or maintaining total iron-binding capacity (TIBC) value in a subject with and/or diagnosed with IDA, comprising orally administering ferric citrate or a pharmaceutical composition thereof to the subject. See, e.g., Sections 4.2, infra, regarding the patient population treated, Section 4.3, regarding the dosing and administration of ferric citrate or a pharmaceutical composition thereof, and Section 4.5, infra, regarding forms of ferric citrate and pharmaceutical compositions thereof. In specific embodiments, the subject is administered a low dose of ferric citrate at a certain frequency (e.g., every day, every other day, every 2 days, every 3 days, every 4 days, or every 5 days). In certain embodiments, the TIBC value of the subject is assessed prior to administration of ferric citrate or a pharmaceutical composition thereof to the subject. In some embodiments, the TIBC value of the subject is monitored after the administration of ferric citrate or a pharmaceutical composition thereof to the subject (e.g., monitored every month, 2 months, 3 months, 4 months, 5 months, 6 months or more). In certain embodiments, one or more other iron storage parameters, such as hemoglobin concentration, TSAT value, serum ferritin level, serum iron level, tissue iron level (e.g., stainable tissue iron level), hematocrit level, plasma erythropoietin level, and/or FEP level, of the subject are assessed prior to administration of ferric citrate or a pharmaceutical composition thereof to the subject. In some embodiments, one or more other iron storage parameters, such as hemoglobin concentration, TSAT value, serum ferritin level, serum iron level, tissue iron level (e.g., stainable tissue iron level), hematocrit level, plasma erythropoietin level, and/or FEP level, of the subject are monitored after the administration of ferric citrate or a pharmaceutical composition thereof to the subject (e.g., the one or more iron storage parameters are monitored every month, 2 months, 3 months, 4 months, 5 months, 6 months or more). In certain embodiments, the subject administered the ferric citrate or pharmaceutical composition thereof does not have and/or has not been diagnosed with chronic kidney disease and/or hyperphosphatemia.

Total iron-binding capacity (TIBC) is a measure of the blood's capacity to bind iron with the protein transferrin. TIBC is typically measured by drawing a blood sample and measuring the maximum amount of iron that the sample can carry. Thus, TIBC indirectly measures transferrin, which is a protein that transports iron in the blood. For non-IDA patients, a typical mass or molar measure of TIBC is in the range of 250-370 μg/dl or 45-66 μmol/L, respectively. In IDA patients, however, the TIBC is typically increased above these levels, as the body must produce more transferrin in an attempt to deliver iron to erythrocyte precursor cells to produce hemoglobin.

In certain embodiments, a subject treated for IDA in accordance with the methods described herein experiences a decrease in TIBC value of about 1-25%, 1-20%, 1-15%, 1-10%, 5-15%, 5-20%, 5-25%, 10-15%, 10-20%, 10-25%, 15-20%, 15-25%, or 20-25%. In some embodiments, a subject treated for IDA in accordance with the methods described herein experiences a decrease in TIBC value of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25% or more. In certain embodiments, a decrease of TIBC, value results after the ferric citrate or a pharmaceutical composition thereof is administered to the subject for a certain period of time (e.g., 1 month, 2 months, 3 months, 4 months, 5 months, 6 months, 7 months, 8 months, 9 months, 10 months, 11 months, 12 months or more). In some embodiments, a subject treated for IDA in accordance with the methods described herein experiences maintenance of their TIBC value such that their TIBC value remains substantially unchanged during administration of the ferric citrate or a pharmaceutical composition.

In specific aspects, provided herein are methods for increasing or maintaining serum iron level in a subject with and/or diagnosed with IDA, comprising orally administering ferric citrate or a pharmaceutical composition thereof to the subject. See, e.g., Sections 4.2, infra, regarding the patient population treated, Section 4.3, infra, regarding the dosing and administration of ferric citrate or a pharmaceutical composition thereof, and Section 4.5, infra, regarding forms of ferric citrate and pharmaceutical compositions thereof. In specific embodiments, the subject is administered a low dose of ferric citrate at a certain frequency (e.g., every day, every other day, every 2 days, every 3 days, every 4 days, or every 5 days). In certain embodiments, the serum iron level is assessed prior to administration of ferric citrate or a pharmaceutical composition thereof to the subject. In some embodiments, the serum iron level of the subject is monitored after the administration of ferric citrate or a pharmaceutical composition thereof to the subject (e.g., monitored every month, 2 months, 3 months, 4 months, 5 months, 6 months or more). In certain embodiments, one or more other iron storage parameters, such as hemoglobin concentration, TSAT value, serum ferritin level, tissue iron level (e.g., stainable tissue iron level), hematocrit level, TIBC value, plasma erythropoietin level, and/or PEP level, of the subject are assessed prior to administration of ferric citrate or a pharmaceutical composition thereof to the subject. In some embodiments, one or more other iron storage parameters, such as hemoglobin concentration, TSAT value, serum ferritin level, tissue iron level (e.g., stainable tissue iron level), hematocrit level, TIBC value, plasma erythropoietin level, and/or FEP level, of the subject are monitored after the administration of ferric citrate or a pharmaceutical composition thereof to the subject (e.g., the one or more iron storage parameters are monitored every month, 2 months, 3 months, 4 months, 5 months, 6 months or more). In certain embodiments, the subject administered the ferric citrate or pharmaceutical composition thereof does not have and/or has not been diagnosed with chronic kidney disease and/or hyperphosphatemia.

The serum pool of iron is the fraction of all iron in the body that circulates in the blood and bound primarily to transferrin. The iron in this pool turns over very quickly and represents iron in transit from one location to another. Serum iron level is a measure of the amount of this pool of circulating iron in the blood. A normal serum iron level is usually 65-176 μg/dl for men, 50-170 μg/dl for women, and 50-120 μg/dl for children. In an IDA patient, however, the serum iron level is typically reduced below the normal range as the body loses its ability to absorb and/or store iron.

In certain embodiments, a subject treated for IDA in accordance with the methods described herein experiences mean increase in serum iron level of about 1-100%, 1-95%, 10-95%, 10-90%, 10-85%, 10-80%, 10-75%, 10-70%, 10-65%, 10-60%, 10-50%, 10-45%, 10-40%, 10-35%, 10-30%, 10-25%, 10-20%, 20-30%, 20-40%, 20-50%, 20-60%, 20-70%, 20-80%, 20-90%, 30-90%, 30-80%, 30-70%, 30-60%, 30-50%, 30-40%, 40-90%, 40-80%, 40-70%, 40-60%, 40-50%, 50-90%, 50-80%, 50-70%, 50-65%, 50-60%, 60-90%, 60-80%, 60-75%, 60-70%, 70-90%, 70%-80%, or 80-90%. In some embodiments, a subject treated for IDA in accordance with the methods described herein experiences mean increase in serum iron level of 10%, 15%, 20%, 25%, 30%, 35%, 40%, 45%, 50%, 55%, 60%, 65%, 70%, 75%, 80%, 85%, 90%, 95% or more. In certain embodiments, a mean increase of serum iron level results after the ferric citrate or a pharmaceutical composition thereof is administered to the subject for a certain period of time (e.g., 1 month, 2 months, 3 months, 4 months, 5 months, 6 months, 7 months, 8 months, 9 months, 10 months, 11 months, 12 months or more). In some embodiments, a subject treated for IDA in accordance with the methods described herein experiences maintenance of their serum iron level such that their serum iron level remains substantially unchanged during administration of the ferric citrate or a pharmaceutical composition.

In specific aspects, provided herein are methods for decreasing or maintaining plasma erythropoietin level in a subject with and/or diagnosed with IDA, comprising orally administering ferric citrate or a pharmaceutical composition thereof to the subject. See, e.g., Sections 4.2, infra, regarding the patient population treated, Section 4.3, infra, regarding the dosing and administration of ferric citrate or a pharmaceutical composition thereof, and Section 4.5, infra, regarding forms of ferric citrate and pharmaceutical compositions thereof. In specific embodiments, the subject is administered a low dose of ferric citrate at a certain frequency (e.g., every day, every other day, every 2 days, every 3 days, every 4 days, or every 5 days). In certain embodiments, the plasma erythropoietin level of the subject is assessed prior to administration of ferric citrate or a pharmaceutical composition thereof to the subject. In some embodiments, the plasma erythropoietin level of the subject is monitored after the administration of ferric citrate or a pharmaceutical composition thereof to the subject (e.g., monitored every month, 2 months, 3 months, 4 months, 5 months, 6 months or more). In certain embodiments, one or more other iron storage parameters, such as hemoglobin concentration, TSAT value, serum ferritin level, serum iron level, tissue iron level (e.g., stainable tissue iron level), hematocrit level, TIBC value, and/or FEP level, of the subject are assessed prior to administration of ferric citrate or a pharmaceutical composition thereof to the subject. In some embodiments, one or more other iron storage parameters, such as hemoglobin concentration, TSAT value, serum ferritin level, serum iron level, tissue iron level (e.g., stainable tissue iron level), hematocrit level, TIBC value, and/or FEP level, of the subject are monitored after the administration of ferric citrate or a pharmaceutical composition thereof to the subject (e.g., the one or more iron storage parameters are monitored every month, 2 months, 3 months, 4 months, 5 months, 6 months or more). In certain embodiments, the subject administered the ferric citrate or pharmaceutical composition thereof does not have and/or has not been diagnosed with chronic kidney disease and/or hyperphosphatemia.

Erythropoietin is a renal glycoprotein hormone that is an obligatory growth factor for the proliferation and differentiation of committed erythroid progenitor cells. Plasma erythropoietin level usually increases as the hematocrit level decreases. A normal plasma erythropoietin level is usually 4.1-19.5 mU/ml for adults, and 9-28 mU/ml for children. In an IDA patient, however, the plasma erythropoietin level is typically increased above the normal range as the body loses its ability to absorb and/or store iron.

In certain embodiments, a subject treated for IDA in accordance with the methods described herein experiences mean decrease in plasma erythropoietin level of about 1-100%, 1-95%, 10-95%, 10-90%, 10-85%, 10-80%, 10-75%, 10-70%, 10-65%, 10-60%, 10-50%, 10-45%, 10-40%, 10-35%, 10-30%, 10-25%, 10-20%, 20-30%, 20-40%, 20-50%, 20-60%, 20-70%, 20-80%, 20-90%, 30-90%, 30-80%, 30-70%, 30-60%, 30-50%, 30-40%, 40-90%, 40-80%, 40-70%, 40-60%, 40-50%, 50-90%, 50-80%, 50-70%, 50-65%, 50-60%, 60-90%, 60-80%, 60-75%, 60-70%, 70-90%, 70%-80%, or 80-90%. In some embodiments, a subject treated for IDA in accordance with the methods described herein experiences mean decrease in plasma erythropoietin level of 10%, 15%, 20%, 25%, 30%, 35%, 40%, 45%, 50%, 55%, 60%, 65%, 70%, 75%, 80%, 85%, 90%, 95% or more. In certain embodiments, a mean increase of plasma erythropoietin level results after the ferric citrate or a pharmaceutical composition thereof is administered to the subject for a certain period of time (e.g., 1 month, 2 months, 3 months, 4 months, 5 months, 6 months, 7 months, 8 months, 9 months, 10 months, 11 months, 12 months or more). In some embodiments, a subject treated for IDA in accordance with the methods described herein experiences maintenance of their plasma erythropoietin level such that their plasma erythropoietin level remains substantially unchanged during administration of the ferric citrate or a pharmaceutical composition.

In specific aspects, provided herein are methods for decreasing or maintaining free erythrocyte protoporphyrin (FEP) level in a subject with and/or diagnosed with IDA, comprising orally administering ferric citrate or a pharmaceutical composition thereof to the subject. See, e.g., Sections 4.2, infra, regarding the patient population treated, Section 4.3, infra, regarding the dosing and administration of ferric citrate or a pharmaceutical composition thereof, and Section 4.5, infra, regarding forms of ferric citrate and pharmaceutical compositions thereof. In specific embodiments, the subject is administered a low dose of ferric citrate at a certain frequency (e.g., every day, every other day, every 2 days, every 3 days, every 4 days, or every 5 days). In certain embodiments, the FEP level of the subject is assessed prior to administration of ferric citrate or a pharmaceutical composition thereof to the subject. In some embodiments, the FEP level of the subject is monitored after the administration of ferric citrate or a pharmaceutical composition thereof to the subject (e.g., monitored every month, 2 months, 3 months, 4 months, months, 6 months or more). In certain embodiments, one or more other iron storage parameters, such as hemoglobin concentration, TSAT value, serum ferritin level, serum iron level, tissue iron level (e.g., stainable tissue iron level), hematocrit level, TIBC value, and/or plasma erythropoietin level, of the subject are assessed prior to administration of ferric citrate or a pharmaceutical composition thereof to the subject. In some embodiments, one or more other iron storage parameters, such as hemoglobin concentration, TSAT value, serum ferritin level, serum iron level, tissue iron level (e.g., stainable tissue iron level), hematocrit level, TIBC value, and/or plasma erythropoietin level, of the subject are monitored after the administration of ferric citrate or a pharmaceutical composition thereof to the subject (e.g., the one or more iron storage parameters are monitored every month, 2 months, 3 months, 4 months, 5 months, 6 months or more) In certain embodiments, the subject administered the ferric citrate or pharmaceutical composition thereof does not have and/or has not been diagnosed with chronic kidney disease and/or hyperphosphatemia.

When there is a lack of iron in the bone marrow for incorporation into the heme group during hemoglobin synthesis, zinc is incorporated instead and forms a compound called zinc protoporphyrin (ZPP). Free erythrocyte protoporphyrin (FEP) is the compound left over after the zinc ion has been removed during the extraction and chemical measurement process. A rise in the FEP level is one of the first indicators of insufficient iron in the bone marrow. A normal FEP level is usually: 10-40 μg/dl red blood cells. In an IDA patient, however, the serum iron level is typically increased above the normal range as the body loses its ability to absorb and/or store iron.

In certain embodiments, a subject treated for IDA in accordance with the methods described herein experiences mean decrease in FEP level of about 1-100%, 1-95%, 10-95%, 10-90%, 10-85%, 10-80%, 10-75%, 10-70%, 10-65%, 10-60%, 10-50%, 10-45%, 10-40%, 10-35%, 10-30%, 10-25%, 10-20%, 20-30%, 20-40%, 20-50%, 20-60%, 20-70%, 20-80%, 20-90%, 30-90%, 30-80%, 30-70%, 30-60%, 30-50%, 30-40%, 40-90%, 40-80%, 40-70%, 40-60%, 40-50%, 50-90%, 50-80%, 50-70%, 50-65%, 50-60%, 60-90%, 60-80%, 60-75%, 60-70%, 70-90%, 70% 80%, or 80-90%. In some embodiments, a subject treated for IDA in accordance with the methods described herein experiences mean decrease in FEP level of 10%, 15%, 20%, 25%, 30%, 35%, 40%, 45%, 50%, 55%, 60%, 65%, 70%, 75%, 80%, 85%, 90%, 95% or more. In certain embodiments, a mean increase of FEP level results after the ferric citrate or a pharmaceutical composition thereof is administered to the subject for a certain period of time (e.g., 1 month, 2 months, 3 months, 4 months, 5 months, 6 months, 7 months, 8 months, 9 months, 10 months, 11 months, 12 months or more). In some embodiments, a subject treated for IDA in accordance with the methods described herein experiences maintenance of their FEP level such that their FEP level remains substantially unchanged during administration of the ferric citrate or a pharmaceutical composition.

There are typically three means by which IDA can be treated. The first approach is by eating foods that are high in iron. If that is insufficient, then a clinician may prescribe oral or intravenous (IV) iron supplements. Intravenous (IV) iron supplementation is a method of delivering iron by injection with a needle, either through a muscle or into a vein. IDA patients who are receiving IV iron usually do so because they cannot tolerate oral iron. Intravenous iron is delivered into the IDA patient's vein through a needle that is attached to an IV bag that contains an iron solution. The procedure takes place in a doctor's office or a clinic and may take up to several hours, depending on which treatment the physician has prescribed. The patient usually receives iron injections over the course of several visits until his or her iron levels are correct. In some instances, an IDA patient may require permanent IV iron supplementation. IV iron is associated with short-term side effects such as gastrointestinal pains (e.g., nausea and cramps), breathing problems, skin problems (e.g., rash), chest pain, low blood pressure, anaphylaxis, and death, as well as long-term toxicity, including the development of atherosclerosis, infection, and increased mortality (Quinibi, Arzneimittelforschung (2010) 60, 399-412). Further, many clinics, particularly community sites, are ill-equipped to administer intravenous iron. This has left a majority of IDA patients without intravenous iron treatment.

In addition, IDA patients may also take one or more erythropoiesis-stimulating agents (ESAs) in an effort to control anemia. ESAs work by helping the body to produce red blood cells. These red blood cells are then released from the bone marrow into the bloodstream where they help maintain blood iron levels. Erythropoiesis-stimulating agents, commonly abbreviated as ESAs, are agents that are similar in structure and/or function to the cytokine erythropoietin, which stimulates red blood cell production (erythropoeisis) in the body. Typical ESAs, structurally and biologically, are similar to naturally occurring protein erythropoietin. Examples of commercially available ESAs include Erythropoictin (Epo), Epoetin alfa (Procrit/Epogen), Epoetin beta (NeoRecormon), Darbepoetin alfa (Aranesp), and Methoxy polyethylene glycol-epoetin beta (Mircera). The two ESAs presently approved for marketing in the U.S. are Epoetin alfa (Procrit, Epogen), and Darbepoietin alfa (Aranesp).

The side effects that occur most often with ESA use include: high blood pressure; swelling; fever; dizziness; nausea; and pain at the site of the injection, among others. In addition to these side effects, there are several safety issues that result from ESA use. ESAs increase the risk of venous thromboembolism (blood clots in the veins). ESAs can also cause hemoglobin to rise too high, which puts the patient at higher risk for heart attack, stroke, heart failure, and death. In addition, ESAs may in certain cases worsen iron depletion and lead to an increase in thrombocytosis.

In specific aspects, provided herein are methods for decreasing or maintaining the intravenous iron and/or erythropoeisis-stimulating agent(s) intake by a subject with and/or diagnosed with IDA, comprising orally administering ferric citrate or a pharmaceutical composition thereof to the subject. See, e.g., Sections 4.2, infra, regarding the patient population treated, Section 4.3, infra, regarding the dosing and administration of ferric citrate or a pharmaceutical composition thereof, and Section 4.5, Infra, regarding forms of ferric citrate and pharmaceutical compositions thereof. In specific embodiments, the subject is administered a low dose of ferric citrate at a certain frequency (e.g., every day, every other day, every 2 days, every 3 days, every 4 days, or every 5 days). In certain embodiments, one or more iron storage parameters, such as hemoglobin concentration, TSAT value, serum ferritin level, serum iron level, tissue iron level (e.g., stainable tissue iron level), hematocrit level, TIBC value, plasma erythropoietin level, and/or FIE level, of the subject are assessed prior to administration of ferric citrate or a pharmaceutical composition thereof to the subject. In some embodiments, one or more iron storage parameters, such as hemoglobin concentration, TSAT value, serum ferritin level, serum iron level, tissue iron level (e.g., stainable tissue iron level), hematocrit level, TIBC value, plasma erythropoietin level, and/or FEP level, of the subject are monitored after the administration of ferric citrate or a pharmaceutical composition thereof to the subject (e.g., every month, 2 months, 3 months, 4 months, 5 months, 6 months or more). In certain embodiments, the subject administered the ferric citrate or pharmaceutical composition thereof does not have and/or has not been diagnosed with chronic kidney disease and/or hyperphosphatemia.

In certain embodiments, a subject treated for IDA in accordance with the methods described herein experiences a mean reduction in average cumulative IV iron intake of about 1-25%, 1-20%, 1-15%, 1-10%, 5-15%, 5-20%, 5-25%, 10-15%, 10-20%, 10-25%, 15-20%, 15-25%, 20-25%, 1-100%, 20-25%, 20-30%, 20-40%, 20-50%, 20-60%, 20-70%, 20-80%, 20-90%, 25-30%, 25-45%, 25-50%, 25-75%, 25-80%, 25-85%, 25-90%, 25-95%, 30-40%, 30-60%, 30-70%, 30-80%, 30-90%, 40-50%, 40-80%, 40-95%, 50-60%, 50-75%, 50-95%, 60-70%, 60-90%, 60-95%, 75-85%, 75-95%, or 75-100%. In some embodiments, a subject treated for IDA in accordance with the methods described herein a mean reduction in average cumulative IV iron intake of 1%, 2%, 3%, 4%, 5%, 6%, 7% 8%, 9%, 10%, 11%, 12%, 13%, 14%, 15%, 16%, 17%, 18%, 19%, 20%, 21%, 22%, 23%, 24%, 25%, 30%, 35%, 40%, 50%, 55%, 60%, 65%, 70%, 75%, 80%, 85%, 909%, 95% or more. In certain embodiments, a mean reduction in average cumulative IV iron intake results after the ferric citrate or a pharmaceutical composition thereof is administered to the subject for a certain period of time (e.g., 1 month, 2 months, 3 months, 4 months, 5 months, 6 months, 7 months, 8 months, 9 months, 10 months, 11 months, 12 months or more).

In certain embodiments, a subject treated for IDA in accordance with the methods described herein experiences a decrease in median ESA intake of about 1-25%, 1-20%, 1-15%, 1-10%, 5-15%, 5-20%, 5-25%, 10-15%, 10-20%, 10-25%, 15-20%, 15-25%, 20-25%, 1-100%, 20-25%, 20-30%, 20-40%, 20-50%, 20-60%, 20-70%, 20-80%, 20-90%, 25-30%, 25-45%, 25-50%, 25-75%, 25-80%, 25-85%, 25-90%, 25-95%, 30-40%, 30-60%, 30-70%, 30-80%, 30-90%, 40-50%, 40-80%, 40-95%, 50-60%, 50-75%, 50-95%, 60-70%, 60-90%, 60-95%, 75-85%, 75-95%, or 75-100%. In some embodiments, a subject treated for IDA in accordance with the methods described herein a decrease in median ESA intake of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 50%, 55%, 60%, 65%, 70%, 75%, 80%, 85%, 90%, 95% or more. In certain embodiments, a decrease in median ESA intake results after the ferric citrate or a pharmaceutical composition thereof is administered to the subject for a certain period of time (e.g., 1 month, 2 months, 3 months, 4 months, 5 months, 6 months, 7 months, 8 months, 9 months, 10 months, 11 months, 12 months or more).

### 4.2. Patient Population

The terms “patient” and “subject” are used herein interchangeably to refer to an animal. In certain embodiments, a patient treated in accordance with the methods disclosed herein is a mammal, such as a non-primate (e.g., a cow, pig, horse, cat, dog, rat, etc.) or a primate (e.g., a monkey or human). In a preferred embodiment, a patient treated in accordance with the methods disclosed herein is a human.

In certain embodiments, a patient treated in accordance with the methods disclosed herein is male or non-pregnant, non-breastfeeding female. In some embodiments, a patient treated in accordance with the methods disclosed herein is a human 18 years of age or older.

In certain embodiments, a patient treated in accordance with the methods disclosed herein does not have and/or has not been diagnosed with hyperphosphatemia. In other embodiments, a patient treated in accordance with the methods disclosed herein is hyperphosphatemia.

In some embodiments, a patient treated in accordance with the methods disclosed herein has and/or has been diagnosed as having IDA associated with chronic kidney disease (CKD). CKD is a condition characterized by a gradual loss of kidney function over time, and IDA is a common complication of CKD. All individuals with a glomerular filtration rate (CFR) <60 ml/min/1.73 m2 for 3 months are classified as having CKD, irrespective of the presence or absence of kidney damage. Based on the severity, CKD can be classified in five stages. Stage 1 is the mildest and usually causing few symptoms. Stage 2 is characterized by mild reduction in GFR (60-89 ml/min/1.73 m²) with kidney damage. Stage 3 is characterized by moderate reduction in GFR (30.59 ml/min/1.73 m²). Stage 4 is characterized by severe reduction in GFR (15-29 ml/min/1.73 m²). Stage 5 is characterized by established kidney failure (GFR <15 ml/min/1.73 m²). Stage 5 is a severe illness with poor life expectancy if untreated. Those individuals with CKD who require either dialysis or kidney transplantation are typically referred to as end-stage renal disease (ESRD) patients. Therefore, a patient is traditionally classified as an ESRD patient when he or she reaches the conclusion of the non-dialysis dependent, earlier stages, of CKD. Prior to then, those patients are referred to as non-dialysis dependent CKD (ND-CKD) patients. Typically, patients progress through stages 1 through 4 before dialysis is medically necessary. However, patients at stage 5 who have not yet started dialysis or who have not been recommended for transplantation are also non-dialysis dependent CKD patients. In various embodiments, the IDA patients are stage 3-5 CKD patients.

In some embodiments, a patient treated in accordance with the methods disclosed herein does not have and/or has not been diagnosed with chronic kidney disease. In certain embodiments, a patient treated in accordance with the methods disclosed herein does not have and/or has not been diagnosed with stage 1, 2, 3, 4, or 5 chronic kidney disease. In some embodiments, a patient treated in accordance with the methods disclosed herein does not have and/or has not been diagnosed with end-stage chronic kidney disease. In certain embodiments, a patient treated in accordance with the methods disclosed herein does not have and/or has not been diagnosed with chronic kidney disease and/or hyperphosphatemia.

In certain other embodiments, a patient treated in accordance with the methods disclosed herein has and/or has been diagnosed with chronic kidney disease. In some embodiments, a patient treated in accordance with the methods disclosed herein has and/or has been diagnosed with stage 1, 2, 3, 4, or 5 chronic kidney disease. In certain embodiments, a patient treated in accordance with the methods disclosed herein has and/or has been diagnosed with end-stage chronic kidney disease. In some embodiments, a patient treated in accordance with the methods disclosed herein has and/or has been diagnosed with chronic kidney disease and is receiving dialysis. In other embodiments, a patient treated in accordance with the methods disclosed herein has and/or has been diagnosed with chronic kidney disease and is not receiving dialysis.

In certain embodiments, a patient treated in accordance with the methods disclosed herein has a hemoglobin concentration of approximately 9 grams/dl or greater, such as approximately 9.5 grams/dl, 10 grams/dl, 11 grams/dl, 11.5 grams/dl, or 12 grams/dl, prior to administration of ferric citrate or a pharmaceutical composition thereof. In some embodiments, a patient treated in accordance with the methods disclosed herein has a hemoglobin concentration of approximately 9 grams/dl and less than or equal to approximately 12.5 grams/dl, 12 grams/dl or 11.5 grams/dl prior to administration of ferric citrate or a pharmaceutical composition thereof. In certain embodiments, a patient treated in accordance with the methods disclosed herein has a hemoglobin concentration of approximately 6 grams/dl to approximately 8 grams/dl, approximately 6 grams/dl to approximately 10 grams/dl, approximately 6 grams/dl to approximately 12 grams/dl, approximately 7 grams/dl to approximately 9 grams/dl, approximately 7 grams/dl to approximately 11 grams/dl, approximately 7 grams/dl to approximately 13 grams/dl, approximately 8 grams/dl to approximately 10 grams/dl, approximately 8 grams/dl to approximately 12 grams/dl, approximately 9 grams/dl to approximately 11 grams/dl, approximately 9 grams/dl to approximately 12 grams/dl, approximately 9 grams/dl to approximately 13 grams/dl, approximately 10 grams/dl to approximately 11 grams/dl, approximately 10 grams/dl to approximately 12 grams/dl, approximately 10 grams/dl to approximately 13 gams/dl, approximately 11 grams/dl to approximately 12 grams/dl, approximately 11 grams/dl to approximately 13 grams/dl, or approximately 12 grams/dl to approximately 13 grams/dl prior to administration of ferric citrate or a pharmaceutical composition thereof.

In certain embodiments, a patient treated in accordance with the methods disclosed herein has a TSAT value of less than 50%, 45%, 40%, 35%, 30%, 25%, 20%, 15% 12%, or 10% prior to administration of ferric citrate or a pharmaceutical composition thereof in some embodiments, a patient treated in accordance with the methods disclosed herein has a TSAT value of 5% to 50%, 5% to 45%, 5% to 40%, 5% to 35%, 5% to 30%, 5% to 25%, 5% to 20%, 5% to 15%, 5% to 12%, 5% to 10%, 10% to 50%, 10% to 45%, 10% to 40%, 10% to 35%, 10% to 30%, 10% to 25%, 10% to 20%, 10% to 15%, 10% to 12%, 12% to 50%, 12% to 45%, 12% to 40%, 12% to 35%, 12% to 30%, 12% to 25%, 12% to 20%, 12% to 15%, 15% to 50%, 15% to 45%, 15% to 40%, 15% to 35%, 15% to 30%, 15% to 25%, 15% to 20%, 20% to 50%, 20% to 45%, 20% to 40%, 20% to 35%, 20% to 30%, 20% to 25%, 30% to 50%, 30% to 45%, 30% to 40%, 30% to 35%, 40% to 50%, 40% to 45%, or 45% to 50% prior to administration of ferric citrate or a pharmaceutical composition thereof. In certain embodiments wherein the patient treated in accordance with the methods disclosed herein is a female, the patient has a TSAT value of 5% to 45%, 5% to 35%, 5% to 25%, 5% to 15%, 5% to 12%, 5% to 10%, 10% to 45%, 10% to 35%, 10% to 25%, 10% to 15%, 10% to 12%, 12% to 45%, 12% to 35%, 12% to 25%, 12% to 15%, 20% to 45%, 20% to 35%, 20% to 25%, 30% to 45%, 30% to 35%, or 40% to 45% prior to administration of ferric citrate or a pharmaceutical composition thereof. In certain embodiments wherein the patient treated in accordance with the methods disclosed herein is a male, the patient has a TSAT value of 5% to 50%, 5% to 40%, 5% to 30%, 5% to 20%, 5% to 15%, 5% to 10%, 10% to 50%, 10% to 40%, 10% to 30%, 10% to 20%, 10% to 15%, 15% to 50%, 15% to 40%, 15% to 30%, 15% to 25%, 15% to 20%, 20% to 50%, 20% to 40%, 20% to 30%, 20% to 25%, 30% to 50%, 30% to 40%, 30% to 35%, 40% to 50%, 40% to 45%, or 45% to 50% prior to administration of ferric citrate or a pharmaceutical composition thereof.

In certain embodiments, a patient treated in accordance with the methods disclosed herein has a serum ferritin level of less than 300 ng/ml (e.g., less than or equal to 275 ng/ml, less than or equal to 250 ng/ml, less than or equal to 225 ng/ml, less than or equal to 200 ng/ml, less than or equal to 175 ng/ml, less than or equal to 150 ng/ml, less than or equal to 125 ng/ml, less than or equal to 100 ng/ml, less than or equal to 75 ng/ml, less than or equal to 50 ng/ml, less than or equal to 25 ng/ml, less than or equal to 15 ng/ml, less than or equal to 10 ng/ml, or less than or equal to 5 ng/ml) prior to administration of ferric citrate or a pharmaceutical composition thereof. In some embodiments, a patient treated in accordance with the methods disclosed herein has a serum ferritin level of approximately 5 ng/ml, 10 ng/ml, 15 ng/ml, 20 ng/ml, 25 ng/ml, 30 ng/ml, 35 ng/ml, 40 ng/ml, 45 ng/ml, 50 ng/ml, 55 ng/ml, 60 ng/ml, 65 ng/ml, 70 ng/ml, 75 ng/ml, 80 ng/ml, 85 ng/ml, 90 ng/ml, 95 ng/ml, 100 ng/ml, 125 ng/ml, 150 ng/ml, 175 ng/ml, 200 ng/ml, 225 ng/ml, 250 ng/ml, 275 ng/ml, or 300 ng/ml prior to administration of ferric citrate or a pharmaceutical composition thereof. In certain embodiments, a patient treated in accordance with the methods disclosed herein has a serum ferritin level of approximately 5 ng/ml to approximately 15 ng/ml, approximately 5 ng/ml to approximately 25 ng/ml, approximately 5 ng/ml to approximately 50 ng/ml, approximately 15 ng/ml to approximately 25 ng/ml, approximately 15 ng/ml to approximately 50 ng/ml, approximately 15 ng/ml to approximately 75 ng/ml, approximately 25 ng/ml to approximately 50 ng/ml, approximately 25 ng/ml to approximately 75 ng/ml, approximately 25 ng/ml to approximately 100 ng/ml, approximately 50 ng/ml to approximately 75 ng/ml, approximately 50 ng/ml to approximately 100 ng/ml, approximately 50 ng/ml to approximately 150 ng/ml, approximately 75 ng/ml to approximately 100 ng/ml, approximately 75 ng/ml to approximately 150 ng/ml, approximately 100 ng/ml to approximately 150 ng/ml, approximately 150 ng/ml to approximately 200 ng/ml, approximately 150 ng/ml to approximately 250 ng/ml, approximately 100 ng/ml to approximately 300 ng/ml, approximately 200 ng/ml to approximately 300 ng/ml, or approximately 250 ng/ml to approximately 300 ng/ml prior to administration of ferric citrate or a pharmaceutical composition thereof. In certain embodiments, a patient treated in accordance with the methods disclosed herein has a serum ferritin level of between 5 ng/ml to 300 ng/ml (e.g., between 5 ng/ml to 250 ng/ml, between 5 ng/ml to 150 ng/ml, between 5 ng/ml to 100 ng/ml, between 5 ng/ml to 75 ng/ml, between 5 ng/ml to 50 ng/ml, between 5 ng/ml to 25 ng/ml, between 5 ng/ml to 15 ng/ml, or between 5 ng/ml to 10 ng/ml) prior to administration of ferric citrate or a pharmaceutical composition thereof.

In certain embodiments, a patient treated in accordance with the methods disclosed herein has a hematocrit level of less than 45%, 404% 35%, 30%, 25%, 204% 15% or 10% prior to administration of ferric citrate or a pharmaceutical composition thereof. In some embodiments, a patient treated in accordance with the methods disclosed herein has a hematocrit level of 10% to 15%, 10% to 20%, 10% to 25%, 10% to 30%, 10% to 35%, 10% to 40%, 10% to 45%, 15% to 20%, 15% to 25%, 15% to 30%, 15% to 35%, 15% to 40%, 15% to 45%, 20% to 25%, 20% to 30%, 20% to 35%, 20% to 40%, 25% to 45%, 25% to 30%, 25% to 35%, 25% to 40%, 25% to 45%, 30/o to 35%, 30/o to 40%, 30/o to 45%, 35% to 40%, 35% to 45%, or 40% to 45%, prior to administration of ferric citrate or a pharmaceutical composition thereof.

In certain embodiments, a patient treated in accordance with the methods disclosed herein has a TIBC value of more than 390 μg/dl (e.g., more than or equal to 390 μg/dl, more than or equal to 400 μg/dl, more than or equal to 450 μg/dl, more than or equal to 450 μg/dl, more than or equal to 500 μg/dl, more than or equal to 550 μg/dl, inure than or equal to 600 μg/dl, more than or equal to 650 μg/dl, more than or equal to 700 μg/dl, more than or equal to 800 μg/dl, more than or equal to 900 μr/dl, more than or equal to 1000 μg/dl, more than or equal to 1100 μg/dl, or more than or equal to 1200 μg/dl) prior to administration of ferric citrate or a pharmaceutical composition thereof. In some embodiments, a patient treated in accordance with the methods disclosed herein has a TIBC value of approximately 390 μg/dl, 400 μg/dl, 450 μg/dl, 500 μg/dl, 550 μg/dl, 600 μg/d, 650 μg/dl, 700 μg/dl, 800 μg/dl, 900 μg/dl, 1000 μg/dl, 1100 μg/dl, or 1200 μg/dl prior to administration of ferric citrate or a pharmaceutical composition thereof. In certain embodiments, a patient treated in accordance with the methods disclosed herein has a TIBC value of approximately 390 μg/dl to approximately 600 μg/dl, approximately 390 μg/dl to approximately 800 μg/dl, approximately 390 μg/dl to approximately 1000 μg/dl, approximately 390 μg/dl to approximately 1200 μg/dl, approximately 500 μg/dl to approximately 700 μg/dl, approximately 500 μg/dl to approximately 900 μg/dl, approximately 500 μg/dl to approximately 1100 μg/dl, approximately 600 μg/dl to approximately 800 μg/dl, approximately 600 μg/dl to approximately 1000 μg/dl, approximately 600 μg/dl to approximately 1200 μg/dl, approximately 700 μg/dl to approximately 900 μg/dl, approximately 700 μg/dl to approximately 1100 μg/dl, approximately 800 μg/dl to approximately 1000 μg/dl, approximately 800 μg/dl to approximately 1200 μg/dl, approximately 900 μg/dl to approximately 1100 Ng/dl, or approximately 1000 μg/dl to approximately 1200 μg/dl ml prior to administration of ferric citrate or a pharmaceutical composition thereof.

In certain embodiments, a patient treated in accordance with the methods disclosed herein has a tissue iron level (e.g., stainable tissue iron level) of grade 2 prior to administration of ferric citrate or a pharmaceutical composition thereof. In certain embodiments, a patient treated in accordance with the methods disclosed herein has a tissue iron level (e.g., stainable tissue iron level) of grade 1 prior to administration of ferric citrate or a pharmaceutical composition thereof. In certain embodiments, a patient treated in accordance with the methods disclosed herein has a tissue iron level (e.g., stainable tissue iron level) of grade 0 prior to administration of ferric citrate or a pharmaceutical composition thereof.

In certain embodiments, a patient treated in accordance with the methods disclosed herein has a serum iron level of less than 60 μg/dl (e.g., less than or equal to 50 μg/dl, less than or equal to 40 μg/dl, less than or equal to 30 μg/dl, less than or equal to 20 μg/dl, or less than or equal to 10 μg/dl) prior to administration of ferric citrate or a pharmaceutical composition thereof. In some embodiments, a patient treated in accordance with the methods disclosed herein has a serum iron level of approximately 5 μg/dl, 10 μg/dl, 15 μg/dl, 20 μg/dl, 25 μg/dl, 30 μg/l, 40 μg/dl, 50 μg/dl, or 60 μg/dl prior to administration of ferric citrate or a pharmaceutical composition thereof. In certain embodiments, a patient treated in accordance with the methods disclosed herein has a serum iron level of approximately 10 μg/dl to approximately 20 μg/dl, approximately 10 μg/dl to approximately 30 μg/dl, approximately 10 μg/dl to approximately 40 μg/dl, approximately 10 μg/dl to approximately 50 μg/dl, approximately 10 μg/dl to approximately 60 μg/dl, approximately 20 μg/dl to approximately 30 μg/dl, approximately 20 μg/dl to approximately 40 μg/dl, approximately 20 μg/dl to approximately 50 μg/dl, approximately 20 μg/dl to approximately 60 Well, approximately 30 μg/dl to approximately 40 μg/dl, approximately 30 μg/dl to approximately 50 μg/dl, approximately 30 μg/dl to approximately 60 μg/dl, approximately 40 μg/dl to approximately 50 μg/dl, or approximately 40 μg/dl to approximately 60 μg/dl prior to administration of ferric citrate or a pharmaceutical composition thereof.

In certain embodiments, a patient treated in accordance with the methods disclosed herein has a plasma erythropoietin level of more than 20 mU/ml (e.g., more than or equal to 20 mU/ml, more than or equal to 25 mU/ml, more than or equal to 30 mU/ml, more than or equal to 40 mU/ml, more than or equal to 50 mU/ml, or more than or equal to 60 mU/ml) prior to administration of ferric citrate or a pharmaceutical composition thereof in some embodiments, a patient treated in accordance with the methods disclosed herein has a plasma erythropoietin level of approximately 20 mU/ml, 25 mU/ml, 30 mU/ml, 35 mU/ml, 40 mU/ml, 45 mU/ml, 50 mU/ml, 55 mU/ml, or 60 mU/ml prior to administration of ferric citrate or a pharmaceutical composition thereof. In certain embodiments, a patient treated in accordance with the methods disclosed herein has a plasma erythropoietin level of approximately 20 mU/ml to approximately 30 mU/ml, approximately 20 mU/ml to approximately 40 mU/ml, approximately 20 mU/ml to approximately 50 mU/ml, approximately 20 mU/ml to approximately 60 mU/ml, approximately 30 mU/ml to approximately 40 mU/ml, approximately 30 mU/ml to approximately 50 mU/ml, approximately 30 mU/ml to approximately 60 mU/ml, approximately 40 mU/ml to approximately 50 mU/ml, approximately 40 mU/ml to approximately 60 mU/ml, or approximately 50 mU/ml to approximately 60 mU/ml prior to administration of ferric citrate or a pharmaceutical composition thereof.

In certain embodiments, a patient treated in accordance with the methods disclosed herein has a FEP of more than 50 μg/dl (e.g., more than or equal to 50 μg/dl, more than or equal to 60 μg/dl, more than or equal to 70 μg/dl, more than or equal to 80 μg/dl, more than or equal to 90 μg/dl, or more than or equal to 100 μg/dl) prior to administration of ferric citrate or a pharmaceutical composition thereof. In some embodiments, a patient treated in accordance with the methods disclosed herein has a PEP level of approximately 50 μg/dl, 60 μ8/d1, 70 μg/dl, 80 μg/dl, 90 μg/dl, or 100 μg/dl prior to administration of ferric citrate or a pharmaceutical composition thereof. In certain embodiments, a patient treated in accordance with the methods disclosed herein has a FEP level of approximately 50 μg/dl to approximately 60 μg/dl, approximately 50 μg/dl to approximately 70 μg/dl, approximately 50 μg/dl to approximately 80 μg/dl, approximately 50 μg/dl to approximately 90 μg/dl, approximately 50 μ8/dl to approximately 100 μg/dl, approximately 60 μg/dl to approximately 70 μg/d, approximately 60 μg/dl to approximately 80 μg/dl, approximately 60 μg/dl to approximately 90 μg/dl, approximately 60 μg/dl to approximately 100 μg/dl, approximately 70 μg/dl to approximately 80 μg/dl, approximately 70 μg/dl to approximately 90 μg/dl, approximately 70 μg/dl to approximately 100 μg/dl, approximately 80 μg/dl to approximately 90 μg/dl, approximately 80 μg/dl to approximately 100 μg/dl, or approximately 90 μg/dl to approximately 100 μg/dl prior to administration of ferric citrate or a pharmaceutical composition thereof.

In some embodiments, a patient treated in accordance with the methods disclosed herein has one, two, three or more, or all of the following prior to administration of ferric citrate or a pharmaceutical composition: (i) a hemoglobin concentration of less than or equal to approximately 12.5 grams/dl, 12 grams/dl or 11.5 grams/dl; (ii) a TSAT value of less than 50%, 45%, 40%, 35%, 30%, 25%, 20%, 15%, 12%, or 10%; (iii) a serum ferritin level of less than 300 ng/ml (e.g., less than or equal to 275 ng/ml, less than or equal to 250 ng/ml, less than or equal to 225 ng/ml, less than or equal to 200 ng/ml, less than or equal to 175 ng/ml, less than or equal to 150 ng/ml, less than or equal to 125 ng/ml, less than or equal to 100 ng/ml, less than or equal to 75 ng/ml, less than or equal to 50 ng/ml, less than or equal to 25 ng/ml, less than or equal to 15 ng/ml, less than or equal to 10 ng/ml, or less than or equal to 5 ng/ml); (iv) serum iron level of less than 6011 g/dl (e.g., less than or equal to 50 μg/dl, less than or equal to 40 μg/dl, less than or equal to 30 μg/dl, less than or equal to 20 μg/dl, or less than or equal to 10 μg/dl); (v) tissue iron level (e.g., stainable tissue iron level) of grade 2, grade 1, or grade 0, (vi) hematocrit level of less than 45%, 40%, 35%, 30%, 25%, 20%, 15%, or 10%; (vii) TIBC value of more than 390 μg/dl (e.g., more than or equal to 390 μg/dl, more than or equal to 400 μg/dl, more than or equal to 450 μg/dl, more than or equal to 450 μg/dl, more than or equal to 500 μg/dl, more than or equal to 550 μg/dl, more than or equal to 600 μg/dl, more than or equal to 650 μg/dl, more than or equal to 700 μg/dl, more than or equal to 800 μg/dl, more than or equal to 900 μg/dl, more than or equal to 1000 μg/dl, more than or equal to 1100 μg/dl, or more than or equal to 1200 μg/dl); (viii) plasma erythropoietin level of more than 20 mU/ml (e.g., more than or equal to 20 mU/ml, more than or equal to 25 mU/ml, more than or equal to 30 mU/ml, more than or equal to 40 mU/ml, more than or equal to 50 mU/ml, or more than or equal to 60 mU/ml); and/or (ix) FEP of more than 50 μg/dl (e.g., more than or equal to 50 μg/dl, more than or equal to 60 μg/dl, more than or equal to 70 μg/dl, more than or equal to 80 μg/dl, more than or equal to 90 μg/dl, or more than or equal to 100 μg/dl). In certain embodiments wherein the patient treated in accordance with the methods disclosed herein is a female, the patient has a TSAT value of less than 45%, 40%, 35%, 30%, 25%, 20%, 15%, or 12% prior to administration of ferric citrate or a pharmaceutical composition thereof. In certain embodiments wherein the patient treated in accordance with the methods disclosed herein is a male, the patient has a TSAT value of less than 50%, 45%, 40%, 35%, 30%, 25%, 20%, 15%, or 10% prior to administration of ferric citrate or a pharmaceutical composition thereof.

In certain embodiments, a patient treated in accordance with the methods disclosed herein has one, two, three or more, or all of the following prior to administration of ferric citrate or a pharmaceutical composition: (i) a hemoglobin concentration of approximately 6 grams/dl to approximately 8 grams/dl, approximately 6 grams/dl to approximately 10 grams/dl, approximately 6 grams/dl to approximately 12 grams/dl, approximately 7 grams/dl to approximately 9 grams/dl, approximately 7 grams/dl to approximately 11 grams/dl, approximately 7 grams/dl to approximately 13 grams/dl, approximately 8 grams/dl to approximately 10 grams/dl, approximately 8 grams/dl to approximately 12 grams/dl, approximately 9 grams/dl to approximately 11 grams/dl, approximately 9 grams/dl to approximately 12 grams/dl, approximately 4 grams/dl to approximately 13 grams/dl, approximately 10 grams/dl to approximately 11 grams/dl, approximately 10 grams/dl to approximately 12 grams/dl, approximately 10 grams/dl to approximately 13 grams/dl, approximately 11 grams/dl to approximately 12 grams/dl, approximately 11 grams/dl to approximately 13 grams/dl, or approximately 12 grams/dl to approximately 13 grams/dl; (ii) TSAT value of 10% to 45%, 12% to 45%, 20% to 45%, 20% to 40%, 10% to 35%, 20% to 25%, 15% to 50%, or 10% to 309% (iii) a serum ferritin level of approximately 5 ng/ml to approximately 15 ng/ml, approximately 5 ng/ml to approximately 25 ng/ml, approximately 5 ng/ml to approximately 50 ng/ml, approximately 15 ng/ml to approximately 25 ng/ml, approximately 15 ng/ml to approximately 50 ng/ml, approximately 15 ng/ml to approximately 75 ng/ml, approximately 25 ng/ml to approximately 50 ng/ml, approximately 25 ng/ml to approximately 75 ng/ml, approximately 25 ng/ml to approximately 100 ng/ml, approximately 50 ng/ml to approximately 75 ng/ml, approximately 50 ng/ml to approximately 100 ng/ml, approximately 50 ng/ml to approximately 150 ng/ml, approximately 75 ng/ml to approximately 100 ng/ml, approximately 75 ng/ml to approximately 150 ng/ml, approximately 100 ng/ml to approximately 150 ng/ml, approximately 150 ng/ml to approximately 200 ng/ml, approximately 150 ng/ml to approximately 250 ng/ml, approximately 100 ng/ml to approximately 300 ng/ml, approximately 200 ng/ml to approximately 300 ng/ml, or approximately 250 ng/ml to approximately 300 ng/ml; (iv) serum iron level of approximately 10 μg/dl to approximately 20 μg/dl, approximately 10 μg/dl to approximately 30 μg/dl, approximately 10 μg/dl to approximately 40 μg/dl, approximately 10 μg/dl to approximately 50 μg/dl, approximately 10 μg/dl to approximately 60 μg/dl, approximately 20 μg/dl to approximately 30 μg/dl, approximately 20 μg/dl to approximately 40 μg/dl, approximately 20 μg/dl to approximately 50 μg/dl, approximately 20 μg/dl to approximately 60 μg/dl, approximately 30 μg/dl to approximately 40 μg/dl, approximately 30 μg/dl to approximately 50 μg/dl, approximately 30 μg/dl to approximately 60 μg/dl, approximately 40 μg/dl to approximately 50 μg/dl, or approximately 40 μg/dl to approximately 60 μg/dl; (v) tissue iron level (e.g., stainable tissue iron level) of grade 2, grade 1, or grade 0; (vi) hematocrit level of 10% to 15%, 10% to 20%, 10% to 25%, 10% to 30%, 10% to 35%, 10% to 40%, 10% to 45%, 15% to 20%, 15% to 25%, 15% to 30%, 15% to 35%, 15% to 40%, 15% to 45%, 20% to 25%, 20% to 30%, 20% to 35%, 20% to 40%, 25% to 45%, 25% to 30%, 25% to 35%, 25% to 40%, 25% to 45%, 30% to 35%, 30% to 40%, 30% to 45%, 35% to 40%, 35% to 45%, or 40% to 45%; (vii) MC value of approximately approximately 39014/dl to approximately 600 μg/dl, approximately 390μg/dl to approximately 800 μg/dl, approximately 390 μg/dl to approximately 1000 μg/dl, approximately 390 μg/dl to approximately 1200 μg/dl, approximately 500 μg/dl to approximately 700 μg/dl, approximately 500 μg/dl to approximately 900 μg/dl, approximately 500 μg/dl to approximately 1100 μg/dl, approximately 600 μg/dl to approximately 800 μg/dl, approximately 600 μg/dl to approximately 1000 μg/dl, approximately 600 μg/dl to approximately 1200 μg/dl, approximately 700 μg/dl to approximately 900 μg/dl, approximately 700 μg/dl to approximately 1100 μg/dl, approximately 800 μg/dl to approximately 1000 μg/dl, approximately 800 μg/dl to approximately 1200 μg/dl, approximately 900 μg/dl to approximately 1100 μg/dl, or approximately 1000 μg/dl to approximately 1200 μg/dl; (viii) plasma erythropoietin level of approximately 20 mU/ml to approximately 30 mU/ml, approximately 20 mU/ml to approximately 40 mU/ml, approximately 20 mU/ml to approximately 50 mU/ml, approximately 20 mU/ml to approximately 60 mU/ml, approximately 30 mU/ml to approximately 40 mU/ml, approximately 30 mU/ml to approximately 50 mU/ml, approximately 30 mU/ml to approximately 60 mU/ml, approximately 40 mU/ml to approximately 50 mU/ml, approximately 40 mU/ml to approximately 60 mU/ml, or approximately 50 mU/ml to approximately 60 mU/ml; and/or (ix) FEP level of approximately 50 μg/dl to approximately 60 μg/dl, approximately 50 μg/dl to approximately 70 μg/dl, approximately 50 μg/dl to approximately 80 μg/dl, approximately 50 μg/dl to approximately 90 μg/dl, approximately 50 μg/dl to approximately 100 μg/dl, approximately 60 μg/dl to approximately 70 μg/dl, approximately 60 μg/dl to approximately 80 μg/dl, approximately 60 μg/dl to approximately 90 μg/dl, approximately 60 μg/dl to approximately 100 μg/dl, approximately 70 μg/dl to approximately 80 μg/dl, approximately 70 μg/dl to approximately 90 μg/dl, approximately 70 μg/dl to approximately 100 μg/dl, approximately 80 μg/dl to approximately 90 μg/dl, approximately 80 μg/dl to approximately 100 μg/dl, or approximately 90 mg/dl to approximately 100 μg/dl. In certain embodiments wherein the patient treated in accordance with the methods disclosed herein is a female, the patient has a TSAT value of 5% to 45%, 5% to 35%, 5% to 25%, 5% to 15%, 5% to 12%, 5% to 10%, 10% to 45%, 10% to 35%, 10% to 25%, 10% to 15%, 10% to 12%, 12% to 45%, 12% to 35%, 12% to 25%, 12% to 15%, 20% to 45%, 20% to 35%, 20% to 25%, 30% to 45%, 30% to 35%, or 40% to 45% prior to administration of ferric citrate or a pharmaceutical composition thereof. In certain embodiments wherein the patient treated in accordance with the methods disclosed herein is a male, the patient has a TSAT value of 5% to 50%, 5% to 40%, 5% to 30%, 5% to 20%, 5% to 15%, 5% to 10%, 10% to 50%, 10% to 4%, 10% to 30%, 10% to 20%, 10% to 15%, 15% to 50%, 15% to 40%, 15% to 30%, 15% to 25%, 15% to 20%, 20% to 50%, 20% to 40%, 20% to 30%, 20% to 25%, 30% to 50%, 30% to 40%, 30% to 35%, 40% to 50%, 40% to 45%, or 45% to 50% prior to administration of ferric citrate or a pharmaceutical composition thereof.

In certain embodiments, a patient treated in accordance with the methods disclosed herein has not taken a phosphate binder medication within 2 weeks, 3 weeks, 4 weeks, 1 month, 2 months, 3 months, 4 months, 5 months, 6 months or more of administration of the first dose of ferric citrate or a pharmaceutical composition thereof. In certain embodiments, a patient treated in accordance with the methods disclosed herein has not experienced acute kidney injury within 2 weeks, 3 weeks, 4 weeks, 5 weeks, 6 weeks, 1 month, 2 months, 3 months, 4 months, 5 months, 6 months or more of administration of the first dose of ferric citrate or a pharmaceutical composition thereof. In some embodiments, a patient treated in accordance with the methods disclosed herein has not been on dialysis or had a requirement for dialysis within 2 weeks, 3 weeks, 4 weeks, 5 weeks, 6 weeks, 1 month, 2 months, 3 months, 4 months, 5 months, 6 months or more of administration of the first dose of ferric citrate or a pharmaceutical composition thereof. In certain embodiments, a patient treated in accordance with the methods disclosed herein is not anticipated to require a kidney transplant or begin dialysis within 2 weeks, 3 weeks, 4 weeks, 5 weeks, 6 weeks, 1 month, 2 months, 3 months, 4 months, 5 months, 6 months, 7 months, 8 months or more of the first dose of ferric citrate or a pharmaceutical composition thereof.

In certain embodiments, a patient treated in accordance with the methods disclosed herein is not and/or has not received intravenous iron within 2 weeks, 3 weeks, 4 weeks, 5 weeks, 6 weeks, 1 month, 2 months, 3 months, 4 months, 5 months, 6 months or more of administration of the first dose of ferric citrate or a pharmaceutical composition thereof. In some embodiments, a patient treated in accordance with the methods disclosed herein is not and/or has not received an erythropoiesis-stimulating agent (ESA) within 2 weeks, 3 weeks, 4 weeks, 5 weeks, 6 weeks, 1 month, 2 months, 3 months, 4 months, 5 months, 6 months or more of administration of the first dose of ferric citrate or a pharmaceutical composition thereof in certain embodiments, a patient treated in accordance with the methods disclosed herein is not and/or has not received intravenous iron and an erythropoiesis-stimulating agent (ESA) within 2 weeks, 3 weeks, 4 weeks, 5 weeks, 6 weeks, 1 month, 2 months, 3 months, 4 months, 5 months, 6 months or more of administration of the first dose of ferric citrate or a pharmaceutical composition thereof. In some embodiments, a patient treated in accordance with the methods disclosed herein is not receiving intravenous iron and/or an erythropoiesis-stimulating agent (ESA).

In certain embodiments, a patient treated in accordance with the methods disclosed herein has and/or has been diagnosed IDA associated with one, two or more of the following conditions: chronic blood loss; acute blood loss; childbirth; menstruation; menorrhagia; dialysis; chronic kidney Disease (CKD); dysfunctional uterine bleeding; heavy uterine bleeding; urinary tract bleeding; hemoglobinuria; chronic internal bleeding; gastrointestinal bleeding; angiodysplasia; idiopathic pulmonary hemosiderosis; blood loss from injury, surgery, acute trauma, or frequent blood drawing; bleeding ulcer; gastric ulcer; duodenal ulcer; intravascular hemolysis; chronic recurrent hemoptysis; colon polyp; gastrointestinal cancer (such as colonic cancer, gastric cancer, and intestinal cancer); gastrointestinal disorder (e.g., inflammatory bowel disease (IBD) and Crohn's disease); celiac disease; post surgical bowel resection; gut resection or bypass; Whipple's disease; chronic heart failure; systemic inflammation; parasitic infections (such as malaria and infections with hookworms, tapeworms, flukes, whipworms, roundworms, T. trichiura, or N. Pylori); and/or pregnancy. In some embodiments, a patient treated in accordance with the methods disclosed herein has IDA associated with the use of proton pump inhibitors; use of antacids; use of non-steroidal anti-inflammatory, drugs (NSAIDs) (e.g., aspirin, anticoagulants such as clopidogrel and warfarin); chronic ingestion of alcohol; chronic ingestion of salicylates; chronic ingestion of steroids; chronic ingestion of non-steroidal anti-inflammatory agents; chronic ingestion of erythropoiesis stimulating agents; insufficient dietary intake of iron and/or insufficient absorption of iron; deficient levels of hemoglobin; childhood development; psychomotor and cognitive development in children; and/or breath holding spells.

Insufficient dietary intake of iron, blood loss in women, and infectious diseases are also major causes of IDA. In certain embodiments, a patient treated in accordance with the methods disclosed herein has and/or has been diagnosed with IDA associated with insufficient dietary intake of iron. In some embodiments, a patient treated in accordance with the methods disclosed herein has and/or has been diagnosed with IDA associated with insufficient absorption of iron. In certain embodiments, a patient treated in accordance with the methods disclosed herein has and/or has been diagnosed with IDA associated with insufficient dietary intake of iron and/or insufficient absorption of iron. In some embodiments, a patient treated in accordance with the methods disclosed herein has and/or has been diagnosed with IDA associated with menstruation. In some embodiments, a patient treated in accordance with the methods disclosed herein has and/or has been diagnosed with IDA associated with child birth. In some embodiments, a patient treated in accordance with the methods disclosed herein has and/or has been diagnosed with IDA s associated with an infection with hookworms. In some embodiments, a patient treated in accordance with the methods disclosed herein has and/or has been diagnosed with IDA associated with malaria.

In some embodiments, a patient treated in accordance with the methods disclosed herein has and/or has been diagnosed with IDA associated with one, two or more of the following conditions: gastrointestinal bleeding, angiodysplasia; gastric ulcer; duodenal ulcer; colon polyp; gastrointestinal cancer (such as colonic cancer, gastric cancer, and intestinal cancer); gastrointestinal disorder (e.g., inflammatory bowel disease (IBD) and Crohn's disease); celiac disease; post surgical bowel resection; gut resection or bypass; and Whipple's disease. In specific embodiments, a patient treated in accordance with the methods disclosed herein has and/or has been diagnosed with IDA associated with gastrointestinal cancer (such as colonic cancer, gastric cancer, and intestinal cancer).

In certain embodiments, a patient treated in accordance with the methods disclosed herein has and/or has been diagnosed with a gastrointestinal condition. In some embodiments, a patient treated in accordance with the methods disclosed herein has and/or has been diagnosed with inflammatory bowel disease, inflammatory bowel syndrome, ulcerative colitis, Crohn's disease, microscopic colitis (such as collagenous or lymphocytic colitis), and/or chemically-induced colitis (e.g., NSAID-induced colitis). In certain embodiments, a patient treated in accordance with the methods disclosed herein has gastrointestinal bleeding. In specific embodiments, a patient treated in accordance with the methods disclosed herein has gastrointestinal bleeding associated with a gastrointestinal condition, such as inflammatory bowel disease, inflammatory bowel syndrome, Crohn's disease, ulcerative colitis, microscopic colitis (such as collagenous or lymphocytic colitis), or chemically-induced colitis (e.g., NSAID-induced colitis).

In certain embodiments, a patient treated in accordance with the methods disclosed herein has not received a blood transfusion within 2 weeks, 3 weeks, 4 weeks, 5 weeks, 6 weeks, or more of initiating administration of ferric citrate. In other embodiments, a patient treated in accordance with the methods disclosed herein has received a blood transfusion within 2 weeks, 3 weeks, 4 weeks, 5 weeks, 6 weeks, or more of initiating administration of ferric citrate.

In certain embodiments, a patient treated in accordance with the methods disclosed herein has not been diagnosed with a malignancy within 1 month, 3 months, 6 months, 1 year, 2 years, 3 years, 4 years, 5 year or 6 years of initiating administration of ferric citrate. In other embodiments, a patient treated in accordance with the methods disclosed herein has been diagnosed with a malignancy. In some embodiments, a patient treated in accordance with the methods disclosed herein has not been diagnosed with hemochromatosis. In other embodiments, a patient treated in accordance with the methods disclosed herein has been diagnosed with hemochromatosis. In specific embodiments, a patient treated in accordance with the methods disclosed herein has no known allergies to iron products and/or a previous intolerance to oral ferric citrate.

In specific embodiments, a patient treated in accordance with the methods disclosed herein fulfills one, two, three or more of the inclusion criteria in Section 5, infra and/or does not fulfill one, two, three or more of the exclusion criteria in Section 5, infra.

### 4.3. Dosing and Administration

In one aspect in accordance with the methods disclosed herein, the ferric citrate or a pharmaceutical composition thereof is administered to a subject as frequently as necessary and/or desired to treat the IDA. In some embodiments in accordance with the methods disclosed herein, the ferric citrate or a pharmaceutical composition thereof is administered to a subject once per day. In certain embodiments in accordance with the methods disclosed herein, the ferric citrate or a pharmaceutical composition thereof is administered to a subject twice per day. In some embodiments in accordance with the methods disclosed herein, the ferric citrate or a pharmaceutical composition thereof is administered to a subject three times per day. In specific embodiments in accordance with the methods disclosed herein, the ferric citrate or a pharmaceutical composition thereof is administered orally to a subject.

In various aspects, the daily dose of ferric citrate or a pharmaceutical composition thereof administered to a subject is split up during the course of a single day. By way of example, a single daily dose of ferric citrate may be 6 grams and that 6 grams may be spread out over the course of the day such that 2 grams is taken in the morning, 2 grams is taken in the afternoon, and the final 2 grams is taken in the evening, for a total of 6 grams over the course of a day.

Pharmaceutical compositions, such as tablets and other oral dosage forms, disclosed herein can be made to accommodate a number of doses of ferric citrate. Pharmaceutical compositions comprising ferric citrate which may be administered to a subject are described in Section 4.5, infra. In certain embodiments, the weight of an individual tablet or other oral dosage form depends upon the final dosage to be produced; e.g., 125 mg, 250 mg, 500 mg, 667 mg, 750 mg and 1,000 mg of ferric citrate per tablet. In a specific embodiment, the ferric citrate is provided in a tablet dosage form comprising approximately 1 gram of ferric citrate equivalent to approximately 210 mg of ferric iron. The number of tablets or other oral dosage forms administered to a subject can be adjusted to conform to the desired amount of ferric citrate to be administered. For example, if a subject is directed to take 4 grams of ferric citrate daily in a single dose, the subject may take 4 tablets or other oral dosage forms, each comprising 1 gram of ferric citrate, or may take 8 tablets or other oral dosage forms, each comprising 500 mg of ferric citrate.

In some embodiments, a daily dose of ferric citrate administered to a subject in accordance with the methods disclosed herein is from 1 gram to 12 grams, at a dose of ferric iron ranging from 210 mg to 2, 520 mg. In some embodiments, one or more tablets comprising 1 gram of ferric citrate, each tablet having a dose of ferric iron of 210 mg, is/are administered to a subject in accordance with the methods disclosed herein.

In some embodiments, the ferric citrate is administered to a subject in accordance with the methods disclosed herein at a daily dose of 1 tablet per day, the tablet comprising 1 gram of ferric citrate containing 210 mg of ferric iron, for a total daily dose of 1 gram of ferric citrate and 210 mg ferric iron. In certain embodiments, the ferric citrate is administered to a subject in accordance with the methods disclosed herein at a daily dose of 2 tablets per day, each tablet comprising 1 gram of ferric citrate containing 210 mg of ferric iron, for a total daily dose of 2 grams of ferric citrate and 420 mg ferric iron. In some embodiments, the ferric citrate is administered to a subject in accordance with the methods disclosed herein at a daily dose of 3 tablets per day, each tablet comprising 1 gram of ferric citrate containing 210 mg of ferric iron, for a total daily dose of 3 grams of ferric citrate and 630 mg ferric iron. In certain embodiments, the ferric citrate is administered to a subject in accordance with the methods disclosed herein at a daily dose of 4 tablets per day, each tablet comprising 1 gram of ferric citrate containing 210 mg of ferric iron, for a total daily dose of 4 grams of ferric citrate and 840 mg ferric iron. In some embodiments, the ferric citrate is administered to a subject in accordance with the methods disclosed herein at a daily dose of 5 tablets per day, each tablet comprising 1 gram of ferric citrate containing 210 mg of ferric iron, for a total daily dose of 5 grams of ferric citrate and 1,050 mg ferric iron. In certain embodiments, the ferric citrate is administered to a subject in accordance with the methods disclosed herein at a daily dose of 6 tablets per day, each tablet comprising 1 gram of ferric citrate containing 210 mg of ferric iron, for a total daily dose of 6 grams of ferric citrate and 1,260 mg ferric iron. In some embodiments, the ferric citrate is administered to a subject in accordance with the methods disclosed herein at a daily dose of 7 tablets per day, each tablet comprising 1 gram of ferric citrate containing 210 mg of ferric iron, for a total daily dose of 7 grams of ferric citrate and 1,470 mg ferric iron. In certain embodiments, the ferric citrate is administered to a subject in accordance with the methods disclosed herein at a daily dose of 8 tablets per day, each tablet comprising 1 gram of ferric citrate containing 210 mg of ferric iron, for a total daily dose of 8 grams of ferric citrate and 1,680 mg ferric iron. In some embodiments, the ferric citrate is administered to a subject in accordance with the methods disclosed herein at a daily dose of 9 tablets per day, each tablet comprising 1 gram of ferric citrate containing 210 mg of ferric iron, for a total daily dose of 9 grams of ferric citrate and 1,890 mg ferric iron. In certain embodiments, the ferric citrate is administered to a subject in accordance with the methods disclosed herein at a daily dose of 10 tablets per day, each tablet comprising 1 gram of ferric citrate containing 210 mg of ferric iron, for a total daily dose of 10 grams of ferric citrate and 2,100 mg ferric iron. In some embodiments, the ferric citrate is administered to a subject in accordance with the methods disclosed herein at a daily dose of 11 tablets per day, each tablet comprising 1 gram of ferric citrate containing 210 mg of ferric iron, for a total daily dose of 11 grams of ferric citrate and 2,310 mg ferric iron. In some embodiments, the ferric citrate is administered to a subject in accordance with the methods disclosed herein at a daily dose of 12 tablets per day, each tablet comprising 1 gram of ferric citrate containing 210 mg of ferric iron, for a total daily dose of 12 grams of ferric citrate and 2,520 mg ferric iron. Tablets which may be administered to a subject are described in Section 4.5, infra. In a specific embodiment, the tablet is Auryxia™ (Ferric Citrate; Keryx Biopharmaceuticals, Inc.).

In a specific aspect, each dose of ferric citrate administered to a subject in accordance with the methods is without food. In certain embodiments in accordance with the methods disclosed herein, each dose of ferric citrate is administered to a subject approximately 1 hour prior to the intake of food. In some embodiments in accordance with the methods disclosed herein, each dose of ferric citrate is administered to a subject approximately 2 hours prior to the intake of food. In certain embodiments in accordance with the methods disclosed herein, each dose of ferric citrate is administered to a subject approximately 3 hours prior to the intake of food. In some embodiments in accordance with the methods disclosed herein, each dose of ferric citrate is administered to a subject approximately 4 hours prior to the intake of food. In certain embodiments in accordance with the methods disclosed herein, each dose of ferric citrate is administered to a subject approximately 1-2, 1-3, 1-4, 2-3, 2-4, or 3-4 hours prior to the intake of food. In accordance with these embodiments, the ferric citrate can be administered as a pharmaceutical composition, such as described in Section 4.5, infra.

In certain embodiments in accordance with the methods disclosed herein, each dose of ferric citrate is administered to a subject approximately 1 hour after the intake of food. In some embodiments in accordance with the methods disclosed herein, each dose of ferric citrate is administered to a subject approximately 2 hours after the intake of food. In certain embodiments in accordance with the methods disclosed herein, each dose of ferric citrate is administered to a subject approximately 3 hours after the intake of food. In some embodiments in accordance with the methods disclosed herein, each dose of ferric citrate is administered to a subject approximately 4 hours after the intake of food. In certain embodiments in accordance with the methods disclosed herein, each dose of ferric citrate is administered to a subject approximately 1-2, 1-3, 1-4, 2-3, 2-4, or 3-4 hours after the intake of food. In accordance with these embodiments, the ferric citrate can be administered as a pharmaceutical composition, such as described in Section 4.5, infra.

In some embodiments in accordance with the methods disclosed herein, no food is ingested by a subject within approximately 1 hour of the administration each dose of ferric citrate. In certain embodiments in accordance with the methods disclosed herein, no food is ingested by a subject within approximately 2 hours of the administration each dose of ferric citrate. In some embodiments in accordance with the methods disclosed herein, no food is ingested by a subject within approximately 3 hours of the administration each dose of ferric citrate. In certain embodiments in accordance with the methods disclosed herein, no food is ingested by a subject within approximately 4 hours of the administration each dose of ferric citrate. In some embodiments in accordance with the methods disclosed herein, no food is ingested by a subject within approximately 1-2, 1-3, 1-4, 2-3, 2-4, or 3-4 hours of the administration each dose of ferric citrate. In accordance with these embodiments, the ferric citrate can be administered as a pharmaceutical composition, such as described in Section 4.5, infra.

In one embodiment, the ferric citrate is administered to a subject at the dose(s) described in the Examples in Section 5, infra, in a specific embodiment, the ferric citrate is administered to a subject at the dose(s) and in the tablet form described in the Examples in Section 5, infra. In another specific embodiment, the dose of ferric citrate administered to a subject in accordance with the methods disclosed herein is not sufficient to treat hyperphosphatemia.

The ferric citrate or a pharmaceutical composition thereof can be administered for any length of time, such as, e.g., the length of time prescribed by a medical professional (e.g., a doctor, nurse practitioner or physician assistant). In any of the methods described herein, ferric citrate or a pharmaceutically acceptable composition thereof can be administered to the patient for a long period of time, for example, up to and including 52 weeks, including up to and including 56 weeks. The ferric citrate may also be administered to the patient for a short period of time, for example, 2 weeks, 4 weeks, 6 weeks, 8 weeks, 9 weeks, 10 weeks, or 12 weeks.

### 4.4. Combination Therapy

In certain embodiments, ferric citrate or a pharmaceutical composition thereof described herein may be administered or applied singly, or in combination with other agents. Ferric citrate or a pharmaceutical composition thereof described herein may also be administered or applied singly or in combination with other pharmaceutically active agents, including other agents known to improve one or more iron storage parameters (e.g., increase serum ferritin level, increase transferrin saturation (TSAT), increase hemoglobin concentration, increase serum iron level, increase tissue iron level (e.g., stainable tissue iron level), increase TIBC value, increase plasma erythropoietin level, increase FEP level), increase iron absorption, maintain iron stores, treat iron deficiency, or treat anemia. In specific embodiments, ferric citrate or a pharmaceutical composition thereof described herein is not administered in combination with other pharmaceutically active agents known to improve one or more iron storage parameters (e.g., increase serum ferritin levels, increase transferrin saturation (TSAT), increase hemoglobin concentration, increase serum iron level, increase tissue iron level (e.g., stainable tissue iron level), increase TIBC value, increase plasma erythropoietin level, increase FEP level), increase iron absorption, maintain iron stores, treat iron deficiency, or treat anemia. For example, in specific embodiments, ferric citrate or a pharmaceutical composition thereof described herein is not administered in combination with one, two or all of the following: erythropoiesis-stimulating agent(s), intravenous iron and/or a blood transfusion.

As used herein, “in combination” in the context of the administration of agents or therapies refers to the use of more than one agent or therapy. The use of the term “in combination” does not restrict the order in which agents or therapies are administered to a patient with a disease. In certain embodiments, administration of one or more agents or therapies to a patient with a disease includes, without limitation, a first agent or therapy that can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 1.2 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second agent or therapy to a patient which had, has, or is susceptible to a disease.

In certain embodiment, ferric citrate or a pharmaceutical composition thereof described herein is administered in combination with a pharmaceutically active agent known to treat a gastrointestinal condition, such as colitis or inflammatory bowel disease, or agents known to ameliorate one or more symptoms thereof. For example, in some embodiments, ferric citrate or a pharmaceutical composition thereof described herein is administered in combination with an anti-inflammatory drug (e.g., aminosalicylate or corticosteroid), an immunosuppressent (e.g., azathioprine (Azasan, Imuran), mercaptopurine, cyclosporine, infliximab (Remicade®), adalimumab (Humira®), golimumab (Simponi®), vedolizumab (Entyvio®)), antibiotics, anti-diarrheal agents and/or pain relievers. The ferric citrate and an additional agent(s) may be combined in any manner known in the art such as a unitary dosage form. Alternatively, the ferric citrate and an additional agent(s) may be administered to a subject in separate dosage forms intended for simultaneous or sequential administration to the subject. When administered sequentially, the combination may be administered in two or more administrations. In certain embodiments, the ferric citrate or a pharmaceutical composition thereof described herein and one or more additional agents are administered by different routes. In other embodiments, the ferric citrate or a pharmaceutical composition thereof described herein and one or more additional agents are administered by the same route.

### 4.5. Ferric Citrate

Disclosed herein are preparations of ferric citrate and pharmaceutical compositions comprising the ferric citrate for use in accordance with the methods described herein. In various embodiments, the ferric citrate preparations, and the pharmaceutical compositions comprising the ferric citrate preparations, meet certain dissolution, tableting and disintegration standards. In various aspects, the pharmaceutical compositions can include ferric citrate as the active ingredient and a binder. The pharmaceutical compositions also can include a lubricant and/or a disintegrant (which, in some embodiments, can be the same as the binder).

In certain embodiments, the ferric citrate used as described herein is disclosed in U.S. Pat. Nos. 7,767,851, 8,093,423, 8,299,298, 8,338,642, 8,754,258, 8,846,976, and/or 8,754,257, and/or International Patent Publication Nos. WO 2004/074444, WO 2007/022435, WO 2007/089571, WO 2007/089577 and/or WO 2011/011541. In some embodiments, the ferric citrate used as described herein has certain characteristics or features of the ferric citrate disclosed in U.S. Pat. Nos. 7,767,851, 8,093,423, 8,299,298, 8,338,642, 8,754,258, 8,846,976, and/or 8,754,257, and/or International Patent Publication Nos. WO 2004/074444, WO 2007/022435, WO 2007/089571, WO 2007/089577 and/or WO 2011/011541.

In specific aspects, the ferric citrate used as described herein display an enhanced BET active surface area compared to commercially available or chemical grade forms of ferric citrate. BET theory explains the physical adsorption of gas molecules onto a solid surface. The theory serves as the basis for the measurement of the specific surface area of a material. This theory allows the calculation of surface areas of materials in a very accurate manner and is thus capable of distinguishing differences between separate preparations of what would otherwise appear to be the same material. For example, activated carbon is a form of carbon that has been processed to make it extremely porous and thus to have a very large surface area. Activated carbon has been experimentally determined, using calculations derived from BET theory, to have a surface area of around 3000 m² g⁻¹. This surface area is significantly higher than the active surface areas of other preparations of carbon even though they are made of the same material.

In some embodiments, the ferric citrate used as described herein has a BET active surface area exceeding 16 m²/g. In certain embodiments, the ferric citrate used in accordance with the methods described herein has a BET active surface area exceeding 20 m²/g. In some embodiments, the ferric citrate used as described herein has a BET active surface area exceeding 25 m²/g. In certain embodiments, the ferric citrate used as described herein has a BET active surface area exceeding 30 m²/g. In some embodiments, the ferric citrate used as described herein has a BET active surface area exceeding 35 m²/g. In certain embodiments, the ferric citrate used as described herein has a BET active surface area exceeding 40 m²/g. In some embodiments, the ferric citrate used as described herein has a BET active surface area exceeding 45 m²/g. In certain embodiments, the ferric citrate used as described herein has a BET active surface area exceeding 50 m²/g.

In some embodiments, the ferric citrate used as described herein have a BET active surface area ranging from 16.17 m²/g to 19.85 m/g. In certain embodiments, the ferric citrate used as described herein has a BET active surface area selected from 16.17 m²/g and 19.85 m²/g. In some embodiments, the ferric citrate used as described herein has a BET active surface area exceeding 27 m²/g. In some embodiments, the ferric citrate used as described herein have a BET active surface area ranging from 27.99 m²/g to 32.34 m²/g. In some embodiments, the ferric citrate used as described herein have a BET active surface area ranging from 28.5 m/g to 31.5 m²/g. In some embodiments, the ferric citrate used as described herein have a BET active surface area selected from 27.99 m²/g, 28.87 m²/g and 3234 m²/g. In some embodiments, the ferric citrate used as described herein have a BET active surface area selected from 28.5 m²/g, 29.1 m²/g, 30.6 m²/g and 31.5 m²/g. In some embodiments, the ferric citrate preparations used as described herein have a BET active surface area from 30 m²/g to 40 m²/g. In some embodiments, the ferric citrate preparations used as described herein have a BET active surface area from 20 m²/g to 35 m²/g.

In certain embodiments, the ferric iron content of the ferric citrate is greater than or exceeds about 19% w/w. In some embodiments, the ferric iron content of the ferric citrate is 21.2% w/w, 22.1% w/w, or 22.4% w/w. In certain embodiments, the ferric iron content of the ferric citrate is between 19.5% w/w and 22.5%. In certain embodiments, the ferric iron content of the ferric citrate is between 21% w/w and 23% w/w. Techniques known to one of skill in the art can be used to determine the iron content of ferric citrate. In a specific embodiment, the ferric iron content is determined as follows: Pre-weighed ferric citrate is mixed with an appropriate amount of water and an appropriate amount of hydrochloric acid. The mixture is heated to boiling, and then cooled. Solid potassium iodide is added into the mixture, and the solution turns to dark-red and almost brown. A sample is removed from the solution and titrated with sodium thiosulfate until the sample turns to olive-green, when starch solution is added, and the sample then turns to blue-black. Titration with sodium thiosulfate is continued until the blue-black color disappears. Iron content is then calculated using the weight of ferric citrate, the pre determined titer of sodium thiosulfate, and the total volume of sodium thiosulfate added.

In a specific embodiment, the ferric citrate used as described herein is a complex comprising iron (III) and citric acid. In specific aspects, the complex of iron (III) and citric acid comprises water. In some embodiments, the molar ratio of iron (III) to citric acid is from 1:0.70 to 1:0.78. In some aspects, the molar ratio of iron (III) to citric acid is from 1:0.69 to 1:0.87. In certain embodiments, the molar ratio of iron (III) to citric acid is from 1:0.75 to 1:1.10. In some embodiments, the molar ratio of iron (III) to citric acid is from 1:0.78 to 1:0.95. In certain embodiments, the molar ratio of iron (III) to citric acid is from 1:0.80 to 1:0.92. In some embodiments, the molar ratio of iron (III) to citric acid is from 1:0.81 to 1:0.91. In certain embodiments, the molar ratio of iron (III) to citric acid is from 1:0.75 to 1:1.15. In some embodiments, the molar ratio of iron (III) to citric acid is from 1:0.80 to 1:1.10.

In some embodiments, the molar ratio of iron (III) to water is from 1:0.32 to 1:0.42. In certain embodiments, the molar ratio of iron (III) to water is from 1:0.32 to 1:0.46. In some aspects, the molar ratio of iron (III) to water is from 1:1.8 to 1:3.2. In some embodiments, the molar ratio of iron (III) to water is from 1:1.8 to 1:3.2. In certain embodiments, the molar ratio of iron (III) to water is from 1:2.4 to 1:3.1. In some embodiments, the molar ratio of iron (III) to water is from 1:2.7 to 1:3.1.

In a specific embodiment, the ferric citrate used as described herein is known chemically as iron (+3), x (1, 2, 3-propanetricarboxylic acid, 2-hydroxy-), y (H₂O)

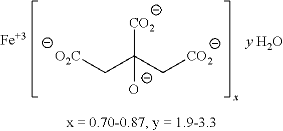

In specific embodiments, the ferric citrate used as described herein is tetraferric tricitrate decahydrate.

In specific embodiments, the ferric citrate used as described herein is substantially free of impurities, such as beta-iron hydroxide oxide. In particular embodiments, the ferric citrate used as described herein contains less than 6% of impurities, such as beta-iron hydroxide oxide, by weight based on the total weight of the Ionic citrate. In some embodiments, the ferric citrate used as described herein contains less than 5% of impurities, such as beta-iron hydroxide oxide, by weight based on the total weight of the ferric citrate. In certain embodiments, the ferric citrate used as described herein contains less than 4% of impurities, such as beta-iron hydroxide oxide, by weight based on the total weight of the ferric citrate. In some embodiments, the ferric citrate used as described herein contains less than 3% of impurities, such as beta-iron hydroxide oxide, by weight based on the total weight of the ferric citrate.

In specific aspects, the ferric citrate used as described herein is more soluble compared to commercially available or chemical grade forms of ferric citrate. In specific embodiments, in dissolution testing, the percentage of ferric citrate dissolved within 5 minutes is 91% or more, within 15 minutes is 96% or more, within 30 minutes is 96% or more and within 60 minutes is 95% or more in dissolution testing conducted on the ferric citrate preparations in USP <711> vessels using Apparatus II. The particular standard used for the dissolution testing establishes a baseline of 100 so to the extent that a batch may have a dissolution greater than 100%, it is a dissolution rate relative to that standard.

In some embodiments, 80% or more of the ferric citrate used as described herein is dissolved within 15 minutes in dissolution testing conducted in LISP <711> vessels using Apparatus II. In certain embodiments, 85% or more of the ferric citrate used as described herein is dissolved within 15 minutes in dissolution testing conducted in USP <<711> vessels using Apparatus it. In some embodiments, 90% or more of the ferric citrate used as described herein is dissolved within 15 minutes in dissolution testing conducted in USP <711> vessels using Apparatus II. In certain embodiments, 91% or more of the ferric citrate used as described herein is dissolved within 15 minutes in dissolution testing conducted in USP <711> vessels using Apparatus II. In some embodiments, 95% or more of the ferric citrate used as described herein is dissolved within 15 minutes in dissolution testing conducted in USP <711> vessels using Apparatus II. In certain embodiments, 96% or more of the ferric citrate used as described herein is dissolved within 15 minutes in dissolution testing conducted in USP <711> vessels using Apparatus II. In some embodiments, 97% or more of the ferric citrate used as described herein is dissolved within 15 minutes in dissolution testing conducted in USP <711> vessels using Apparatus II. In certain embodiments, 100% of the ferric citrate used as described herein is dissolved within 15 minutes in dissolution testing conducted in USP <711> vessels using Apparatus II.

Without being bound by any theory, the increase in solubility of the ferric citrate is believed to be a result of the unique, significantly large active surface area of the ferric citrate. The intrinsic dissolution rate is defined as the dissolution rate of pure substances under the condition of constant surface area. The intrinsic dissolution rate and bioavailability of a drug substance is influenced by its solid state properties including: crystallinity, amorphism, polymorphism, hydration, solvation, particle size and particle surface area. The measured intrinsic dissolution rate is dependent on these solid-state properties and is typically determined by exposing a constant surface area of a material to an appropriate dissolution medium while maintaining constant temperature, stirring rate, and pH.

In some embodiments, the ferric citrate used as described herein has an intrinsic dissolution rate of between 1.88 mg/cm²/min to 4 mg/cm²/min. in certain embodiments, the ferric citrate used as described herein has an intrinsic dissolution rate of greater than 2.28 mg/cm²/min. in some embodiments, the ferric citrate used as described herein has an intrinsic dissolution rate exceeding 2.28 mg/cm²/min. In certain embodiments, the ferric citrate used as described herein has an intrinsic dissolution rate of 2.99 mg/cm²/min. In some embodiments, the ferric citrate used as described herein has an intrinsic dissolution rate ranging from 2.28 mg/cm²/min to 2.99 mg/cm²/min. In certain embodiments, the ferric citrate used as described herein has an intrinsic dissolution rate selected from 2.28 mg/cm²/min and 2.99 mg/cm²/min. In specific embodiments, the commercial grade preparations of ferric citrate have an intrinsic dissolution rate that is substantially lower than the ferric citrate described herein.

Exemplary methods of manufacture of preparations of the ferric citrate are disclosed in U.S. Pat. Nos. 7,767,851, 8,093,423, 8,299,298, 8,338,642, 8,754,258, 8,846,976, and 8,754,257, U.S. Publication No. 2012/0238622 and International Publication Nos. WO 2004/074444, WO 2007/022435, WO 2007/089571, WO 2007/089577 and WO 2011/011541.

### 4.5.1. Pharmaceutical Composition of Ferric Citrate

In a specific embodiment, the ferric citrate is contained in a pharmaceutical composition. In one embodiment, a pharmaceutical composition comprises ferric citrate and a pharmaceutically acceptable excipient or carrier. In a particular embodiment, a pharmaceutical composition comprises ferric citrate and a binder. In some embodiments, the pharmaceutical compositions further comprise a lubricant and/or a disintegrant (which, in certain embodiments, can be the same as the binder). In a specific embodiment, the pharmaceutical compositions include ferric citrate as the active ingredient. In some embodiments, the pharmaceutical compositions are oral tablet dosage forms. In certain embodiments, the pharmaceutical compositions are oral formulations other than tablets, such as capsules, suspensions, syrups, or sachets. In specific embodiment, the ferric citrate used in the pharmaceutical compositions is one or more forms of the ferric citrate described in Section 4.5, infra. In a specific embodiment, the ferric citrate used in a pharmaceutical composition described herein is known chemically as iron (+3), x (1, 2, 3-propanetricarboxylic acid, 2-hydroxy-), y (H₂O) e x Y H₂O,

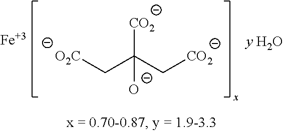

In specific embodiments, the ferric citrate used in a pharmaceutical composition described herein is tetraferric tricitrate decahydrate.

The pharmaceutical compositions described herein may be utilized in the methods described herein.

In some embodiments, the pharmaceutical compositions and oral tablet dosage forms provided by this disclosure are disclosed in International Publication No. WO 2011/011541 and U.S. Publication No. 2012/0115945.

1n a specific aspect, the pharmaceutical compositions are tablets or other oral formulations that include ferric citrate and a binder. In some embodiments, the tablets or other oral formulations can include ferric citrate, a binder, a lubricant and a disintegrant. In a specific embodiment, a single tablet comprises 1 gram of ferric citrate having a 210 mg dose of ferric iron.

In some embodiments, the tablets or other oral formulations are characterized as highly drug loaded with the ferric citrate present in the tablets at values of greater than approximately 65% by weight of the formulation, greater than approximately 70% by weight of the formulation, greater than approximately 75% by weight of the formulation, greater than approximately 80% by weight of the formulation, greater than approximately 85% by weight of the formulation, greater than approximately 90% by weight of the formulation and as high as approximately 92% or approximately 95% of the formulation. Intermediate values such as approximately 80% by weight ferric citrate, approximately 85% by weight ferric citrate and approximately 90% by weight ferric citrate also can be used in the ferric citrate tablets or other oral formulations. In some embodiments, the tablets or other oral formulations are characterized as highly drug loaded with the ferric citrate present in the tablets at values of approximately 75% to approximately 92%, approximately 80% to approximately 92%, approximately 85% to approximately 92%, approximately 80% to approximately 90%, approximately 85% to approximately 90%, approximately 90% to approximately 92%, approximately 80% to approximately 95%, approximately 85% to approximately 95%, or approximately 90% to approximately 95% The characteristics of the tablets produced at these highly loaded weight percentages may be controlled by variables such as binder, binder amount, disintegrant, disintegrant amount, formulation method used (e.g., granulation, direct compression), tableting parameters, etc. Thus if a tablet is made and it has a slight amount of lamination or capping, by varying one or more of the above variables, the lamination or capping can be corrected.

In various embodiments, the tablets or other oral formulations comprise ferric and one or more components selected from among one or more binders, one or more lubricants, and one or more disintegrants. In certain embodiments, the tablets or other oral formulations comprise ferric citrate and one or more binders. In some embodiments, the tablets or other oral formulations comprise ferric citrate, one or more binders, and one or more lubricants. In certain embodiments, the tablets or other oral formulations comprise ferric citrate, one or more binders, one or more lubricants, and one or more disintegrants.

Any binder known to one skilled in the art may be used in the tablets or other oral formulations described herein. In certain embodiments, the binder is hydroxypropyl cellulose (HPC), hydroxypropylmethyl cellulose (HPMC), sodium alginate, alginic acid, guar gum, acacia gum, xanthan gum, carbolpol, cellulose gum (carboxy methyl cellulose), ethyl cellulose, maltodextrin, PVP/VA, povidone, microcrystalline cellulose, starch, partially or fully pregelatinized starch, or methyl cellulose. In some embodiments, the tablet or other oral formulation comprises a combination of two or more of the following binders: comprises hydroxypropyl cellulose (HPC), hydroxypropylmethyl cellulose (HPMC), sodium alginate, alginic acid, guar gum, acacia gum, xanthan gum, carbolpol, cellulose gum (carboxy methyl cellulose), ethyl cellulose, maltodextrin, PVP/VA, povidone, microcrystalline cellulose, starch, partially or fully pregelatinized starch, or methyl cellulose. The maltodextrin, PVP/VA, and methyl cellulose function as immediate release binders when used in the ferric citrate tablets or other oral formulations. In a specific embodiment, the binder used in a tablet or other oral formulation comprises partially or fully pregelatinized starch.

It also should be understood that combinations of binders can be used to control and vary the effect of the binder. For example, a binder system can be made up of hydroxypropyl cellulose and polyvinyl pyrrolidone (povidone) with or without microcrystalline cellulose. One or both of the hydroxypropyl cellulose and povidone can be replaced with pregelatinized starch.

In various aspects, the tablets or other oral formulations can include a lubricant. Any lubricant known to one skilled in the art can be used in the tablets or other oral formulations. In certain embodiments, the lubricant used in the ferric citrate tablets or other oral formulations is magnesium stearate, calcium stearate, sodium stearyl fumarate. In some embodiments, the ferric citrate tablets comprise a combination of two or more of the following: magnesium stearate, calcium stearate, sodium stearyl fumarate. Other suitable lubricants that can be used in the ferric citrate tablets or other oral formulations include one or more of polyethylene glycol (molecular weight above 3350), sodium lauryl sulfate, talc, mineral oil, leucine, and poloxamer. In a specific embodiment, the lubricant used in the ferric citrate tablets or other oral formulations is calcium stearate.

In various aspects, the tablets or other oral formulations can include a disintegrant. The disintegrant can be the same as or different from the binder. By way of example and not limitation, microcrystalline cellulose has both binder and disintegrant properties and microcrystalline cellulose can be used as the sole binder/disintegrant in the tablets and/or oral iron supplements. Examples of other suitable disintegrants include croscarmellose sodium, crospovidone, sodium starch glycolate, and starch.

The binder can be present in the tablets or other oral formulations in an amount ranging from approximately 4.5% by weight to approximately 30% by weight. In certain embodiments, the binder is present in the tablets or other oral formulations in an amount ranging from approximately 5% by weight to approximately 15% by weight. In some embodiments, the binder is present in the tablets or other oral formulations in an amount ranging from approximately 1.0% by weight to approximately 15% by weight. The disintegrant can be present in the tablets or other oral formulations in an amount ranging from approximately 1.5% to by weight to approximately 15% by weight. In various embodiments, some non-starch disintegrants are often used at lower weight percents, e.g., as low as 0.25% and thus the disintegrant present in the tablets or other oral formulations can be as low as 0.25% in some conditions.

The lubricant can be present in the tablets or other oral formulations in an amount ranging from approximately 0.5% by weight to approximately 3% by weight. In certain embodiments, the lubricant is present in the tablets or other oral formulations in an amount ranging from approximately 0.5% by weight to 2% by weight. In some embodiments, the lubricant is present in the tablets or other oral formulations in an amount ranging from approximately 0.5% by weight to approximately 1% by weight. It should be understood that some components, such as microcrystalline cellulose, can function with both disintegrant and binder properties.

The weight of individual tablets or other oral formulations can depend upon the final dosage to be produced; e.g., 125 mg, 250 mg, 500 mg, 667 mg, 750 mg and 1,000 mg of ferric citrate. In some embodiments, the tablets comprise 1 gram of ferric citrate and therefore a dose of 210 mg of ferric iron.

In various embodiments, the ferric citrate tablets or other oral formulations are coated to a weight gain of approximately 2% to 5%. In a specific embodiment, the ferric citrate tablets are coated using an Opadry suspension or equivalent in a perforated pan coater.

In a specific aspect, the tablets and/or oral iron supplements have reduced water content. In one embodiment, the water content of the tablet, as measured by loss on drying (LOD) percentage, is less than 20%. In another embodiment, the water content of the tablet, as measured by LOD %, is less than 19%. In another embodiment, the water content of the tablet, as measured by LOD %, is less than 18%. In another embodiment, the water content of the tablet, as measured by LOD %, is less than 17%. In another embodiment, the water content of the tablet, as measured by LOD %, is less than 16%. In another embodiment, the water content of the tablet, as measured by LOD %, is less than 15%. In another embodiment, the water content of the tablet, as measured by LOD %, is less than 14%. In another embodiment, the water content of the tablet, as measured by LOD %, is less than 13%. In another embodiment, the water content of the tablet, as measured by LOD % is less than 12%. In another embodiment, the water content as measured by LOD % is less than 11%. In another embodiment, the water content as measured by LOD % is less than 10%. In another embodiment, the water content of the tablet, as measured by LOD %, is less than 9%. In another embodiment, the water content of the tablet, as measured by LOD %, is less than 8%. In another embodiment, the water content of the tablet, as measured by LOD %, is less than 7%. In another embodiment, the water content of the tablet, as measured by LOD %, is less than 6%. In another embodiment, the water content of the tablet, as measured by LOD %, is less than 5%.

In certain embodiments, the water content of the tablet, as measured by LOD %, is between 10% and 15%. In some embodiments, the water content of the tablet, as measured by LOD %, is between 5% and 10%. In certain embodiments, the water content of the tablet, as measured by LOD %, is between 5% and 14% in some embodiments, the water content of the tablet, as measured by LOD %, is between 5% and 12%. In certain embodiments, the water content of the tablet, as measured by LOD %, is between 10% and 14%. In some embodiments, the water content of the tablet, as measured by LOD %, is between 2% and 14%. In certain embodiments, the water content of the tablet, as measured by LOD %, is between 2% and 10%. In some embodiments, the water content of the tablet, as measured by LOD %, is between 2% and 12%. In certain embodiments, the water content of the tablet, as measured by LOD %, is between 8% and 10%. In certain embodiments, the water content of the tablet, as measured by LOD %, is between 6% and 9%. In certain embodiments, the water content of the tablet, as measured by LOD %, is between 7% and 9%.

LOD (loss on drying) is a method of thermogravimetric moisture determination. In thermogravimetric processes, the moisture of a material includes substances that volatilize during warming, and therefore contribute to the material's loss of mass. Alongside water this may also include alcohol or decomposition products. When using thermogravimetric measurement methods (drying using infrared, halogen, microwaves or ovens) no distinction is made between water and other volatile components. Technologies known to one of skill in the art can be used to measure LOD. In a specific embodiment, LOD % of the tablet is measured by Mettler-Toledo's model HB-43-S Moisture Balance using the “Standard” drying program, with temperature set at 105° C., endpoint set at mean weight loss of less than 1 mg in 50 seconds, and using samples of 0.9-1.1 gram.

In some embodiments, the tablets or other oral formulations comprise an amount of ferric citrate selected from approximately 1000 mg, approximately 667 mg, approximately 500 mg, approximately 250 mg and approximately 125 mg. In a specific embodiment, the tablets or other oral formulations comprise 1 gram (1000 mg) of ferric citrate. In specific embodiments, the tablets or oral formulations comprise 1 gram of ferric citrate containing approximately 210 mg of ferric iron.

In certain embodiments, the tablets or other oral formulations comprise 1.1 grams of ferric citrate. In some embodiments, the tablets or other oral formulations comprise 1.2 grams of ferric citrate. In certain embodiments, the tablets or other oral formulations comprise 1.3 grams of ferric citrate. In some embodiments, the tablets or other oral formulations comprise 1.5 grams of ferric citrate. In certain embodiments, the tablets or other oral formulations comprise 1.6 grams of ferric citrate. In some embodiments, the tablets or other oral formulations comprise an amount of ferric citrate selected from 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1025 mg, 1050 mg, 1075 mg, 1100 mg, 1125 mg, 1150 mg, 1175 mg, 1200 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg and 2000 mg. In specific embodiments, the tablets or other oral formulations comprise approximately 1 g of ferric citrate. In certain embodiments, the tablets or other oral formulations comprise approximately 1000 mg to 1050 mg, 975 mg to 1050 mg, or 950 mg to 1050 mg of ferric citrate.

In some embodiments, the tablets or other oral formulations comprise between approximately 65 wt % and 92 wt % ferric citrate; between approximately 4.5 wt % and 30 wt % binder; and between 0.5 wt % and 3 wt % lubricant. In certain embodiments, the tablets or other oral formulations comprise between approximately 80 wt % and approximately 92 wt % ferric citrate; between approximately 5 wt % and approximately 15 wt % binder; and between approximately 0.5 wt % and approximately 2 wt % lubricant. In some embodiments, the tablets or other oral formulations comprise between approximately 85 wt % and approximately 92 wt % ferric citrate; between approximately 5 wt % and approximately 15 wt % binder; and between approximately 0.5 wt % and approximately 1 wt % lubricant. In certain embodiments, the lubricant is selected from one or more of magnesium stearate, calcium stearate, and sodium stearyl fumarate. In a specific embodiment, the lubricant is calcium stearate. In specific embodiments, the binder is pregelatinized starch and the lubricant is calcium stearate.

In some embodiments, the tablets or other oral formulations comprise 65% by weight to 92% by weight of ferric citrate and 4.5% by weight to 30% by weight of a binder, wherein the mean surface area to mass ratio of said tablet is equal to or greater than 1 m² per gram, and wherein the LOD % water of the tablet is less than 20% water w/w. In certain embodiments, the mean surface area to mass ratio of the tablets or other oral formulations is equal to or greater than 5 m² per gram. In some embodiments, the mean surface area to mass ratio of the tablets or other oral formulations is equal to or greater than 10 m² per gram. In certain embodiments, the tablets or other oral formulations comprise 70% to 92% by weight of ferric citrate. In some embodiments, the tablets or other oral formulations comprise 80% to 92% by weight of ferric citrate. In certain embodiments, the tablets or other oral formulations comprise 90% to 93% by weight of ferric citrate. In some embodiments, the LOD % water of the tablets or other oral formulations is less than 15% but greater than 2%, 3%, 4% or 5% of water w/w. In some embodiments, the LOD % water of the tablets or other oral formulations is less than 10% but greater than 2%, 3%, 4%, or 5% of water w/w. In some embodiments, the tablets or other oral formulations further comprise a lubricant selected from one or more of magnesium stearate, calcium stearate, and sodium stearyl fumarate. In some embodiments, the tablets or other oral formulations comprise between 0.5% and 3% lubricant. In specific embodiments, the binder comprises pregelatinized starch and the lubricant is calcium stearate. In some embodiments, at least 80% of the ferric citrate in the tablets or other oral formulations is dissolved in a time less than or equal to 60 minutes as measured by test method USP <711>. In certain embodiments, at least 80% of the ferric citrate in the tablets or other oral formulations is dissolved in a time less than or equal to 45 minutes as measured by test method USP <711>. In some embodiments, the tablets or oral formulations comprise approximately 1000 mg of ferric citrate.

In certain embodiments, the tablets or other oral formulations comprise between approximately 80 wt % and approximately 92 wt % ferric citrate and between approximately 5 wt % and approximately 15 wt % binder, wherein the mean surface area to mass ratio of said tablet is equal to or greater than 1 m² per gram, and wherein the LOD % water of the tablet is between 5% to 14%. In some embodiments, the tablets or other oral formulations comprise between approximately 85 wt % and approximately 92 wt % ferric citrate and between approximately 5 wt % and approximately 15 wt % binder, wherein the mean surface area to mass ratio of said tablet is equal to or greater than 1 m^{z} per gram, and wherein the LOD % water of the tablet is between 5% to 14%. In some embodiments, the mean surface area to mass ratio of the tablets or other oral formulations can be equal to or greater than 5 m² per gram. In some embodiments, the mean surface area to mass ratio of the tablets a or other oral formulations is equal to or greater than 10 m² per gram. In some embodiments, the tablets or other oral formulations comprise between approximately 0.5% and approximately 3% lubricant. In certain embodiments, the tablets or other oral formulations comprise between approximately 0.5% and approximately 2% lubricant. In specific embodiments, the binder comprises pregelatinized starch. In another specific embodiment, the lubricant comprises calcium stearate. In some embodiments, at least 80% of the ferric citrate in the tablets or other oral formulations is dissolved in a time less than or equal to 60 minutes as measured by test method USP <711>. In certain embodiments, at least 80% of the ferric citrate in the tablets or other oral formulations is dissolved in a time less than or equal to 45 minutes as measured by test method USP <711>. In some embodiments, the tablets or other oral formulations comprise approximately 1000 mg of ferric citrate. In a specific embodiment, the tablet or other oral formulation comprises a coating.

In certain embodiments, the tablets or other oral formulations comprise between approximately 80 wt % and approximately 92 wt % ferric citrate; between approximately 5 wt % and approximately 15 wt % binder; and between approximately 0.5 wt % and approximately 2 wt % lubricant, wherein at least 80% of the ferric citrate in the tablets or other oral formulations is dissolved in a time less than or equal to 45 minutes, or less than or equal to 60 minutes as measured by test method USP <711>. In some embodiments, the tablets or other oral formulations comprise between approximately 85 wt % and approximately 92 wt % ferric citrate; between approximately 5 wt % and approximately 15 wt % binder, and between approximately 0.5 wt % and approximately 1 wt % lubricant, wherein at least 80% of the ferric citrate in the tablets or other oral formulations is dissolved in a time less than or equal to 45 minutes, or less than or equal to 60 minutes as measured by test method USP <711>. In a specific embodiment, the binder is pregelantinized starch and the lubricant is calcium stearate. In another specific embodiment, the tablet or other oral formulation comprises a coating.

In certain embodiments, the tablets or other oral formulations comprise between approximately 80 wt % and approximately 92 wt % ferric citrate and between approximately 5 wt % and approximately 15 wt % binder, wherein the mean surface area to mass ratio of said tablet is equal to or greater than 1 m² per gram, and wherein the LOD % water of the tablet is between 5% to 10%. In some embodiments, the tablets or other oral formulations comprise between approximately 85 wt % and approximately 92 wt % ferric citrate and between approximately 5 wt % and approximately 15 wt % binder, wherein the mean surface area to mass ratio of said tablet is equal to or greater than 1 m¹ per gram, and wherein the LOD % water of the tablet is between 5% to 10%. In some embodiments, the mean surface area to mass ratio of the tablets or other oral formulations can be equal to or greater than 5 m² per gram. In some embodiments, the mean surface area to mass ratio of the tablets a or other oral formulations is equal to or greater than 10 m² per gram. In some embodiments, the tablets or other oral formulations comprise between approximately 0.5% and approximately 3% lubricant. In certain embodiments, the tablets or other oral formulations comprise between approximately 0.5% and approximately 2% lubricant. In specific embodiments, the binder comprises pregelatinized starch. In another specific embodiment, the lubricant comprises calcium stearate. In some embodiments, at least 80% of the ferric citrate in the tablets or other oral formulations is dissolved in a time less than or equal to 60 minutes as measured by test method USP <711>. In certain embodiments, at least 80% of the ferric citrate in the tablets or other oral formulations is dissolved in a time less than or equal to 45 minutes as measured by test method USP <711>. In some embodiments, the tablets or other oral formulations comprise approximately 1000 mg of ferric citrate. In a specific embodiment, the tablet or other oral formulation comprises a coating.

Table 1 provides a formulation for a ferric citrate tablet according to one embodiment of the present disclosure:

TABLE 1

| | Theoretical | |
|---|---|---|
| Material Description | kg/Batch | % w/w |
| Ferric Citrate | 14.89 | 87.6 |
| Pregelatinized Starch | 1.70 | 10.0 |
| Calcium Stearate | 0.406 | 2.4 |
| Purified Water | 15.30* | N/A* |
| Core Tablet Total | 17.00 | 100.0 |
| Opadry Purple 03K100000 | 0.51 | 15.0 |
| Purified Water | 2.89* | 85.0* |
| Coated Tablet Total | 17.5 | 100.0 |

* - Purified water is removed during a drying phase in the manufacturing process

Table 2 provides a formulation for a ferric citrate tablet according to one embodiment of the present disclosure:

TABLE 2

| | | | | % w/w |
|---|---|---|---|---|
| Material | Target | Theoretical | % w/w | Coated |
| Description | kg/Batch | 100 kg/Lot | Individual | Tablet |
| Ferric Citrate | 14.9 | 80.0-90.0 | 80.0-90.0 | 76.2-88.2 |
| Pregelatinized | 1.7 | 8.0-15.0 | 8.0-15.0 | 7.6-14.7 |
| Starch | | | | |
| Calcium | 0.4 | 1.0-3.0 | 1.0-3.0 | 0.9-2.9 |
| Stearate (1) | | | | |
| OR - Sodium | 0.4 | 2.0-3.0 | 2.0-3.0 | 1.9-2.9 |
| Stearyl | | | | |
| Fumarate (1) | | | | |
| Purified Water | 15.3* | 72.0-135.0* | * | * |
| Core Tablet Total | 17.0 | 100.0 | 100.0 | N/A* |
| Opadry Purple | 0.9 | 5.3 | 15.0 | 2.0-5.0 |
| Purified Water | 5.1* | 30.0* | 85.0* | N/A* |
| Coated Tablet | 17.5 to 17.9 | 35.3 | 100.0 | 100.0 |
| Total | | | | |

(1) - use either calcium stearate or sodium stearyl fumarate as lubricant
* - Purified water is removed

Table 3 provides a formulation for a ferric citrate tablet according to one embodiment of the present disclosure:

TABLE 3

| Material Description | Target kg/Batch | % w/w Individual |
|---|---|---|
| Ferric Citrate | 14.89 | 87.6 |
| Pregelatinized Starch | 1.70 | 10.0 |
| Calcium Stearate (1) | 0.406 | 2.4 |
| Purified Water | 15.30 | N/A |
| Core Tablet Total | 17.00 | 100.0 |
| Opadry Purple | 0.51 | 15.0 |
| Purified Water | 2.89 | 85.0 |
| Coated Tablet Total | 17.5 | 100.0 |

Table 4 provides a formulation for a ferric citrate oral formulation according to one embodiment of the present disclosure:

TABLE 4

| Material/Component | Formula Composition % w/w |
|---|---|
| Ferric Citrate | 70.0 to 99.0 |
| Starch | 0.0 to 30.0 |
| Microcrystalline Cellulose | 0.0 to 30.0 |
| Polyvinylpyrrolidone | 0.0 to 30.0 |
| Calcium Stearate | 0.0 to 3.0 |
| Sodium Stearyl Fumarate | 0.0 to 3.0 |
| Purified Water | N/A* |
| Core Caplet Total | 100.0 |
| Film coating | 0.0 to 5.0 |
| Purified Water | N/A* |
| Coated Caplet Total | 100.0 |

*The purified water is removed.

Table 5 provides a formulation for a ferric citrate oral formulation according to one embodiment of the present disclosure:

TABLE 5

| Material | Weight mg ± 10% |
|---|---|
| Ferric Citrate | 1,500 |
| Starch | 150 |
| Microcrystalline Celluose | 0 |
| Polyvinylpyrrolidone | 0 |
| Calcium Stearate | 16 |
| Sodium Stearyl Fumarate | 0 |
| Purified Water | N/A* |
| Core Caplet Total - mg | 1,666 |
| Film coating | 50 |
| Purified Water | N/A* |
| Coated Caplet Total - mg | 1,766 |

*The purified water is removed.

In a specific embodiment, the ferric citrate tablet is the ferric citrate tablet referred to as JTT-751 (Japan Tobacco Inc. and Torii Pharmaceutical Co., Ltd.). In another specific embodiment, the ferric citrate tablet is the Auryxia™ tablet sold by Keryx Biopharmaceuticals, Inc.

### 4.6. Methods of Assessing Iron Storage Parameters

As stated above, iron storage parameters may be measured to determine whether an IDA patient has sufficient iron stores to maintain adequate health. These iron storage parameters are useful in assessing whether an IDA patient can be suitably treated with ferric citrate and the efficacy of ferric citrate treatment so as to guide health care professionals in determining and/or adjusting a dosage regimen for the patient. To assess the one or more iron storage parameters, a blood sample may be drawn by needle from a vein in the arm and iron tests (i.e., iron studies) as well as complete blood count tests may be performed to determine the amount of circulating iron in the blood, the capacity of the blood to transport iron, and the amount of stored iron in tissues. In some embodiments, the one or more iron storage parameters are selected from hematocrit, hemoglobin (Hb) concentration, total iron-binding capacity (TIBC), TSAT, serum iron levels, liver iron levels, spleen iron levels, and serum ferritin levels. In specific embodiments, the one or more iron storage parameters are hemoglobin concentration, TSAT, or serum ferritin levels.

### 5. EXAMPLES

The following examples in this Section (i.e., Section 5) describe the use of ferric citrate to treat IDA. In particular, Example 1 demonstrates the use of ferric citrate to achieve a clinically significant increase in hemoglobin concentration in IDA patients in the absence of erythropoiesis-stimulating agents and intravenous iron Surprising, a low dose of ferric citrate taken without food was well tolerated and resulted in a clinically significant increase in hemoglobin concentration in IDA patients.

The examples are offered by way of illustration, and not by way of limitation.

### 5.1. Example 1: A Phase 2 Pilot Study of KRX-0502 (Ferric Citrate Coordination Complex) in Treating IDA in Patients with Stage 3-5 Non-Dialysis Dependent Chronic Kidney Disease (NDD-CKD)

### 5.1.1. Protocol

The objective of the study was to evaluate the efficacy and safety of Auryxia™ (Ferric Citrate; Keryx Biopharmaceuticals, Inc.) in treating IDA in subjects with stage 3-5 non-dialysis dependent chronic kidney disease (NDI)-CKD) as measured by changes in hemoglobin over an 8-week treatment period. The primary endpoint for the study was change in hemoglobin concentration from baseline (Day 0) to end of the 8-week treatment period (Week 8). Secondary endpoints for the study included the mean change from baseline to the highest hemoglobin value; percentage of subjects achieving hemoglobin change ≥1.0 g/dl at any visit during the study; and the percentage of patients achieving hemoglobin ≥12.0 g/dl hemoglobin at any visit during the study.

### 5.1.1.1. Overall Design

This was a Phase 2, single-arm, multicenter, open-label clinical trial.

Following a screening visit, eligible subjects were enrolled and received a fixed starting dose of Auryxia™ (Ferric Citrate; Keryx Biopharmaceuticals, Inc.) of 1 tablet/day without food. All subjects had to have a hemoglobin ≥9.0 g/dl and ≤11.5 g/dl at their screening visit to enter the 8-week treatment period.

After starting treatment at Day 0 with Auryxia™ (Ferric Citrate; Keryx Biopharmaceuticals, Inc.) at the starting dose of 1 tablet/day, hemoglobin was measured at every study visit. Subjects who had a hemoglobin increase <1.0 g/dl after the first 4 weeks compared to baseline (Day 0), were titrated up to 2 tablets/day for the remainder of the trial. Subjects who had a hemoglobin increase >1.5 g/dl after the first 4 weeks, compared to baseline (Day 0) were to be titrated down to a dose of 1 tablet every other day for the remainder of the trial (one subject had a hemoglobin increase >1.5 g/dl after the first 4 weeks compared to baseline (Day 0), however the subject remained on a dose of 1 tablet/day for the remainder of the trial, due to a request by the Principle Investigator (PI) to deviate from the protocol). Otherwise, subjects remained on a dose of 1 tablet/day for the remainder of the trial (two subjects had a hemoglobin increase >1.0 g/dl and <0.15 g/dl after the first 4 weeks compared to baseline (Day 0); one of the two subjects remained on a dose of 1 table/day for the remainder of the trial, the other subject was titrated up to 2 tablets/day for the remainder of the trial).

The use of phosphate binders was not permitted at any time during the trial. The use of oral or 1V iron and Erythropoiesis-Stimulating Agents (ESAs) and receipt of blood transfusions was not permitted at any time during the trial.

Blood samples for Complete Chemistry Profile (CCP), iron studies, and Complete Blood Count (CBC) were collected at screening; at Day 0; and at 1, 2, 4, 6 and 8 weeks after treatment began.

### 5.1.1.2. Patient Population/Inclusion and Exclusion Criteria

Human subjects were screened and 32 human subjects were enrolled in the study. Eligible subjects received a starting fixed dose of 1 tablet/day of Auryxia™ (Ferric Citrate; Keryx Biopharmaceuticals, Inc.) without food. Subjects who had a hemoglobin increase <1.0 g/dl after the first 4 weeks compared to baseline (Day 0), were titrated up to 2 tablets/day for the remainder of the trial. One subject who had a hemoglobin increase ≥1.0 g/dl and ≤1.5 g/dl after the first 4 weeks compared to baseline (Day 0) was also titrated up to 2 tablets/day for the remainder of the trial. The other subject who had a hemoglobin increase ≥1.0 g/dl and ≤1.5 g/dl after the first 4 weeks compared to baseline (Day 0) remained on a dose of 1 tablet/day for the remainder of the trial. One subject had a hemoglobin increase >1.5 g/dl after the first 4 weeks compared to baseline (Day 0), and remained on a dose of 1 tablet/day for the remainder of the trial, due to a request by the PI to deviate from the protocol.

Following a screening visit, eligible subjects enrolled in an 8-week treatment period. Enrollment into the study (Day 0) generally occurred within a week of the screening visit.

Inclusion Criteria

Subjects enrolled in the study met the following inclusion criteria:
1. Males and non-lactating females with negative scrum pregnancy test (for females of child-bearing potential) at screening visit
2. Age >18 years
3. Serum ferritin <300 ng/ml and TSAT <25% at screening visit
4. Hemoglobin ≥9.0 g/dl and ≤11.5 g/dl at screening visit
5. eGFR <60 ml/min at screening visit using the 4-variable Modification of Diet in Renal Disease (MDRD) equation

Exclusion Criteria

Subjects who meet any of the following exclusion criteria were not enrolled into this study:
1. Subjects receiving phosphate binder medication(s) at, or within 4 weeks prior to, screening
2. Symptomatic gastrointestinal bleeding, inflammatory bowel disease, inflammatory bowel syndrome and/or Crohn's Disease within 24 weeks prior to screening visit
3. Evidence of acute kidney injury or requirement for dialysis within 8 weeks prior to screening visit
4. Kidney transplant anticipated or start of dialysis expected within 16 weeks of screening visit
5. Intravenous iron administered within 4 weeks prior to screening visit
6. Erythropoiesis-Stimulating Agent (ESA) administered within 4 weeks prior to screening visit
7. Blood transfusion within 4 weeks prior to screening visit
8. Receipt of any investigational drug within 4 weeks prior to screening visit
9. Cause of anemia other than iron deficiency or chronic kidney disease
10. History of malignancy in the last five years (treated cervical or skin cancer may be permitted if approved by Keryx)
11. History of hemochromatosis
12. Active drug or alcohol dependence or abuse (excluding tobacco use) within the 12 months prior to screening visit or evidence of such abuse
13. Subjects with any known allergies to iron products
14. Previous intolerance to oral ferric citrate
15. Psychiatric disorder that interferes with the subject's ability to comply with the study protocol
16. Planned surgery or hospitalization during the trial
17. Any other medical condition that, in the opinion of the PI, renders the subject unable to or unlikely to complete the trial or that would interfere with optimal participation in the trial or produce significant risk to the subject
18. Inability to cooperate with study personnel or history of noncompliance

### 5.1.1.3. Drug Administration and Titration

The active ingredient in Auryxia™ (Ferric Citrate; Keryx Biopharmaceuticals, Inc.) tablet is chemically known as iron (+3), x (1, 2, 3-propanetricarboxylic acid, 2-hydroxy-), y (H₂O)

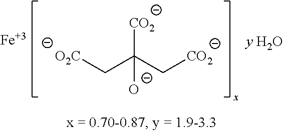

The Auryxia™ (Ferric Citrate; Keryx Biopharmaceuticals, Inc.) is a tablet contains 210 mg of ferric iron, equivalent to 1 gram of ferric citrate.

Subjects who had a hemoglobin increase of <1.0 g/dl after the first 4 weeks compared to Day 0 were titrated up to 2 tablets/day for the remainder of the study. One subject who had a hemoglobin increase ≥1.0 g/dl and ≤1.5 g/dl after the first 4 weeks compared to Day 0 was also titrated up to 2 tablets/day for the remainder of the trial. The other subject who had a hemoglobin increase ≥1.0 g/dl and ≤ 1.5 g/dl after the first 4 weeks compared to Day 0 remained on a dose of 1 tablet/day for the remainder of the trial. One subject had a hemoglobin increase of >1.5 g/dl after the first 4 weeks compared to Day 0, and remained on a dose of 1 tablet/day for the remainder of the trial, due to a request by the Principle Investigator (PI) to deviate from the protocol.

The maximum number of Auryxia™ (Ferric Citrate; Keryx Biopharmaceuticals, Inc.) tablets per day permitted was 2, or 2 g/day. The Principal Investigator (PI) was permitted to reduce the dose of study drug due to an adverse event in consultation with Keryx Biopharmaceuticals, Inc.

Subjects took Auryxia™ (Ferric Citrate; Keryx Biopharmaceuticals, Inc.) orally without meals. Subjects were instructed not to take Auryxia™ (Ferric Citrate; Keryx Biopharmaceuticals, Inc.) if less than two hours had passed since the ingestion of their meals or snacks. Subjects were advised to try to take their daily dose at approximately the same time during each day. Daily water-soluble multivitamins (i.e., Centrum, Nephrocaps, Renaphro, etc.) were allowed during the study. Subjects were advised to take multivitamins separately (at least two hours apart) from Auryxia™ (Ferric Citrate; Keryx Biopharmaceuticals, Inc.). Subjects were encouraged to maintain a stable dose and type of multivitamin (if any) throughout the trial. Subjects were advised to take calcium supplements separately (at least two hours apart) from Auryxia™ (Ferric Citrate; Keryx Biopharmaceuticals, Inc.).

### 5.1.1.4. Study Drug Discontinuation

Subjects were permitted to stop study drug for any of the following reasons:
1. Intercurrent illness, medical event or hospitalization necessitating study drug discontinuation
2. Investigator's discretion for the best interest of the subject

If study drug is discontinued due to an intercurrent illness or adverse event that resolves, the subject may be given the study drug again for the remainder of their trial participation.

### 5.1.1.4.1 Early Termination

Subjects were permitted to discontinue the trial for the following reasons:
1. Subject request
2. Lost to follow-up
3. Sponsor or investigator decision to terminate the trial at any time
4. Start of dialysis
5. Pregnancy
6. Kidney transplantation
7. Meeting the pre-specified early termination criteria (see below)
8. Safety
9. Death
10. Other

If a subject's Hgb is <9.0 or >13.0 g/dl for two consecutive study visits (at least 7 days apart) during the 8-week treatment period after Day 0, the subject was instructed to stop study drug and exit the trial.

If a subject early terminates from the trial for any reason, the subject should be encouraged to complete the final visit assessments.

### 5.1.1.4.2. Adverse Events

All adverse events were to be recorded. An adverse event (AE) was defined to be any reaction, side effect, or other undesirable event that occurs in conjunction with the use of a drug, biologic product or diagnostic agent in humans, whether or not the event is considered drug related. In this trial, this included any illness, sign, symptom or clinically significant laboratory test abnormality that has appeared or worsened during the course of the clinical trial, regardless of causal relationship to the drug(s) under study. Following the questioning and examination of the subject, all AEs were required to be noted. If known, the name of the underlying illness or disorder (i.e., the diagnosis) was requested to be recorded, rather than its individual symptoms.

Subjects experiencing AEs that cause interruption or discontinuation of trial medication, or those experiencing adverse events that are present at the end of their participation in the trial should receive follow-up as appropriate (to resolution or stabilization).

Severity of an AE was defined as a qualitative assessment of the degree of intensity of an AE as determined by the investigator or reported to him/her by the subject. The assessment of severity was made irrespective of drug relationship or seriousness of the event and should be evaluated according to the following scale:
1=Mild (discomfort noticed, but no disruption of normal daily activity.)
2=Moderate (discomfort sufficient to reduce or affect normal daily activity.)
3 Severe (incapacitating, with inability to work or to perform normal daily activity.)

Non-Serious Adverse Events

Any adverse event that was not designated as serious, as defined below, was required to be recorded.

Serious Adverse Events

An event that was serious was required to be recorded and marked as “serious.” An serious adverse event (SAE) was one that met any one of the following criteria:
Results in death
Is a Life-threatening experience,
Requires or prolongs inpatient hospitalization defined as >24-hour hospitalization
Causes persistent or significant disability/incapacity
Results in congenital anomaly
Is an important medical event that may jeopardize the subject and may require medical or surgical intervention to prevent one of the outcomes listed above

Life-threatening experience: Any adverse event that places the subject, in the view of the investigator, at immediate risk of death from the adverse event as it occurred (i.e., does not include an adverse event that had it occurred in a more severe form, might have caused death).

Persistent or significant disability/incapacity: Any adverse event that may result in a substantial disruption of a person's ability to conduct normal life functions.

Important medical event: Any adverse event that may jeopardize the subject and may require medical or surgical intervention to prevent one of the outcomes listed above. Adverse events that may not result in death, be life-threatening, or require hospitalization may be considered an SAE when, based upon appropriate medical judgment, they may jeopardize the subject and may require medical or surgical intervention to prevent one of the outcomes listed above.

A subject experiencing 1 or more SAEs was to receive treatment and follow-up evaluations by the investigator or was to be referred to another appropriate physician for treatment and follow-up. SAEs were to be monitored from the time of consent and for up to 28 days after the subject has discontinued study drug.

All adverse events, whether serious or non-serious, were to be followed to resolution (or stabilization, if applicable) or until the adverse event was determined by the investigator to be no longer clinically significant.

### 5.1.1.5. Laboratory Outcomes of Interest

A laboratory outcome of interest was one that met any one of the following criteria:
Ferritin ≥800 ng/ml
TSAT≥50%
Liver enzyme elevations≥3× the upper limit of normal (ULN)

### 5.1.1.6. Analysis Population

Efficacy

26 subjects completed the 8-week treatment period on study drug. The efficacy analyses were based on data from the 26 subjects.

Safety

The safety analyses were based on the safety population that consisted of all subjects who take at least one dose of study drug.

### 5.1.2. Results

Fifty eight subjects were screened and 32 subjects were enrolled. All 32 subjects received at least 1 dose of Auryxia™ (Ferric Citrate; Keryx Biopharmaceuticals, Inc.) and were included in the Safety Population. Twenty six subjects (81.3%) completed the study and were included in the Analysis Population. Six subjects (18.8%) early terminated, 3 subjects (9.4%) due to an adverse event, 1 (3.1%) due the investigator judgment, and 2 (6.3%) due to other reasons. The majority of subjects in this trial were White/Caucasian (96.9%), Male (53.1%), aged 65 years or older and had Stage 3 CKD (43.8%).

Twenty six subjects completed the 8-week treatment period (81.3%) and were included in the analysis population. The mean and median duration of exposure in this trial were 40.2 and 42.0 days, respectively. The mean and median dose of Auryxia™ (Ferric Citrate; Keryx Biopharmaceuticals, Inc.) was 1.2 g per day. Overall, laboratory values for non-iron related parameters were similar to those at baseline throughout the study.

Treatment with Auryxia™ (Ferric Citrate; Keryx Biopharmaceuticals, Inc.) for 8 weeks resulted in a statistically significant increase in hemoglobin, from 10.8±0.7 g/dl at baseline to 11.2±0.9 g/dl at Week 8 (P=0.0212). See Table 6, infra. The mean change in hemoglobin from baseline to the highest value was 0.6 g/dl (P<0.0001) Six subjects (23.1%) had an increase in hemoglobin of at least 1.0 g/dl compared to baseline at any time during the study and 7 subjects (26.9%) achieved a Hemoglobin ≥12.0 g/dl at least once during the study. See Table 7, infra.

In addition, treatment with Auryxia™ (Ferric Citrate; Keryx Biopharmaceuticals, Inc.) for 8 weeks resulted in increases in iron storage parameters, serum ferritin and TSAT values, compared to baseline. Serum ferritin levels increased by an average of 35 ng/ml, from 84.9164.7 ng/ml at baseline to 120.1±82.5 ng/ml at Week 8, p-value 0.001, in subjects taking Auryxia™ (Ferric Citrate; Keryx Biopharmaceuticals, Inc.). See Table 8, infra, TSAT values increased an average of 5.7%, from 19.2±6.5% to 24.9±8.5%, p-value 0.003, in subject taking Auryxia™ (Ferric Citrate; Keryx Biopharmaceuticals, Inc.).

Thus, administration of Auryxia™ (Ferric Citrate; Keryx Biopharmaceuticals, Inc.) without food was generally safe and well tolerated in this study. Treatment with Auryxia™ (Ferric Citrate; Keryx Biopharmaceuticals, Inc.) for 8 weeks resulted in a significant increase in hemoglobin as well as in serum ferritin levels and TSAT values.

TABLE 6
Hemoglobin Concentrations

| | N | Mean (SD) | P-Value |
|---|---|---|---|
| Baseline | 26 | 10.8 (0.7) | — |
| Week 8 | 26 | 11.2 (0.9) | 0.0212 |
| Highest Value | 26 | 11.4 (0.7) | <0.0001 |

TABLE 7
Patients with >=1.0 g/dl Increase in Hemoglobin Concentrations
and Patients with >=12.0 g/dl Hemoglobin Concentrations

| | | KRX-0502 |
|---|---|---|
| Item | Stat | (N = 26) |
| Change >=1.0 g/dl at any visit | n (%) | 6 (23.1) |
| Value >=12.0 g/dl at any visit | n (%) | 7 (26.9) |

TABLE 8
Serum Ferritin Levels

| | | Summary Statistics (N = 26) | | | | | | | | |
|---|---|---|---|---|---|---|---|---|---|---|
| Parameter | Visit | n | Mean | SD | Median | P(25) | P(75) | Min | Max | P-value |
| Ferritin | Baseline | 26 | 84.9 | 64.66 | 72.0 | 31.0 | 121.0 | 8 | 275 | . |
| (ng/ml) | Visit 3 | 26 | 91.6 | 64.58 | 77.5 | 43.0 | 125.0 | 13 | 310 | . |
| | Visit 4 | 26 | 91.7 | 63.68 | 71.5 | 51.0 | 131.0 | 19 | 303 | . |
| | Visit 5 | 26 | 92.2 | 62.02 | 89.0 | 45.0 | 117.0 | 21 | 261 | . |
| | Visit 6 | 26 | 99.7 | 61.99 | 85.5 | 64.0 | 132.0 | 17 | 260 | . |
| | Visit 7 | 26 | 120.1 | 82.53 | 85.5 | 63.0 | 163.0 | 23 | 340 | . |
| Ferritin | Visit 3 | 26 | 6.7 | 22.90 | 2.5 | −3.0 | 12.0 | −41 | 65 | 0.1465 |
| (ng/ml) | Visit 4 | 26 | 6.8 | 25.72 | 11.5 | −10.0 | 20.0 | −60 | 60 | 0.1916 |
| Change from | Visit 5 | 26 | 7.3 | 20.60 | 7.5 | −4.0 | 16.0 | −30 | 54 | 0.0811 |
| Baseline | Visit 6 | 26 | 14.8 | 24.03 | 16.5 | −8.0 | 31.0 | −36 | 55 | 0.0043 |
| | Visit 7 | 26 | 35.2 | 48.38 | 30.5 | 15.0 | 43.0 | −64 | 188 | 0.0010 |

### 5.2. Example 2: Animal Models For Colitis

To evaluate the ability of ferric citrate to treat IDA in subjects with an inflammatory bowel condition, animal models of colitis are administered ferric citrate and the effect of the ferric citrate on iron storage parameters, such as hemoglobin concentration and TSAT values, are determined.

T-cell Transfer Model of Chronic Colitis

Chronic colonic inflammation is induced in mice by the adoptive transfer of IL-102/2 CD4⁺ T-cells into RAG2/2 recipients. Briefly, RAG2/2 recipient mice at an age of 2-3 months are injected with 10⁶ CD4⁺ T-cells obtained from IL-102/2 donor mice, with the T cells enriched (90%; from single-cell suspensions of splenocytes) by negative selection using a commercially available kit. Additional age-matched RAG2/2 mice and C57BL/6 mice are treated identically except for the injection of vehicle alone (phosphate-buffered saline [PBS]) Instead of the T cells. At 8-week post-injection, the mice are used for treatment with ferric citrate or control.

DSS Model of Acute/Self-Limiting Colitis

Acute colonic inflammation is induced in 2-month to 3-month C57BL/6 mice via administration of 5% dextran sulphate sodium (DSS) in drinking water for 6 days. The DSS is added to water that is filter purified. Filtered water (without DSS) is administered for 6 days to age matched C57BL/6 mice as a control group. At the end of the DSS administration, the mice are used for treatment with ferric citrate or control.

Both the T-cell transfer model of colitis and the DSS model of Colitis are known to induce significant decreases in hematocrit, blood hemoglobin, and TSAT, with the spleen and liver showing a decrease in iron content in the T-cell transfer model of colitis. In addition, both models of colitis have demonstrated significant increases in plasma erythropoietin and plasma iron-binding capacities.

Treatment Group

After colitis has been induced, a certain number of mice are administered ferric citrate via oral gavage or dietary administration at doses corresponding to the human effective doses. As a control, a certain number of mice are administered ferrous sulfate via oral gavage or dietary administration. Prior to administration of ferric citrate and a certain number of days (e.g., 1, 2, 3, 4, 5, 6 or more days) or weeks (e.g., 1, 2, 3, 4, 5 or more weeks) after administration of ferric citrate or control, iron and hematology analysis is conducted.

Iron and Hematology Analysis

The mice are anesthetized with an intraperitoneal injection of 150 mg/kg ketamine and 10 mg/kg xylazine. A blood sample is withdrawn from the cannulated right carotid artery with a portion mixed with the anticoagulant EDTA for measures of hematocrit, hemoglobin concentration, and hemoglobin per RBC, and the remaining untreated blood processed for measures of serum iron, unsaturated iron-binding capacity, total iron-binding capacity (TIBC), transferrin saturation, serum ferritin, and plasma erythropoietin (all measures obtained with an Hematology Analyzer). After euthanasia, tissue sections (or entire organs in some cases) are dissected for iron measurements

Finally, it should be noted that there are alternative ways of implementing the embodiments disclosed herein. Accordingly, the present embodiments are to be considered as illustrative and not restrictive. Furthermore, the claims are not to be limited to the details given herein, and are entitled their full scope and equivalents thereof.

All references cited herein are incorporated herein by reference in their entirety and for all purposes to the same extent as if each individual publication or patent or patent application was specifically and individually indicated to be incorporated by reference in its entirety for all purposes.

1. A method for treating iron deficiency anemia in a human patient, wherein the patient has not been diagnosed with chronic kidney disease and/or and the patient has a serum ferritin level of between 5 ng/ml to 300 ng/ml, the method comprising orally administering a ferric citrate tablet containing approximately 210 mg of ferric iron to the patient, wherein the ferric citrate in the tablet is a complex of iron (+3), 0.70-0.87 (1, 2, 3-propanetricarboxylic acid, 2-hydroxy-), 1.9-3 (H₂O).

2.-4. (canceled)

5. A method for treating iron deficiency anemia in a human patient, wherein the patient has not been diagnosed with chronic kidney disease and the patient has a serum ferritin level of between 5 ng/ml to 300 ng/ml, the method comprising orally administering a ferric citrate tablet containing approximately 210 mg of ferric iron to the patient, wherein the ferric citrate is not administered within 2 hours of food being ingested by the patient, and wherein the ferric citrate in the tablet is a complex of iron (+3), 0.70-0.87 (1, 2, 3-propanetricarboxylic acid, 2-hydroxy-), 1.9-3 (H₂O).

6. The method of claim 1, wherein the patient has a serum ferritin level of between 5 ng/ml to 250 ng/ml, 5 ng/ml to 150 ng/ml, or 5 ng/ml to 100 ng/ml.

7.-8. (canceled)

9. The method of claim 1, wherein the patient has a serum ferritin level of between 5 ng/ml to 75 ng/ml or 5 ng/ml to 50 ng/ml.

10. (canceled)

11. The method of claim 1, wherein the patient has a serum ferritin level of between 5 ng/ml to 25 ng/ml.

12. The method of claim 1, wherein the patient has a serum ferritin level of between 5 ng/ml to 15 ng/ml.

13. The method of claim 1, wherein the patient has a serum ferritin level of between 5 ng/ml to 10 ng/ml.

14. A method for treating iron deficiency anemia in a human patient that has not been diagnosed with chronic kidney disease, the method comprising:
(a) orally administering to the patient one ferric citrate tablet containing approximately 210 mg of ferric iron per day, wherein the ferric citrate is not administered within 2 hours of food being ingested by the patient, and wherein the ferric citrate in the tablet is a complex of iron (+3), 0.70-0.87 (1, 2, 3-propanetricarboxylic acid, 2-hydroxy-), 1.9-3 (H₂O); and
(b) decreasing the dose of ferric citrate after 4 weeks if the hemoglobin concentration of the subject has increased by more than 5 g/dl and increasing the dose of ferric citrate after 4 weeks if the hemoglobin concentration of the subject has increased by less than 1 g/dl.

15. The method of claim 1, wherein the patient has a gastrointestinal disorder.

16. The method of claim 15, wherein the gastrointestinal disorder is inflammatory bowel disease, inflammatory bowel syndrome, Crohn's disease, ulcerative colitis, microscopic colitis, or chemically-induced colitis.

17. The method of claim 16, wherein the microscopic colitis is collagenous colitis or lymphocytic colitis.

18. The method of claim 16, wherein the chemically-induced colitis is NSAID (nonsteroidal anti-inflammatory drug)-induced colitis.

19. The method of claim 1, wherein the patient has blood loss.

20. The method of claim 19, wherein the blood loss is associated with childbirth or menstruation.

21. The method of claim 19, wherein the blood loss is associated with an infection.

22. The method of claim 1, wherein the patient has insufficient dietary intake of iron.

23. The method of claim 1, wherein the patient has insufficient absorption of iron.

24. The method of claim 1, wherein the patient is monitored for one or more iron storage parameters.

25. The method of claim 24, wherein the one or more iron storage parameters is selected from the group consisting of hemoglobin concentration, serum ferritin level, TSAT value, serum iron level, hematocrit level, TIBC value, plasma erythropoietin level, and FEP level.
